SEQUENCE LISTING (159 sequences)

SEQ ID NO: 1 - skipped

SEQ ID NO: 2 - protein, 13 aa, synthetic construct
  SITE 2: note = X is W or A
  SITE 5: note = X is D or S
  SITE 6: note = X is S or D
  SITE 10: note = X is X5 which is any amino acid residue, including modified and non-natural amino acids
CXPLXXYWCXRIC

SEQ ID NO: 3 - protein, 15 aa, synthetic construct
  SITE 9: note = X is F or W
  SITE 10: note = X is L or V
  SITE 11: note = X is H, R or T
  SITE 13: note = X is L or G
  SITE 14: note = X is G, Q or H
CVTTSYDCXXXLXXC

SEQ ID NO: 4 - skipped

SEQ ID NO: 5 - protein, 13 aa, synthetic construct
  SITE 2: note = X is W, A or Y
  SITE 3: note = X is P or A
  SITE 5: note = X is D, S or A
  SITE 6: note = X is S, D, P or A
  SITE 8: note = X is W or 1-Nal
  SITE 10: note = X is X5 which is any amino acid residue, including modified and non-natural amino acids
  SITE 11: note = X is R, HArg or A
CXXLXXYXCXXIC

SEQ ID NO: 6 - protein, 15 aa, synthetic construct
CPFGCMETWSWPIWC

SEQ ID NO: 7 - protein, 15 aa, synthetic construct
CPFGCMRGWSWPIWC

SEQ ID NO: 8 - protein, 15 aa, synthetic construct
CPFGCMSGWSWPIWC

SEQ ID NO: 9 - protein, 15 aa, synthetic construct
CPFGCMEGWSWPIWC

SEQ ID NO: 10 - protein, 15 aa, synthetic construct
CPFGCMEDWSWPIWC

SEQ ID NO: 11 - protein, 15 aa, synthetic construct
CPFGCMPGWSWPIWC

SEQ ID NO: 12 - protein, 15 aa, synthetic construct
CPFGCMKSWSWPIWC

SEQ ID NO: 13 - protein, 15 aa, synthetic construct
CPFGCMKTWSWPIWC

SEQ ID NO: 14 - protein, 15 aa, synthetic construct
CPFGCMKGWSWPIWC

SEQ ID NO: 15 - protein, 15 aa, synthetic construct
CPFGCQEHWSWPIWC

SEQ ID NO: 16 - protein, 15 aa, synthetic construct
CPFGCIKSWSWPIWC

SEQ ID NO: 17 - protein, 15 aa, synthetic construct
CPFGCQEDWSWPIWC

SEQ ID NO: 18 - protein, 15 aa, synthetic construct
CPFGCMSDWSWPIWC

SEQ ID NO: 19 - protein, 15 aa, synthetic construct
  SITE 7: note = X is HArg
CPFGCMXNWSWPIWC

SEQ ID NO: 20 - protein, 15 aa, synthetic construct
  SITE 7: note = X is K(Ac)
CPFGCMXNWSWPIWC

SEQ ID NO: 21 - protein, 15 aa, synthetic construct
  SITE 7: note = X is K(Ac)
CPFGCMXSWSWPIWC

SEQ ID NO: 22 - protein, 15 aa, synthetic construct
  SITE 6: note = X is Nle
CPFGCXKSWSWPIWC

SEQ ID NO: 23 - protein, 15 aa, synthetic construct
  SITE 7: note = X is HArg
CPFGCMXSWSWPIWC

SEQ ID NO: 24 - protein, 15 aa, synthetic construct
CPFGCMKSWSWPIWC

SEQ ID NO: 25 - protein, 15 aa, synthetic construct
CPAGCMKNWSWPIWC

SEQ ID NO: 26 - protein, 15 aa, synthetic construct
CPFACMKNWSWPIWC

SEQ ID NO: 27 - protein, 15 aa, synthetic construct
CPFGCMANWSWPIWC

SEQ ID NO: 28 - protein, 15 aa, synthetic construct
CPFGCMKAWSWPIWC

SEQ ID NO: 29 - protein, 15 aa, synthetic construct
CPFGCMKNASWPIWC

SEQ ID NO: 30 - protein, 15 aa, synthetic construct
CPFGCMKNWSWPAWC

SEQ ID NO: 31 - protein, 15 aa, synthetic construct
CAFGCMKNWSWPIWC

SEQ ID NO: 32 - protein, 15 aa, synthetic construct
  SITE 6: note = X is tBuAla
CPFGCXKNWSWPIWC

SEQ ID NO: 33 - protein, 15 aa, synthetic construct
  SITE 6: note = X is HLeu
CPFGCXKNWSWPIWC

SEQ ID NO: 34 - protein, 15 aa, synthetic construct
  SITE 14: note = X is 1Nal
CPFGCMKNWSWPIXC

SEQ ID NO: 35 - protein, 15 aa, synthetic construct
  SITE 7: note = X is HArg
CPFDCMXNWSWPIWC

SEQ ID NO: 36 - protein, 15 aa, synthetic construct
  SITE 7: note = X is HArg
CPFACMXNWSWPIWC

SEQ ID NO: 37 - protein, 15 aa, synthetic construct
  SITE 3: note = X is 3MePhe
CPXGCMKNWSWPIWC

SEQ ID NO: 38 - protein, 15 aa, synthetic construct
  SITE 3: note = X is 4MePhe
CPXGCMKNWSWPIWC

SEQ ID NO: 39 - protein, 15 aa, synthetic construct
  SITE 3: note = X is HPhe
CPXGCMKNWSWPIWC

SEQ ID NO: 40 - protein, 15 aa, synthetic construct
CPFDCMKNWSWPIWC

SEQ ID NO: 41 - protein, 15 aa, synthetic construct
  SITE 6: note = X is Hse(Me)
CPFGCXKNWSWPIWC

SEQ ID NO: 42 - protein, 15 aa, synthetic construct
  SITE 9: note = X is AzaTrp
CPFGCMKNXSWPIWC

SEQ ID NO: 43 - protein, 15 aa, synthetic construct
CPFGCMKNWSFPIWC

SEQ ID NO: 44 - protein, 15 aa, synthetic construct
CPFGCMKNWSYPIWC

SEQ ID NO: 45 - protein, 15 aa, synthetic construct
  SITE 11: note = X is 1Nal
CPFGCMKNWSXPIWC

SEQ ID NO: 46 - protein, 15 aa, synthetic construct
  SITE 11: note = X is 2Nal
CPFGCMKNWSXPIWC

SEQ ID NO: 47 - protein, 15 aa, synthetic construct
  SITE 11: note = X is AzaTrp
CPFGCMKNWSXPIWC

SEQ ID NO: 48 - protein, 15 aa, synthetic construct
  SITE 12: note = X is Aze
CPFGCMKNWSWXIWC

SEQ ID NO: 49 - protein, 15 aa, synthetic construct
  SITE 12: note = X is Pip
CPFGCMKNWSWXIWC

SEQ ID NO: 50 - protein, 15 aa, synthetic construct
CPFGCMKNWSWPIFC

SEQ ID NO: 51 - protein, 15 aa, synthetic construct
CPFGCMKNWSWPIYC

SEQ ID NO: 52 - protein, 15 aa, synthetic construct
  SITE 14: note = X is AzaTrp
CPFGCMKNWSWPIXC

SEQ ID NO: 53 - protein, 15 aa, synthetic construct
CGFGCMKNWSWPIWC

SEQ ID NO: 54 - protein, 15 aa, synthetic construct
  SITE 2: note = X is Aze
CXFGCMKNWSWPIWC

SEQ ID NO: 55 - protein, 15 aa, synthetic construct
  SITE 4: note = X is K(Ac)
CPFXCMKNWSWPIWC

SEQ ID NO: 56 - protein, 15 aa, synthetic construct
CPFGCLKNWSWPIWC

SEQ ID NO: 57 - protein, 15 aa, synthetic construct
  SITE 6: note = X is MetO2
CPFGCXKNWSWPIWC

SEQ ID NO: 58 - protein, 15 aa, synthetic construct
CPFGCMPNWSWPIWC

SEQ ID NO: 59 - protein, 15 aa, synthetic construct
CPFGCMQNWSWPIWC

SEQ ID NO: 60 - protein, 15 aa, synthetic construct
CPFGCMKNWSWPPWC

SEQ ID NO: 61 - protein, 15 aa, synthetic construct
  SITE 3: note = X is 2Pal
CPXGCMKNWSWPIWC

SEQ ID NO: 62 - protein, 15 aa, synthetic construct
  SITE 9: note = X is 1Nal
CPFGCMKNXSWPIWC

SEQ ID NO: 63 - protein, 15 aa, synthetic construct
  SITE 9: note = X is 2Nal
CPFGCMKNXSWPIWC

SEQ ID NO: 64 - protein, 15 aa, synthetic construct
  SITE 14: note = X is 2Nal
CPFGCMKNWSWPIXC

SEQ ID NO: 65 - protein, 15 aa, synthetic construct
  SITE 2: note = X is HyP
CXFGCMKNWSWPIWC

SEQ ID NO: 66 - protein, 15 aa, synthetic construct
  SITE 7: note = X is HArg
CPFDCMXNWSTPIWC

SEQ ID NO: 67 - protein, 15 aa, synthetic construct
  SITE 7: note = X is HArg
CPFDCMXKWSTPIWC

SEQ ID NO: 68 - protein, 15 aa, synthetic construct
  SITE 7: note = X is HArg
CPFDCMXNWSTPKWC

SEQ ID NO: 69 - protein, 15 aa, synthetic construct
  SITE 2: note = X is Pro(4NH)
  SITE 7: note = X is HArg
CXFDCMXNWSTPIWC

SEQ ID NO: 70 - protein, 15 aa, synthetic construct
CPFDCMKNWSTPIWC

SEQ ID NO: 71 - protein, 15 aa, synthetic construct
  SITE 7: note = X is HArg
CPFKCMXNWSTPIWC

SEQ ID NO: 72 - protein, 15 aa, synthetic construct
  SITE 7: note = X is HArg
CPFDCKXNWSTPIWC

SEQ ID NO: 73 - protein, 15 aa, synthetic construct
  SITE 7: note = X is HArg
CPFDCMXKWSTPIWC

SEQ ID NO: 74 - protein, 15 aa, synthetic construct
  SITE 2: note = X is Oxa
  SITE 7: note = X is HArg
CXFDCMXNWSTPIWC

SEQ ID NO: 75 - protein, 15 aa, synthetic construct
  SITE 7: note = X is HArg
  SITE 8: note = X is Thi
CPFDCMXXWSTPIWC

SEQ ID NO: 76 - protein, 15 aa, synthetic construct
  SITE 7: note = X is HArg
  SITE 8: note = X is 4ThiAz
CPFDCMXXWSTPIWC

SEQ ID NO: 77 - protein, 15 aa, synthetic construct
  SITE 7: note = X is HArg
  SITE 8: note = X is 124TriAz
CPFDCMXXWSTPIWC

SEQ ID NO: 78 - protein, 15 aa, synthetic construct
  SITE 7: note = X is HArg
  SITE 11: note = X is 124TriAz
CPFDCMXNWSXPIWC

SEQ ID NO: 79 - protein, 15 aa, synthetic construct
  SITE 7: note = X is HArg
  SITE 12: note = X is Oxa
CPFDCMXNWSTXIWC

SEQ ID NO: 80 - protein, 15 aa, synthetic construct
  SITE 3: note = X is DOPA
  SITE 7: note = X is HArg
CPXDCMXNWSTPIWC

SEQ ID NO: 81 - protein, 15 aa, synthetic construct
  SITE 7: note = X is HArg
  SITE 11: note = X is DOPA
CPFDCMXNWSXPIWC

SEQ ID NO: 82 - protein, 15 aa, synthetic construct
  SITE 7: note = X is HArg
  SITE 11: note = X is THP(SO2)
CPFDCMXNWSXPIWC

SEQ ID NO: 83 - protein, 15 aa, synthetic construct
  SITE 7: note = X is HArg
  SITE 13: note = X is THP(SO2)
CPFDCMXNWSTPXWC

SEQ ID NO: 84 - protein, 15 aa, synthetic construct
  SITE 7: note = X is HArg
  SITE 9: note = X is 5FTrp
CPFDCMXNXSTPIWC

SEQ ID NO: 85 - protein, 15 aa, synthetic construct
  SITE 7: note = X is HArg
  SITE 14: note = X is 5FTrp
CPFDCMXNWSTPIXC

SEQ ID NO: 86 - protein, 15 aa, synthetic construct
  SITE 7: note = X is HArg
  SITE 11: note = X is THP(O)
CPFDCMXNWSXPIWC

SEQ ID NO: 87 - protein, 15 aa, synthetic construct
  SITE 7: note = X is HArg
  SITE 13: note = X is THP(O)
CPFDCMXNWSTPXWC

SEQ ID NO: 88 - protein, 15 aa, synthetic construct
  SITE 2: note = X is 44DFP
  SITE 7: note = X is HArg
CXFDCMXNWSTPIWC

SEQ ID NO: 89 - protein, 15 aa, synthetic construct
  SITE 2: note = X is Oic
  SITE 7: note = X is HArg
CXFDCMXNWSTPIWC

SEQ ID NO: 90 - protein, 15 aa, synthetic construct
  SITE 7: note = X is HArg
CPFFCMXNWSTPIWC

SEQ ID NO: 91 - protein, 15 aa, synthetic construct
  SITE 7: note = X is HArg
CPFECMXNWSTPIWC

SEQ ID NO: 92 - protein, 15 aa, synthetic construct
  SITE 7: note = X is HArg
CPFQCMXNWSTPIWC

SEQ ID NO: 93 - protein, 15 aa, synthetic construct
  SITE 7: note = X is HArg
CPFLCMXNWSTPIWC

SEQ ID NO: 94 - protein, 15 aa, synthetic construct
  SITE 7: note = X is HArg
CPFSCMXNWSTPIWC

SEQ ID NO: 95 - protein, 15 aa, synthetic construct
  SITE 7: note = X is HArg
  SITE 10: note = X is HSer
CPFDCMXNWXTPIWC

SEQ ID NO: 96 - protein, 15 aa, synthetic construct
  SITE 7: note = X is HArg
  SITE 13: note = X is C5A
CPFDCMXNWSTPXWC

SEQ ID NO: 97 - protein, 15 aa, synthetic construct
  SITE 7: note = X is HArg
  SITE 13: note = X is Cpa
CPFDCMXNWSTPXWC

SEQ ID NO: 98 - protein, 15 aa, synthetic construct
  SITE 7: note = X is HArg
  SITE 13: note = X is Cha
CPFDCMXNWSTPXWC

SEQ ID NO: 99 - protein, 15 aa, synthetic construct
  SITE 6: note = X is HGln
  SITE 7: note = X is HArg
CPFDCXXNWSTPIWC

SEQ ID NO: 100 - protein, 15 aa, synthetic construct
  SITE 6: note = X is C5A
  SITE 7: note = X is HArg
CPFDCXXNWSTPIWC

SEQ ID NO: 101 - protein, 15 aa, synthetic construct
  SITE 7: note = Xaa is HArg
  SITE 9: note = X is Trp(Me)
CPFDCMXNXSTPIWC

SEQ ID NO: 102 - protein, 15 aa, synthetic construct
  SITE 5: note = X is NMeCys
  SITE 7: note = X is HArg
CPFDXMXNWSTPIWC

SEQ ID NO: 103 - protein, 14 aa, synthetic construct
  SITE 6: note = X is HArg
  SITE 10: note = X is NMeThr
CPFDCXNWSXPIWC

SEQ ID NO: 104 - protein, 15 aa, synthetic construct
  SITE 3: note = X is 1Nal
  SITE 7: note = X is HArg
CPXDCMXNWSTPIWC

SEQ ID NO: 105 - protein, 15 aa, synthetic construct
  SITE 3: note = X is 2Nal
  SITE 7: note = X is HArg
CPXDCMXNWSTPIWC

SEQ ID NO: 106 - protein, 15 aa, synthetic construct
  SITE 3: note = X is 44BPA
  SITE 7: note = X is HArg
CPXDCMXNWSTPIWC

SEQ ID NO: 107 - protein, 15 aa, synthetic construct
  SITE 7: note = X is HArg
CPFDCMXNWSTPPWC

SEQ ID NO: 108 - protein, 15 aa, synthetic construct
  SITE 7: note = X is HArg
  SITE 13: note = X is HyP
CPFDCMXNWSTPXWC

SEQ ID NO: 109 - protein, 15 aa, synthetic construct
  SITE 7: note = X is HArg
CPFDCLXNWSTPPWC

SEQ ID NO: 110 - protein, 15 aa, synthetic construct
  SITE 7: note = X is HArg
CPFDCLXNWSTPIWC

SEQ ID NO: 111 - protein, 15 aa, synthetic construct
  SITE 7: note = X is HArg
CPYDCMXNWSTPIWC

SEQ ID NO: 112 - protein, 15 aa, synthetic construct
  SITE 2: note = X is Aib
  SITE 7: note = X is HArg
CXFDCMXNWSTPIWC

SEQ ID NO: 113 - protein, 15 aa, synthetic construct
  SITE 2: note = X is Sar
  SITE 7: note = X is HArg
CXFDCMXNWSTPIWC

SEQ ID NO: 114 - protein, 15 aa, synthetic construct
  SITE 7: note = X is HArg
CPFRCMXNWSTPIWC

SEQ ID NO: 115 - protein, 15 aa, synthetic construct
  SITE 7: note = X is HArg
CPFDCMXNWSTPKWC

SEQ ID NO: 116 - protein, 15 aa, synthetic construct
  SITE 3: note = X is 1Nal
  SITE 7: note = X is HArg
  SITE 13: note = X is HyP
CPXDCMXNWSTPXWC

SEQ ID NO: 117 - protein, 15 aa, synthetic construct
  SITE 3: note = X is 1Nal
  SITE 7: note = X is HArg
  SITE 13: note = X is HyP
CPXDCMXHWSTPXWC

SEQ ID NO: 118 - protein, 15 aa, synthetic construct
  SITE 3: note = X is 1Nal
  SITE 7: note = X is HArg
  SITE 13: note = X is HyP
CPXDCMXDWSTPXWC

SEQ ID NO: 119 - protein, 15 aa, synthetic construct
  SITE 3: note = X is 1Nal
  SITE 7: note = X is HArg
CPXDCMXDWSTPIWC

SEQ ID NO: 120 - protein, 15 aa, synthetic construct
  SITE 3: note = X is 1Nal
  SITE 7: note = X is HArg
  SITE 13: note = X is HyP
CPXRCMXNWSTPXWC

SEQ ID NO: 121 - protein, 15 aa, synthetic construct
  SITE 3: note = X is 1Nal
  SITE 7: note = X is HArg
  SITE 13: note = X is HyP
CPXRCMXHWSTPXWC

SEQ ID NO: 122 - protein, 15 aa, synthetic construct
  SITE 7: note = X is NMeHArg
CPFDCMXNWSTPIWC

SEQ ID NO: 123 - protein, 15 aa, synthetic construct
  SITE 7: note = X is HArg
  SITE 8: note = X is NMeAsn
CPFDCMXXWSTPIWC

SEQ ID NO: 124 - protein, 15 aa, synthetic construct
  SITE 7: note = X is HArg
  SITE 11: note = X is NMeThr
CPFDCMXNWSXPIWC

SEQ ID NO: 125 - protein, 15 aa, synthetic construct
  SITE 7: note = X is HArg
  SITE 13: note = X is NMeIle
CPFDCMXNWSTPXWC

SEQ ID NO: 126 - protein, 15 aa, synthetic construct
  SITE 3: note = X is 1Nal
  SITE 7: note = X is HArg
  SITE 8: note = X is Cya
  SITE 13: note = X is HyP
CPXDCMXXWSTPXWC

SEQ ID NO: 127 - protein, 15 aa, synthetic construct
  SITE 3: note = X is 1Nal
  SITE 7: note = X is Cya
  SITE 13: note = X is HyP
CPXDCMXDWSTPXWC

SEQ ID NO: 128 - protein, 15 aa, synthetic construct
  SITE 3: note = X is 1Nal
  SITE 4: note = X is DCya
  SITE 7: note = X is HArg
  SITE 13: note = X is HyP
CPXXCMXDWSTPXWC

SEQ ID NO: 129 - protein, 15 aa, synthetic construct
  SITE 3: note = X is 1Nal
  SITE 7: note = X is HArg
  SITE 13: note = X is HyP
CPXDCMXDWDTPXWC

SEQ ID NO: 130 - protein, 15 aa, synthetic construct
  SITE 3: note = X is 2Nal
  SITE 7: note = X is HArg
  SITE 13: note = X is HyP
CPXDCMXDWSTPXWC

SEQ ID NO: 131 - protein, 15 aa, synthetic construct
  SITE 3: note = X is 1Nal
  SITE 7: note = X is HArg
  SITE 13: note = X is HyP
CPXDCMXDWTTPXWC

SEQ ID NO: 132 - protein, 15 aa, synthetic construct
  SITE 3: note = X is 1Nal
  SITE 7: note = X is HArg
  SITE 10: note = X is HSer
  SITE 13: note = X is HyP
CPXDCMXDWXTPXWC

SEQ ID NO: 133 - protein, 15 aa, synthetic construct
  SITE 3: note = X is 1Nal
  SITE 7: note = X is HArg
  SITE 13: note = X is HyP
CPXDCMXDWSTPXWC

SEQ ID NO: 134 - protein, 15 aa, synthetic construct
  SITE 3: note = X is 1Nal
  SITE 7: note = X is HArg
  SITE 13: note = X is HyP
CPXDCMXDWSSPXWC

SEQ ID NO: 135 - protein, 15 aa, synthetic construct
  SITE 3: note = X is 1Nal
  SITE 7: note = X is Agb
  SITE 13: note = X is HyP
CPXDCMXDWSTPXWC

SEQ ID NO: 136 - protein, 15 aa, synthetic construct
  SITE 3: note = X is 1Nal
  SITE 13: note = X is HyP
CPXDCMPDWSTPXWC

SEQ ID NO: 137 - protein, 15 aa, synthetic construct
  SITE 3: note = X is 1Nal
  SITE 7: note = X is HyP
  SITE 13: note = X is HyP
CPXDCMXDWSTPXWC

SEQ ID NO: 138 - protein, 15 aa, synthetic construct
  SITE 3: note = X is 1Nal
  SITE 7: note = X is HArg
  SITE 13: note = X is HyP
CPXRCMXDWSTPXWC

SEQ ID NO: 139 - protein, 15 aa, synthetic construct
  SITE 3: note = X is 1Nal
  SITE 7: note = X is HArg
  SITE 13: note = X is HyP
CPXRCMXDWDTPXWC

SEQ ID NO: 140 - protein, 15 aa, synthetic construct
  SITE 3: note = X is 2Nal
  SITE 7: note = X is HArg
  SITE 13: note = X is HyP
CPXRCMXDWSTPXWC

SEQ ID NO: 141 - protein, 15 aa, synthetic construct
  SITE 3: note = X is 1Nal
  SITE 7: note = X is HArg
  SITE 13: note = X is HyP
CPXRCMXDWTTPXWC

SEQ ID NO: 142 - protein, 15 aa, synthetic construct
  SITE 3: note = X is 1Nal
  SITE 7: note = X is HArg
  SITE 10: note = X is HSer
  SITE 13: note = X is HyP
CPXRCMXDWXTPXWC

SEQ ID NO: 143 - protein, 15 aa, synthetic construct
  SITE 3: note = X is 1Nal
  SITE 7: note = X is HArg
  SITE 13: note = X is HyP
CPXRCMXDWSTPXWC

SEQ ID NO: 144 - protein, 15 aa, synthetic construct
  SITE 3: note = X is 1Nal
  SITE 7: note = X is HArg
  SITE 13: note = X is HyP
CPXRCMXDWSSPXWC

SEQ ID NO: 145 - protein, 15 aa, synthetic construct
  SITE 3: note = X is 1Nal
  SITE 7: note = X is Agb
  SITE 13: note = X is HyP
CPXRCMXDWSTPXWC

SEQ ID NO: 146 - protein, 15 aa, synthetic construct
  SITE 3: note = X is 1Nal
  SITE 13: note = X is HyP
CPXRCMPDWSTPXWC

SEQ ID NO: 147 - protein, 15 aa, synthetic construct
  SITE 3: note = X is 1Nal
  SITE 7: note = X is HyP
  SITE 13: note = X is HyP
CPXRCMXDWSTPXWC

SEQ ID NO: 148 - protein, 15 aa, synthetic construct
  SITE 3: note = X is 1Nal
  SITE 7: note = X is HArg
CPXDCLXDWSTPIWC

SEQ ID NO: 149 - protein, 15 aa, synthetic construct
  SITE 3: note = X is 1Nal
  SITE 7: note = X is HArg
  SITE 13: note = X is HyP
CPXDCLXDWSTPXWC

SEQ ID NO: 150 - protein, 15 aa, synthetic construct
  SITE 3: note = X is 1Nal
  SITE 7: note = X is HArg
  SITE 13: note = X is HyP
CPXRCLXDWSTPXWC

SEQ ID NO: 151 - protein, 15 aa, synthetic construct
  SITE 3: note = X is 1Nal
  SITE 7: note = X is HArg
  SITE 13: note = X is HyP
CPXRCLXHWSTPXWC

SEQ ID NO: 152 - protein, 15 aa, synthetic construct
  SITE 3: note = X is 1Nal
  SITE 7: note = X is HArg
CPXRCMXDWSTPIWC

SEQ ID NO: 153 - protein, 15 aa, synthetic construct
  SITE 3: note = X is 1Nal
  SITE 4: note = X is DCya
  SITE 7: note = X is Cya
  SITE 13: note = X is HyP
CPXXCMXDWSTPXWC

SEQ ID NO: 154 - protein, 15 aa, synthetic construct
  SITE 3: note = X is 1Nal
  SITE 4: note = X is DCya
  SITE 7: note = X is HArg
  SITE 8: note = X is Cya
  SITE 13: note = X is HyP
CPXXCMXXWSTPXWC

SEQ ID NO: 155 - protein, 15 aa, synthetic construct
  SITE 3: note = X is 1Nal
  SITE 7: note = X is Cya
  SITE 8: note = X is Cya
  SITE 13: note = X is HyP
CPXDCMXXWSTPXWC

SEQ ID NO: 156 - protein, 15 aa, synthetic construct
  SITE 3: note = X is 1Nal
  SITE 7: note = X is HArg
  SITE 13: note = X is HyP
CPXKCMXDWSTPXWC

SEQ ID NO: 157 - protein, 15 aa, synthetic construct
  SITE 3: note = X is 1Nal
  SITE 13: note = X is HyP
CPXDCMKDWSTPXWC

SEQ ID NO: 158 - protein, 15 aa, synthetic construct
  SITE 3: note = X is 1Nal
  SITE 7: note = X is HArg
  SITE 13: note = X is HyP
CPXDCMXDWSTPXWC

SEQ ID NO: 159 - protein, 15 aa, synthetic construct
  SITE 7: note = X is HArg
  SITE 13: note = X is HyP
CPFGCMXDWSTPXWC

(19) United States
(12) Patent Application Publication
(10) Pub. No.: US 2024/0400608 A1
(43) Pub. Date: Dec. 5, 2024

(54) SYNTHESIS OF BICYCLE TOXIN CONJUGATES, AND INTERMEDIATES THEREOF

(71) Applicant: BricycleTx Limited, Cambridge (GB)

(72) Inventors: David Witty, Cambridge (GB); Darren Limb, Cambridge (GB); Byoung Joon Min, Cambridge (GB); Liwen He, Cambridge (GB); William J. Sanders, Cambridge (GB); Ernest Obinna Nnanabu, Cambridge (GB)

(73) Assignee: BricycleTx Limited, Cambridge (GB)

(21) Appl. No.: 18/688,590

(22) PCT Filed: Sep. 2, 2022
(86) PCT No.: PCT/GB2022/052249

§ 371 Date: Mar. 1, 2024

Related U.S. Application Data
(60) Provisional application No. 63/260,878, filed on Sep. 3, 2021.

(51) Int. Cl. C07K 1/02; C07K 1/24; C07K 1/30; C07K 14/435

(52) U.S. Cl. CPC C07K 1/026; C07K 1/24; C07K 1/30; C07K 14/43504

(57) ABSTRACT

The present invention relates to Bicycle toxin conjugates, methods for preparation, and methods of use for treating cancer.

### CROSS REFERENCE TO RELATED APPLICATIONS

This application claims priority to U.S. Provisional Application No. 63/260,878, filed Sep. 3, 2021, which is hereby incorporated herein by reference in its entirety.

### FIELD OF THE INVENTION

The present invention relates to methods for synthesizing Bicycle toxin conjugates (BTCs), for example, BT8009, comprising a constrained bicyclic peptide covalently linked to the potent anti-tubulin agent MMAE, and intermediates thereof.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVb3 (355 Å2) (Xiong et al. (2002), Science 296 (5565), 151-5) or the cyclic peptide inhibitor upain-1 binding to urokinase-type plasminogen activator (603 Å2; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher potential binding affinity. The reduced flexibility also leads to locking target-specific conformations, increasing binding specificity compared to linear peptides. This effect has been exemplified by a potent and selective inhibitor of matrix metalloproteinase 8, MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO2009/098450). Briefly, combinatorial libraries of linear peptides containing three cysteine residues and two regions of six random amino acids (Cys-(Xaa)6-Cys-(Xaa)6-Cys) were displayed on phage and cyclised by covalently linking the cysteine side chains to a small molecule (tris-(bromomethyl)benzene).

### SUMMARY OF THE INVENTION

The present invention provides Bicycle toxin conjugates, and methods of preparation. In some embodiments, a Bicycle toxin conjugate of the invention comprises a constrained bicyclic peptide covalently linked to the potent anti-tubulin agent MMAE. In some embodiments, a Bicycle toxin conjugate comprises a constrained bicyclic peptide that binds with high affinity and specificity to Nectin-4.

In some embodiments, the present invention provides a Bicycle toxin conjugate of formula I:

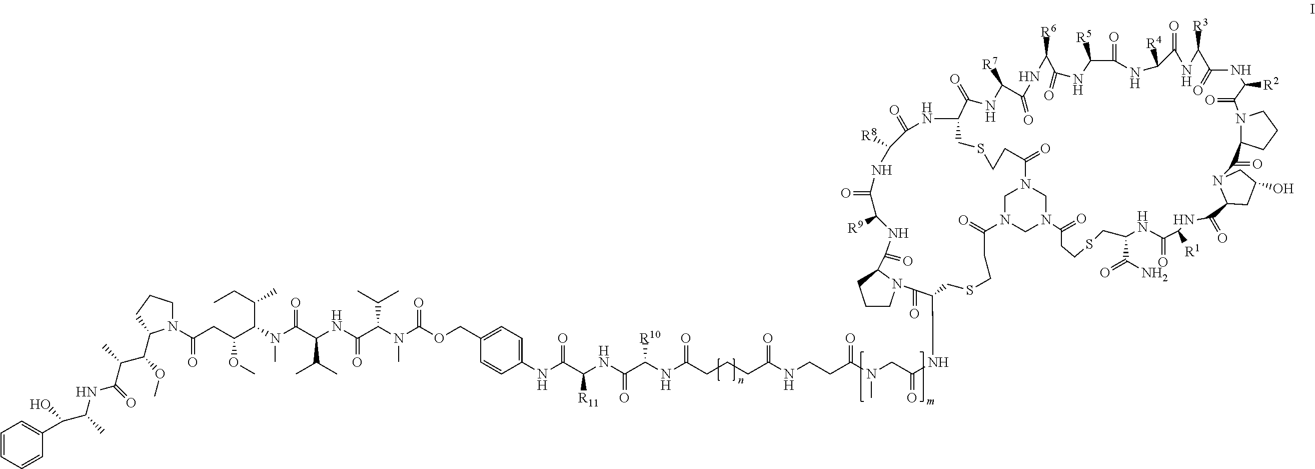

or a pharmaceutically acceptable salt thereof, wherein each of R¹, R², R³, R⁴, R⁵, R⁶, R⁷, R⁸, R⁹, R¹⁰, R¹¹, m, and n is as defined below and described in embodiments herein, both singly and in combination.

In some embodiments, the present invention provides a method for preparing a Bicycle toxin conjugate of the invention, or a synthetic intermediate thereof, according to schemes and steps as described herein.

In some embodiments, the present invention provides a method for preventing and/or treating cancers as described herein comprising administering to a patient a Bicycle toxin conjugate of the invention.

In some embodiments, the present invention provides a synthetic intermediate, or a composition thereof, useful for preparing a Bicycle toxin conjugate of the invention.

### BRIEF DESCRIPTION OF THE DRAWINGS

FIG. 1 depicts the Factorial Regression: +56(%) versus TFA (%), DTT (%). The CenterPt is shown in FIG. 1 which depicts the normal plot of the effects for the +56 impurity.

FIG. 2 depicts the Pareto chart of the effects for the +56 impurity.

FIG. 3 depicts the Factorial Regression: +163(%) versus TFA (%), DTT (%). The CenterPt is shown in FIG. 3 which depicts the normal plot of the effects for the +163 impurity.

FIG. 4 depicts the Pareto chart of the effects for the +163 impurity.

FIG. 5 depicts the response optimization for cleavage cocktails.

### DETAILED DESCRIPTION OF THE INVENTION

### 1. General Description of Certain Aspects of the Invention

A number of Bicycle toxin conjugates, and the methods of synthesis thereof, are described in International Patent Application No. PCT/GB2019/051740 (International Publication No. WO 2019/243832), the entirety of which is incorporated herein by reference. For example, a Bicycle toxin conjugate BCY8245 (BT8009) is described as synthesized by: step 1) solid phase synthesis of Fmoc-Val-Cit; step 2) Fmoc deprotection; step 3) amide formation with monomethyl glutaric acid; step 4) cleavage of glutaryl-Val-Cit methyl ester off the resin under mild acidic conditions; step 5) amide formation at the C terminus with p-amino benzyl alcohol; step 6) formation of a p-nitrophenylcarbamate using bis(4-nitrophenyl)carbonate; step 7) treatment with MMAE to form the p-amino phenyl carbamate; step 8) hydrolysis of the glutaryl methyl ester to form the acid; step 9) activation of the acid and treatment with N-hydroxy succinimide to form the activated NHS ester; step 10) treatment of the NHS ester with BCY8234 in the presence of base (DIEA) in DMA to form BCY8245 followed by standard reverse phase purification using a C18 semi-preparative column (TFA condition) and lyophilization to obtain pure Bicycle toxin conjugate BCY8245 (BT8009).

It has now been found that the number of steps in the synthetic route can be reduced and the impurity profile and yield can be improved by treatment of Val-Cit-PAB-MMAE with glutaric anhydride and direct amide formation with the resulting acid and BCY8234.

The improved BCY8245 process includes, but is not limited to, the following features:
simplified two-step process;
improved yields (44% yield over two steps and 96.9% LC purity);
use of 1 equivalent of gvcMMAE/TBTU in the coupling step (second step) to reduce an identified RRT 0.93 impurity; and
optimized filtration and column purification steps.

Additionally, improvements in the synthesis of the bicyclic peptide BCY8234 were made.

The improved BCY8234 process includes, but is not limited to, the following features:
reduction in the amount of formed aspartimide impurities;
optimization of the deblocking cocktail comprising 3% oxyma in 10% piperidine/DMF;
use of DITU in the coupling reactions to help suppress cysteine oxidation;
use of sarcosine dipeptide derivatives in the sarcosine couplings;
use of higher loading (>0.8 mmol/g) resin;
reduction of TATA to 1.3 eq., reduction of the reaction time to 4 hours, and reduction in the ACN content to 20% in the bicyclic peptide forming step;
identification of the optimal pH values for column loading (pH=6.8) and longer term crude product storage (pH=4.5); and
desalting of the purified TFA salt followed by lyophilization for improved long-term stability.

Accordingly, in one aspect, the present invention provides a Bicycle toxin conjugate of formula I:

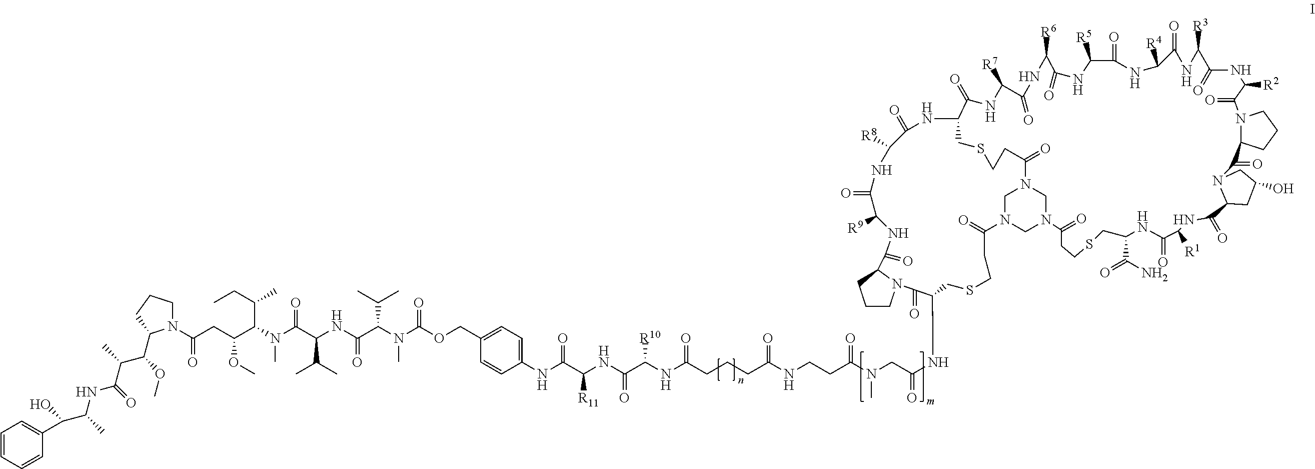

or a pharmaceutically acceptable salt thereof,
wherein:
each of R¹, R², R, R⁴, R⁵, R⁶, R⁷, R⁸, R⁹, R¹⁰, and R¹¹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
m is 0, 1, 2, 3, 4, 5, 6, 7, 8, 9, 10, 11, 12, 13, 14, or 15; and
n is 0, 1, or 2.

In another aspect, the present invention provides a method for preparing a Bicycle toxin conjugate of formula I, or a salt thereof. In certain embodiments, the present compounds are generally prepared according to Scheme I set forth below, wherein each of the variables, reagents, intermediates, and reaction steps is as defined below and described in embodiments herein, both singly and in combination.

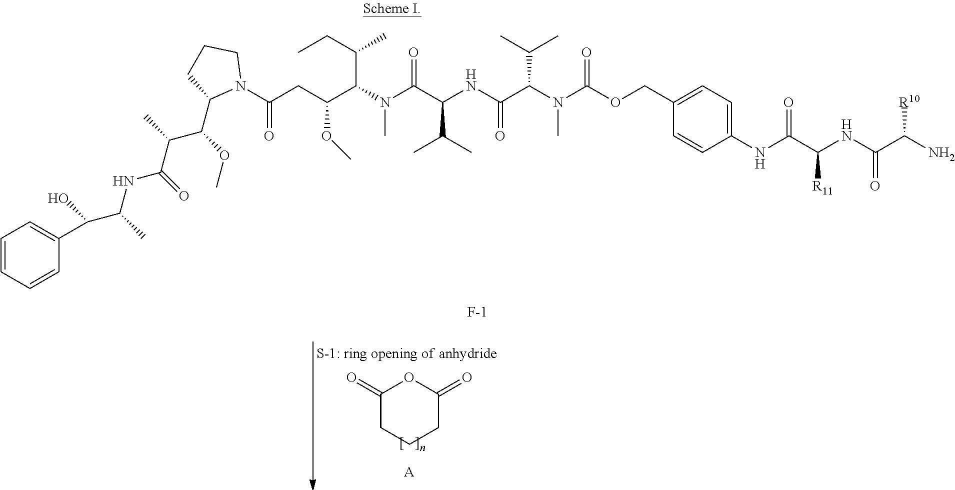

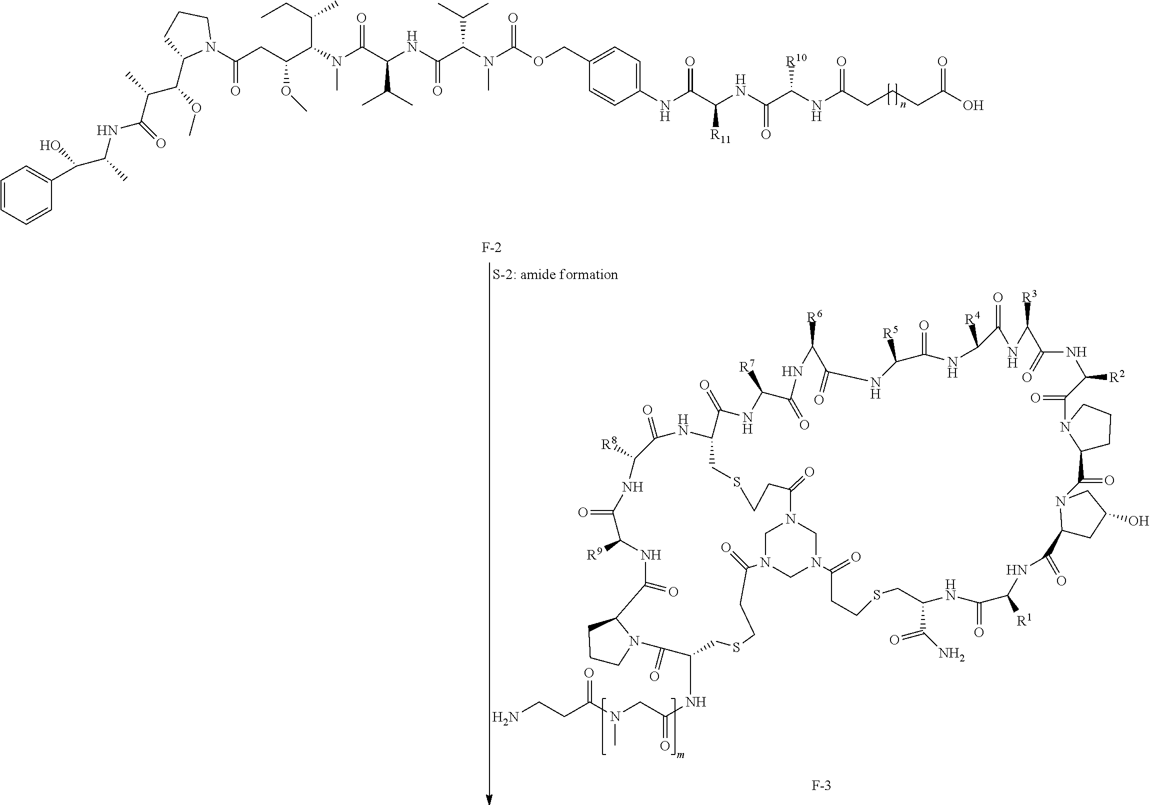

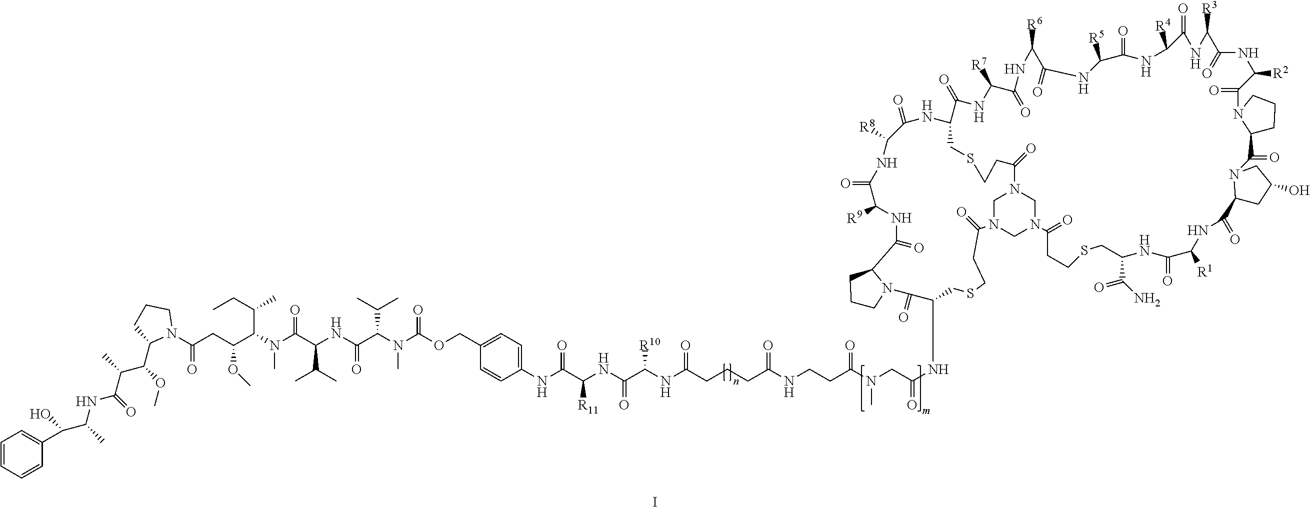

The variables R¹, R², R³, R⁴, R⁵, R⁶, R⁷, R⁸, R⁹, R¹⁰, R¹¹, m, and n is as defined above and in classes and subclasses as described herein.

In one aspect, the present invention provides methods for preparing Bicycle toxin conjugates (BTCs) of formula I from homochiral starting materials with high enantiomeric and diastereomeric purity according to the steps depicted in Scheme I, above. In compounds of the present formulae, R¹, R², R³, R⁴, R⁵, R⁶, R⁷, R⁸, R⁹, R¹⁰, and R¹¹ are as defined as above for compounds of formula I and are each independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In compounds of the present formulae, m is as defined as above for compounds of formula I and is 0, 1, 2, 3, 4, 5, 6, 7, 8, 9, 10, 11, 12, 13, 14, or 15.

In compounds of the present formulae, n is as defined as above for compounds of formula I and is 0, 1, or 2.

At step S-1, a fragment of formula F-1 is coupled to an anhydride of formula A to form a fragment of formula F-2, via a ring-opening addition to the anhydride.

At step S-2, a fragment of F-2 is coupled to a fragment of F-3, to form a compound of formula I via amide formation. Amide formation can be accomplished with a wide variety of coupling agents known in the art such as, but not limited to:
N,N-Dicyclohexylcarbodiimide (DCC);
N,N′-Diisopropylcarbodiimide (DIC);
N-(3-Dimethylaminopropyl)-N-ethylcarbodiimide (EDC);
N-[(Dimethylamino)-1H-1,2,3-triazolo-[4,5-b]pyridin-1-ylmethylene]-N-methylmethanaminium hexafluorophosphate N-oxide (HATU);
N,N,N′,N′-Tetramethyl-O-(1H-benzotriazol-1-yl)uronium hexafluorophosphate (HBTU);
O-(1H-6-Chlorobenzotriazole-1-yl)-1,1,3,3-tetramethyluronium hexafluorophosphate (HCTU);
(Benzotriazol-1-yloxy)tripyrrolidinophosphonium hexafluorophosphate (PyBOP);
(7-Azabenzotriazol-1-yloxy)tripyrrolidinophosphonium hexafluorophosphate (PyAOP);
Bromotripyrrolidinophosphonium hexafluorophosphate (PyBrOP);
Benzotriazole-1-yl-oxy-tris-(dimethylamino)-phosphonium hexafluorophosphate (BOP);
Bis(2-oxo-3-oxazolidinyl)phosphinic chloride (BOP-Cl);
3-(diethoxyphosphoryloxy)-1,2,3-benzotriazin-4(3H)-one (DEPBT);
2,4,6-Tripropyl-1,3,5,2,4,6-trioxatriphosphorinane-2,4,6-trioxide (T3P);
1-[Bis(dimethylamino)methylene]-1H-1,2,3-triazolo[4,5-b]pyridinium 3-oxide tetrafluoroborate (TATU);
N,N,N′,N′-Tetramethyl-O-(benzotriazol-1-yl)uronium tetrafluoroborate (TBTU);
2-(endo-5-norbornene-2.3-dicarboxylimide)-1,1,3,3-tetramethyluronium tetrafluoroborate (TNTU);
O-[(Ethoxycarbonyl)cyanomethylenamino]-N,N,N′,N′-tetramethyluronium tetrafluoroborate (TOTU);
O-(2-Oxo-1(2H)pyridyl)-N,N,N′,N′-tetramethyluronium tetrafluoroborate (TPTU);
N,N,N′,N′-Tetramethyl-O—(N-succinimidyl)uronium tetrafluoroborate (TSTU); or
O-(3,4-Dihydro-4-oxo-1,2,3-benzotriazin-3-yl)-N,N,N′,N′-tetramethyluronium tetrafluoroborate (TDBTU).

In another aspect, the present invention provides a method for preparing Fragment F-3, or a salt thereof. In certain embodiments, the present compounds are generally prepared according to Scheme II set forth below, wherein each of the variables, reagents, intermediates, and reaction steps is as defined below and described in embodiments herein, both singly and in combination.

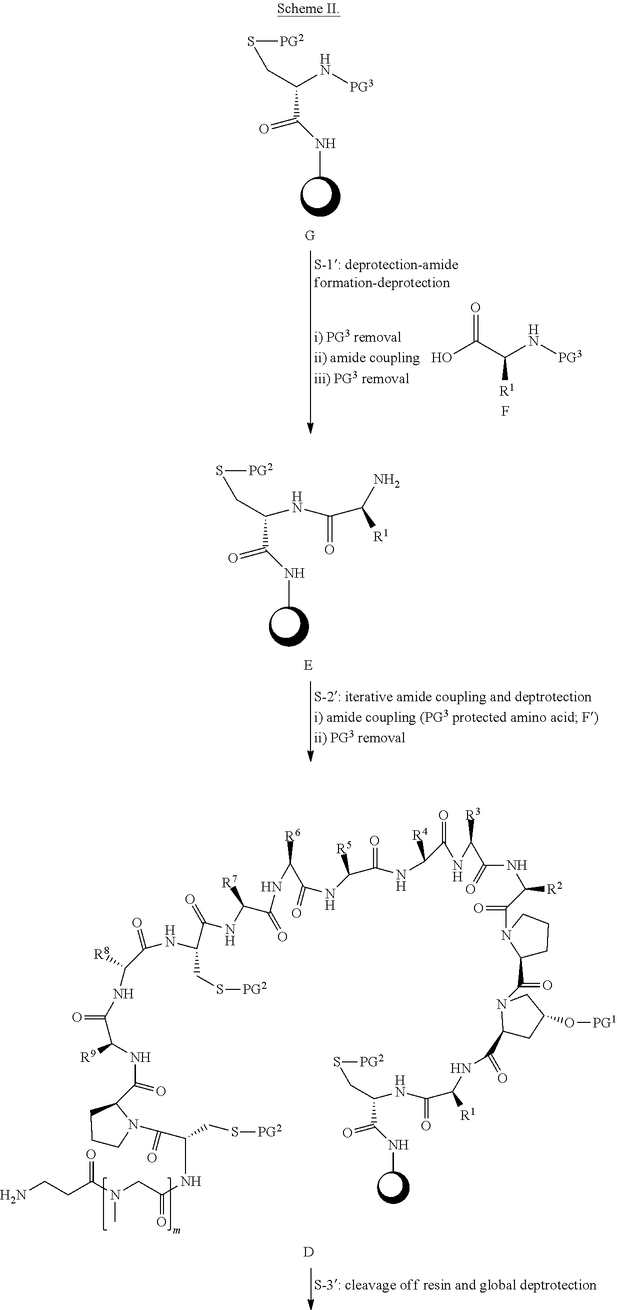

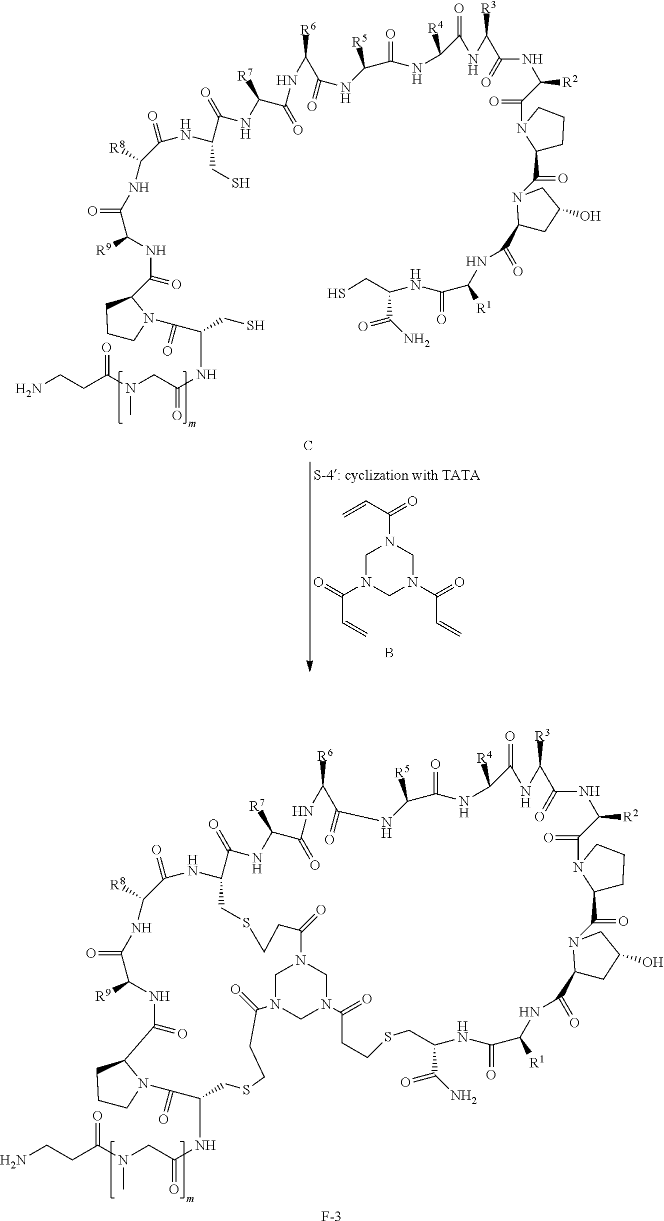

In one aspect, the present invention provides methods for preparing a fragment of formula F-3 in enantiomerically enriched form according to the steps depicted in Scheme II, above. In compounds of the present formulae, R¹, R², R³, R⁴, R⁵, R⁶, R⁷, R⁸, R⁹, R¹⁰, and R¹¹ are as defined as above for compounds of formula I and are each independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In compounds of the present formulae, m is as defined as above for compounds of formula I and is 0, 1, 2, 3, 4, 5, 6, 7, 8, 9, 10, 11, 12, 13, 14, or 15.

In compounds of the present formulae, n is as defined as above for compounds of formula I and is 0, 1, or 2.

At step S-1′, a compound of formula G is deprotected to remove the nitrogen protecting group PG³ and then coupled to a protected amino acid of formula F followed by PG³ removal to form a compound of formula E, via amide formation.

One of ordinary skill in the art would recognize that the PG³ may be removed using a variety of conditions. In some embodiments, PG³ removal may be accomplished by treatment with 20% piperidine in DMF (deblocking step). In some embodiments, PG³ removal may be followed by a wash cycle with DMF prior to a coupling/recoupling step.

At step S-2′, a compound of formula E is iteratively coupled to a PG³ protected amino acid followed by PG³ removal, to form a compound of formula D via amide formation. Amide formation can be accomplished with a wide variety of coupling agents known in the art such as, but not limited to DCC, DIC, EDC, HATU, HBTU, HCTU, PyBOP, PyAOP, PyBrOP, BOP, BOP-Cl, DEPBT, T3P, TATU, TBTU, TNTU, TOTU, TPTU, TSTU, or TDBTU. One of ordinary skill in the art would recognize that amide formation can be accomplished with the above-referenced coupling agents.

In some embodiments, amide formation is accomplished using DIC/oxyma to afford a compound of formula D.

At step S-3′, a compound of formula D is a) cleaved from the solid phase resin and b) globally deprotected (i.e. removal of the indicated PG² and PG¹ protecting groups and any additional protecting groups on the R¹, R², R³, R⁴, R⁵, R⁶, R⁷, R⁸, R⁹, R¹⁰, and R¹¹ groups) to afford a compound of formula C. One of ordinary skill in the art would recognize that cleavage from the solid phase and global deprotection can be accomplished by treatment with acid. One of ordinary skill in the art would also recognize that cleavage from the solid phase and global deprotection can be accomplished in a single step by treatment with a TFA cocktail comprising an acid such as TFA, and cation trapping agents including but not limited to DTT, TIS and NH₄I in a solvent such as water.

At step S-4′, a compound of formula C is cyclized on to compound B (TATA) to afford a compound of formula F-3. One of ordinary skill in the art would recognize that the reaction proceeds via three Michael additions of the cysteine residues in the compound of formula C to TATA and can be accomplished under basic conditions to afford the cyclic product.

Each PG¹ group of formula D is independently a suitable alcohol protecting group. Suitable alcohol protecting groups are well known in the art and include those described in detail in Protecting Groups in Organic Synthesis, T. W. Greene and P. G. M. Wuts, 4^{th} Edition, John Wiley & Sons, 2006, the entirety of which is incorporated herein by reference. Suitable alcohol protecting groups, taken with the —O— moiety to which they are attached, include, but are not limited to, ethers, substituted methyl ethers, substituted ethyl ethers, substituted benzyl ethers, and the like. Examples of PG¹ groups of formula D include t-butyl (tBu), methyl, ethyl, methoxymethyl, tetrahydrofuranyl, allyl, benzyl (Bn), acetate, 2-hydroxyethyl and the like. In certain embodiments, the PG¹ group in compounds of formula D is t-butyl (tBu), methyl, acetate, or ethyl. In other embodiments, the PG¹ group in compounds of formula D is t-butyl (tBu).

Each PG² group of formulae D, E, and G is independently a suitable thiol protecting group. Suitable thiol protecting groups are well known in the art and include those described in detail in Protecting Groups in Organic Synthesis, T. W. Greene and P. G. M. Wuts, 4^{th} Edition, John Wiley & Sons, 2006, the entirety of which is incorporated herein by reference. Suitable thiol protecting groups, taken with the —S— moiety to which they are attached, include, but are not limited to, ethers, substituted methyl ethers, substituted ethyl ethers, substituted benzyl ethers, and the like. Examples of PG² groups of formulae D, E, and G include t-butyl (tBu), methyl, ethyl, methoxymethyl, tetrahydrofuranyl, allyl, benzyl (Bn), diphenylmethyl, triphenylmethyl (Tr), adamantyl and the like. In certain embodiments, the PG² group in compounds of formulae D, E, and G is triphenylmethyl (Tr), t-butyl (tBu), methyl, diphenylmethyl, or adamantyl. In other embodiments, the PG² group in compounds of formulae D, E, and G is triphenylmethyl (Tr).

Each PG³ group of formulae F and F′ is independently a suitable amino protecting group. Suitable amino protecting groups are well known in the art and include those described in detail in Protecting Groups in Organic Synthesis, T. W. Greene and P. G. M. Wuts, 4^{th} Edition, John Wiley & Sons, 2006, the entirety of which is incorporated herein by reference. Suitable amino protecting groups, taken with the —NH— moiety to which they are attached, include, but are not limited to, aralkylamines, carbamates, allyl amines, amides, and the like. Examples of PG³ groups of formulae F and F′ include t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, pivaloyl and the like. In certain embodiments, the PG³ group in compounds of formulae F and F′ is t-butyloxycarbonyl, ethyloxycarbonyl, fluorenylmethylcarbonyl (Fmoc), or acetyl. In other embodiments, the PG³ group in compounds of formulae F and F′ is fluorenylmethylcarbonyl (Fmoc).

One of ordinary skill in the art will recognize that the iterative amide coupling and deprotection protocol with homochiral building blocks described herein can be adapted to provide compounds of formulae E, D, C, and F-3 in high enantiomeric and diastereomeric purity. In certain embodiments, one diastereomer of a compound of formulae E, D, C, and F-3 is formed substantially free from other stereoisomers. “Substantially free,” as used herein, means that the compound is made up of a significantly greater proportion of one diastereomer. In other embodiments, at least about 98% by weight of a desired diastereomer is present. In still other embodiments of the invention, at least about 99% by weight of a desired diastereomer is present. Such diastereomers may be isolated from diastereomeric mixtures by any method known to those skilled in the art, including high performance liquid chromatography (HPLC) and crystallization, or prepared by methods described herein.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of each of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

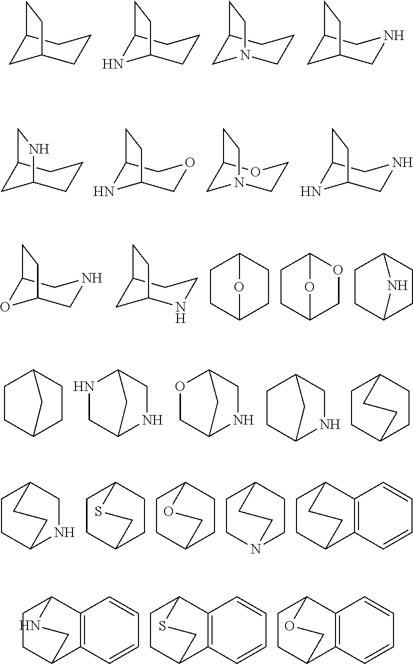

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one triple bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

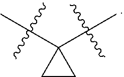 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —N(R^{∘})C(NR^{∘})N(R^{∘})₂; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄₀S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{•} (or the ring formed by taking two independent occurrences of R^{•} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR*, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Additionally, pharmaceutically acceptable salts are described in detail in Pharmaceutical Salts: Properties, Selection, and Use, 2nd Revised Edition, (2011), P. Heinrich Stahl (Editor), Camille G. Wermuth (Editor), (ISBN: 978-3-906-39051-2), the entirety of which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, (C₁₋₆ alkyl)sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, condition, or disorder, to treat, diagnose, prevent, and/or delay the onset of the disease, condition, or disorder. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, condition, or disorder is the amount that alleviates, ameliorates, relieves, inhibits, prevents, delays onset of, reduces severity of and/or reduces incidence of one or more symptoms or features of the disease, condition, or disorder.

The terms “treat” or “treating,” as used herein, refers to partially or completely alleviating, inhibiting, delaying onset of, preventing, ameliorating and/or relieving a disease or disorder, or one or more symptoms of the disease or disorder. As used herein, the terms “treatment,” “treat,” and “treating” refer to partially or completely alleviating, inhibiting, delaying onset of, preventing, ameliorating and/or relieving a disease or disorder, or one or more symptoms of the disease or disorder, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Bicycle toxin conjugate BT8009 has the structure shown below, and a preparation of BT8009 (BCY8245) is described in WO 2019/243832, the entirety of which is hereby incorporated herein by reference.

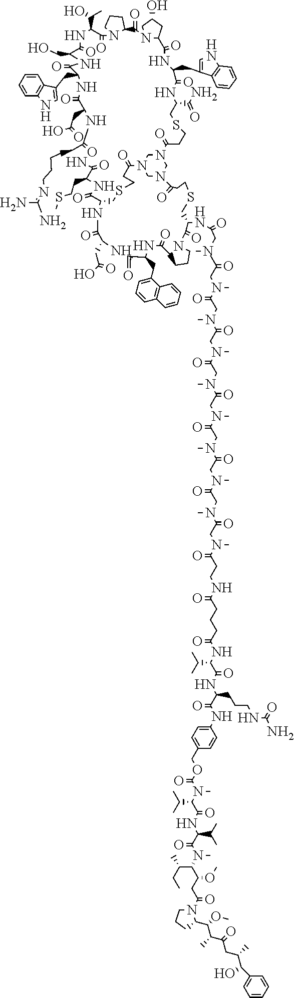

### 3. Description of Synthesis of Bicycle Toxin Conjugate of Formula I and Relevant Intermediates

In some embodiments, the present invention provides a method for preparing a Bicycle toxin conjugate of formula I according to Scheme I, wherein each of the variables, reagents, intermediates, and reaction steps is as defined below and described in embodiments herein, both singly and in combination.

The compound of formula I in Scheme I comprises a constrained bicyclic peptide that binds with high affinity and specificity to Nectin-4. In some embodiments, the bicyclic peptide is selected from those described in International Patent Application No. PCT/GB2019/051740 (International Publication No. WO 2019/243832), the entirety of which is incorporated herein by reference. In some embodiments, the bicyclic peptide is a peptide covalently bound to a molecular scaffold. In some embodiments, the bicyclic peptide comprises a peptide having three cysteine residues (referred as Cᵢ, Cᵢᵢ, and Cᵢᵢᵢ in the sequences below), which are capable of forming covalent bonds to a molecular scaffold. In some embodiments, the bicyclic peptide comprises a peptide Cᵢ-P/A/Hyp-F/Y-G/A-Cᵢᵢ-X₁-X₂-X₃-W/1-Nal/2-Nal-S/A-X₄-P-I/D/A-W/1-Nal/2-Nal-Cᵢᵢᵢ (SEQ ID NO: 1);
Cᵢ-W/A-P-L-D/S-S/D-Y-W-Cᵢᵢ-X₅-R-I-Cᵢᵢᵢ (SEQ ID NO: 2);
Cᵢ-V-T-T-S-Y-D-Cᵢᵢ-F/W-L/V-H/R/T-L-L/G-G/Q/H-Cᵢᵢᵢ (SEQ ID NO: 3);
Cᵢ-X₆-X₇-X₅-Cᵢ-X₉-X₁₀-X₁₁-X₂-X₁₃-X₁₄-X₁₅-X₁₆-X₁₇-Cᵢᵢᵢ (SEQ ID NO: 4); and
Cᵢ-W/A/Y-P/A-L-D/S/A-S/D/P/A-Y-W/1-Nal-Cᵢᵢ-X₅-R/HArg/A-I-Cᵢᵢᵢ (SEQ ID NO: 5); wherein:
   X₁-X₅ represent any amino acid residue, including modified and non-natural amino acids; X₆ represents: Gly; Pro or a non-natural derivative of Pro selected from azetidine (Aze), hydroxyproline (HyP), 4-amino-proline (Pro(4NH)), oxazolidine-4-carboxylic acid (Oxa), octahydroindolecarboxylic acid (Oic) or 4,4-difluoroproline (4,4-DFP); Ala or a non-natural derivative of Ala selected from aminoisobutyric acid (Aib); or Sarcosine (Sar);
   X₇ represents: Phe or a non-natural derivative of Phe selected from 3-methyl-phenylalanine (3MePhe), 4-methyl-phenylalanine (4MePhe), homophenylalanine (HPhe), 4,4-biphenylalanine (4,4-BPA) or 3,4-diydroxy-phenylalanine (DOPA); Tyr; or Ala or a non-natural derivative of Ala selected from 1-naphthylalanine (1-Nal), 2-naphthylalanine (2-Nal) or 2-pyridylalanine (2Pal);
   X₈ represents: Gly; Ala; Asp; Lys or a non-natural derivative of Lys selected from acetyl-lysine (KAc or Lys(Ac)); Phe; Glu; Gln; Leu; Ser; Arg; or cysteic acid (Cya); X₉ is either absent or represents: Met or a non-natural derivative of Met selected from methionine sulfone (Met(O2)); Gln or a non-natural derivative of Gln selected from homoglutamine (HGln); Leu or a non-natural derivative of Leu selected from homoleucine (HLeu) or norleucine (Nle); Lys; Ile; t-butyl-alanine (tBuAla); or homoserine-methyl (HSe(Me)); X₁₀ represents: Pro; Lys or a non-natural derivative of Lys selected from acetyl-lysine (KAc or Lys(Ac)); Arg or a non-natural derivative of Arg selected from 2-amino-4-guanidinobutyric acid (Agb), homoarginine (HArg) or N-methyl-homoarginine; Glu; Ser; Asp; Gln; Ala; hydroxyproline (HyP); or cysteic acid (Cya);
   X₁₁ represents: Asn or a non-natural derivative of Asn selected from N-methyl-asparagine; Thr; Asp; Gly; Ser; His; Ala or a non-natural derivative of Ala selected from thienyl-alanine (Thi), 2-(1,2,4-triazol-1-yl)-alanine (1,2,4-TriAz) or Beta-(4-thiazolyl)-alanine (4ThiAz); Lys; or cysteic acid (Cya);
   X₁₂ represents: Trp or a non-natural derivative of Trp selected from azatryptophan (AzaTrp), 5-fluoro-L-tryptophan (5FTrp) or methyl-tryptophan (TrpMe); or Ala or a non-natural derivative of Ala selected from 1-naphthyl alanine (1-Nal) or 2-naphthyl alanine (2-Nal);
   X₁₃ represents: Ser or a non-natural derivative of Ser selected from homoserine (HSer); Ala; Asp; or Thr;
   X₁₄ represents: Trp or a non-natural derivative of Trp selected from azatryptophan (AzaTrp); Ser; Ala or a non-natural derivative of Ala selected from 2-(1,2,4-triazol-1-yl)-alanine (1,2,4-TriAz), 1-naphthyl alanine (1-Nal) or 2-naphthyl alanine (2-Nal); Asp; Phe or a non-natural derivative of Phe selected from 3,4-diydroxy-phenylalanine (DOPA); Tyr; Thr or a non-natural derivative of Thr selected from N-methyl-threonine; tetrahydropyran-4-propanoic acid (THP(O)); or dioxo-4-tetrahydrothiopyranylacetic acid (THP(SO2));
   X₁₅ represents Pro or a non-natural derivative of Pro selected from azetidine (Aze), pipecolic acid (Pip) or oxazolidine-4-carboxylic acid (Oxa);
   X₁₆ represents: Ile or a non-natural derivative of Ile selected from N-methyl-isoleucine (NMeIle); Ala or a non-natural derivative of Ala selected from 3-cyclohexyl-alanine (Cha) or cyclopropyl-alanine (Cpa); Pro or a non-natural derivative of Pro selected from hydroxyproline (HyP); Asp; Lys; cyclopentyl-glycine (C5A); tetrahydropyran-4-propanoic acid (THP(O)); or dioxo-4-tetrahydrothiopyranylacetic acid (THP(SO2));
   X₁₇ represents: Trp or a non-natural derivative of Trp selected from azatryptophan (AzaTrp) or 5-fluoro-L-tryptophan (5FTrp); Phe; Tyr; 1-naphthyl alanine (1-Nal); or 2-naphthyl alanine (2-Nal);
   Hyp represents hydroxyproline, 1-Nal represents 1-naphthyl alanine, 2-Nal represents 2-naphthyl alanine, HArg represents homoarginine and Cᵢ, Cᵢᵢ and Cᵢᵢᵢ represent first, second and third cysteine residues, respectively or a pharmaceutically acceptable salt thereof.

In some embodiments, the bicyclic peptide comprises a peptide selected from the following:

(SEQ ID NO: 6)
CPFGCMETWSWPIWC;
(SEQ ID NO: 7)
CPFGCMRGWSWPIWC;
(SEQ ID NO: 8)
CPFGCMSGWSWPIWC;
(SEQ ID NO: 9)
CPFGCMEGWSWPIWC;
(SEQ ID NO: 10)
CPFGCMEDWSWPIWC;
(SEQ ID NO: 11)
CPFGCMPGWSWPIWC;
(SEQ ID NO: 12)
CPFGCMKSWSWPIWC;
(SEQ ID NO: 13)
CPFGCMKTWSWPIWC;
(SEQ ID NO: 14)
CPFGCMKGWSWPIWC;
(SEQ ID NO: 15)
CPFGCQEHWSWPIWC;
(SEQ ID NO: 16)
CPFGCIKSWSWPIWC;
(SEQ ID NO: 17)
CPFGCQEDWSWPIWC;
(SEQ ID NO: 18)
CPFGCMSDWSWPIWC;
(SEQ ID NO: 19)
CPFGCM[HArg]NWSWPIWC;
(SEQ ID NO: 20)
CPFGCM[K(Ac)]NWSWPIWC;
(SEQ ID NO: 21)
CPFGCM[K(Ac)]SWSWPIW;
(SEQ ID NO: 22)
CPFGC[Nle]KSWSWPIWC;
(SEQ ID NO: 23)
CPFGCM[HArg]SWSWPIWC;
(SEQ ID NO: 24)
CPFGCM[dK]SWSWPIWC;
(SEQ ID NO: 25)
CP[dA]GCMKNWSWPIWC;
(SEQ ID NO: 26)
CPF[dA]CMKNWSWPIWC;
(SEQ ID NO: 27)
CPFGCM[dA]NWSWPIWC;
(SEQ ID NO: 28)
CPFGCMK[dA]WSWPIWC;
(SEQ ID NO: 29)
CPFGCMKN[dA]SWPIWC;
(SEQ ID NO: 30)
CPFGCMKNWSWP[dA]WC;
(SEQ ID NO: 31)
C[dA]FGCMKNWSWPIWC;
(SEQ ID NO: 32)
CPFGC[tBuAla]KNWSWPIWC;
(SEQ ID NO: 33)
CPFGC[HLeu]KNWSWPIWC;
(SEQ ID NO: 34)
CPFGCMKNWSWPI[1Nal]C;
(SEQ ID NO: 35)
CPF[dD]CM[HArg]NWSWPIWC;
(SEQ ID NO: 36)
CPF[dA]CM[HArg]NWSWPIWC;
(SEQ ID NO: 37)
CP[3MePhe]GCMKNWSWPIWC;
(SEQ ID NO: 38)
CP[4MePhe]GCMKNWSWPIWC;
(SEQ ID NO: 39)
CP[HPhe]GCMKNWSWPIWC;
(SEQ ID NO: 40)
CPF[dD]CMKNWSWPIWC;
(SEQ ID NO: 41)
CPFGC[Hse(Me)]KNWSWPIWC;
(SEQ ID NO: 42)
CPFGCMKN[AzaTrp]SWPIWC;
(SEQ ID NO: 43)
CPFGCMKNWSFPIWC;
(SEQ ID NO: 44)
CPFGCMKNWSYPIWC;
(SEQ ID NO: 45)
CPFGCMKNWS[1Nal]PIWC;
(SEQ ID NO: 46)
CPFGCMKNWS[2Nal]PIWC;
(SEQ ID NO: 47)
CPFGCMKNWS[AzaTrp]PIWC;
(SEQ ID NO: 48)
CPFGCMKNWSW[Aze]IWC;
(SEQ ID NO: 49)
CPFGCMKNWSW[Pip]IWC;
(SEQ ID NO: 50)
CPFGCMKNWSWPIFC;
(SEQ ID NO: 51)
CPFGCMKNWSWPIYC;
(SEQ ID NO: 52)
CPFGCMKNWSWPI[AzaTrp]C;
(SEQ ID NO: 53)
CGFGCMKNWSWPIWC;
(SEQ ID NO: 54)
C[Aze]FGCMKNWSWPIWC;
(SEQ ID NO: 55)
CPF[K(Ac)]CMKNWSWPIWC;
(SEQ ID NO: 56)
CPFGCLKNWSWPIWC;
(SEQ ID NO: 57)
CPFGC[MetO2]KNWSWPIWC;
(SEQ ID NO: 58)
CPFGCMPNWSWPIWC;
(SEQ ID NO: 59)
CPFGCMQNWSWPIWC;
(SEQ ID NO: 60)
CPFGCMKNWSWPPWC;
(SEQ ID NO: 61)
CP[2Pal]GCMKNWSWPIWC;
(SEQ ID NO: 62)
CPFGCMKN[1Nal]SWPIWC;
(SEQ ID NO: 63)
CPFGCMKN[2Nal]SWPIWC;
(SEQ ID NO: 64)
CPFGCMKNWSWPI[2Nal]C;
(SEQ ID NO: 65)
C[HyP]FGCMKNWSWPIWC;
(SEQ ID NO: 66)
CPF[dD]CM[HArg]NWSTPIWC;
(SEQ ID NO: 67)
CPF[dD]CM[HArg][dK]WSTPIWC;
(SEQ ID NO: 68)
CPF[dD]CM[HArg]NWSTPKWC;
(SEQ ID NO: 69)
C[Pro(4NH)]F[dD]CM[HArg]NWSTPIWC;
(SEQ ID NO: 70)
CPF[dD]CMKNWSTPIWC;
(SEQ ID NO: 71)
CPF[dK]CM[HArg]NWSTPIWC;
(SEQ ID NO: 72)
CPF[dD]CK[HArg]NWSTPIWC;
(SEQ ID NO: 73)
CPF[dD]CM[HArg]KWSTPIWC;
(SEQ ID NO: 74)
C[Oxa]F[dD]CM[HArg]NWSTPIWC;
(SEQ ID NO: 75)
CPF[dD]CM[HArg][Thi]WSTPIWC;
(SEQ ID NO: 76)
CPF[dD]CM[HArg][4ThiAz]WSTPIWC;
(SEQ ID NO: 77)
CPF[dD]CM[HArg][124TriAz]WSTPIWC;
(SEQ ID NO: 78)
CPF[dD]CM[HArg]NWS[124TriAz]PIWC;
(SEQ ID NO: 79)
CPF[dD]CM[HArg]NWST[Oxa]IWC;
(SEQ ID NO: 80)
CP[DOPA][dD]CM[HArg]NWSTPIWC;
(SEQ ID NO: 81)
CPF[dD]CM[HArg]NWS[DOPA]PIWC;
(SEQ ID NO: 82)
CPF[dD]CM[HArg]NWS[THP(SO2)]PIWC;
(SEQ ID NO: 83)
CPF[dD]CM[HArg]NWSTP[THP(SO2)]WC;
(SEQ ID NO: 84)
CPF[dD]CM[HArg]N[5FTrp]STPIWC;
(SEQ ID NO: 85)
CPF[dD]CM[HArg]NWSTPI[5FTrp]C;
(SEQ ID NO: 86)
CPF[dD]CM[HArg]NWS[THP(O)]PIWC;
(SEQ ID NO: 87)
CPF[dD]CM[HArg]NWSTP[THP(O)]WC;
(SEQ ID NO: 88)
C[44DFP]F[dD]CM[HArg]NWSTPIWC;
(SEQ ID NO: 89)
C[Oic]F[dD]CM[HArg]NWSTPIWC;
(SEQ ID NO: 90)
CPF[dF]CM[HArg]NWSTPIWC;
(SEQ ID NO: 91)
CPF[dE]CM[HArg]NWSTPIWC;
(SEQ ID NO: 92)
CPF[dQ]CM[HArg]NWSTPIWC;
(SEQ ID NO: 93)
CPF[dL]CM[HArg]NWSTPIWC;
(SEQ ID NO: 94)
CPF[dS]CM[HArg]NWSTPIWC;
(SEQ ID NO: 95)
CPF[dD]CM[HArg]NW[HSer]TPIWC;
(SEQ ID NO: 96)
CPF[dD]CM[HArg]NWSTP[C5A]WC;
(SEQ ID NO: 97)
CPF[dD]CM[HArg]NWSTP[Cpa]WC;
(SEQ ID NO: 98)
CPF[dD]CM[HArg]NWSTP[Cha]WC;
(SEQ ID NO: 99)
CPF[dD]C[HGln][HArg]NWSTPIWC;
(SEQ ID NO: 100)
CPF[dD]C[C5A][HArg]NWSTPIWC;
(SEQ ID NO: 101)
CPF[dD]CM[HArg]N[Trp(Me)]STPIWC;
(SEQ ID NO: 102)
CPF[dD][NMeCys]M[HArg]NWSTPIWC;
(SEQ ID NO: 103)
CPF[dD]C[HArg]NWS[NMeThr]PIWC;
(SEQ ID NO: 104)
CP[1Nal][dD]CM[HArg]NWSTPIWC;
(SEQ ID NO: 105)
CP[2Nal][dD]CM[HArg]NWSTPIWC;
(SEQ ID NO: 106)
CP[44BPA][dD]CM[HArg]NWSTPIWC;
(SEQ ID NO: 107)
CPF[dD]CM[HArg]NWSTPPWC;
(SEQ ID NO: 108)
CPF[dD]CM[HArg]NWSTP[HyP]WC;
(SEQ ID NO: 109)
CPF[dD]CL[HArg]NWSTPPWC;
(SEQ ID NO: 110)
CPF[dD]CL[HArg]NWSTPIWC;
(SEQ ID NO: 111)
CPY[dD]CM[HArg]NWSTPIWC;
(SEQ ID NO: 112)
C[Aib]F[dD]CM[HArg]NWSTPIWC;
(SEQ ID NO: 113)
C[Sar]F[dD]CM[HArg]NWSTPIWC;
(SEQ ID NO: 114)
CPF[dR]CM[HArg]NWSTPIWC;
(SEQ ID NO: 115)
CPF[dD]CM[HArg]NWSTPKWC;
(SEQ ID NO: 116)
CP[1Nal][dD]CM[HArg]NWSTP[HyP]WC;
(SEQ ID NO: 117)
CP[1Nal][dD]CM[HArg]HWSTP[HyP]WC:
(SEQ ID NO: 118)
CP[1Nal][dD]CM[HArg]DWSTP[HyP]WC;
(SEQ ID NO: 119)
CP[1Nal][dD]CM[HArg]DWSTPIWC;
(SEQ ID NO: 120)
CP[1Nal][dR]CM[HArg]NWSTP[HyP]WC;
(SEQ ID NO: 121)
CP[1Nal][dR]CM[HArg]HWSTP[HyP]WC;
(SEQ ID NO: 122)
CPF[dD]CM[NMeHArg]NWSTPIWC;
(SEQ ID NO: 123)
CPF[dD]CM[HArg][NMeAsn]WSTPIWC;
(SEQ ID NO: 124)
CPF[dD]CM[HArg]NWS[NMeThr]PIWC;
(SEQ ID NO: 125)
CPF[dD]CM[HArg]NWSTP[NMeIle]WC;
(SEQ ID NO: 126)
CP[1Nal][dD]CM[HArg][Cya]WSTP[HyP]WC;
(SEQ ID NO: 127)
CP[1Nal][dD]CM[Cya]DWSTP[HyP]WC;
(SEQ ID NO: 128)
CP[1Nal][DCya]CM[HArg]DWSTP[HyP]WC;
(SEQ ID NO: 129)
CP[1Nal][dD]CM[HArg]DWDTP[HyP]WC;
(SEQ ID NO: 130)
CP[2Nal][dD]CM[HArg]DWSTP[HyP]WC;
(SEQ ID NO: 131)
CP[1Nal][dD]CM[HArg]DWTTP[HyP]WC;
(SEQ ID NO: 132)
CP[1Nal][dD]CM[HArg]DW[HSer]TP[HyP]WC;
(SEQ ID NO: 133)
CP[1Nal][dD]CM[HArg]DW[dS]TP[HyP]WC;
(SEQ ID NO: 134)
CP[1Nal][dD]CM[HArg]DWSSP[HyP]WC;
(SEQ ID NO: 135)
CP[1Nal][dD]CM[Agb]DWSTP[HyP]WC;
(SEQ ID NO: 136)
CP[1Nal][dD]CMPDWSTP[HyP]WC;
(SEQ ID NO: 137)
CP[1Nal][dD]CM[HyP]DWSTP[HyP]WC;
(SEQ ID NO: 138)
CP[1Nal][dR]CM[HArg]DWSTP[HyP]WC;
(SEQ ID NO: 139)
CP[1Nal][dR]CM[HArg]DWDTP[HyP]WC;
(SEQ ID NO: 140)
CP[2Nal][dR]CM[HArg]DWSTP[HyP]WC;
(SEQ ID NO: 141)
CP[1Nal][dR]CM[HArg]DWTTP[HyP]WC;
(SEQ ID NO: 142)
CP[1Nal][dR]CM[HArg]DW[HSer]TP[HyP]WC;
(SEQ ID NO: 143)
CP[1Nal][dR]CM[HArg]DW[dS]TP[HyP]WC;
(SEQ ID NO: 144)
CP[1Nal][dR]CM[HArg]DWSSP[HyP]WC;
(SEQ ID NO: 145)
CP[1Nal][dR]CM[Agb]DWSTP[HyP]WC;
(SEQ ID NO: 146)
CP[1Nal][dR]CMPDWSTP[HyP]WC;
(SEQ ID NO: 147)
CP[1Nal][dR]CM[HyP]DWSTP[HyP]WC;
(SEQ ID NO: 148)
CP[1Nal][dD]CL[HArg]DWSTPIWC;
(SEQ ID NO: 149)
CP[1Nal][dD]CL[HArg]DWSTP[HyP]WC;
(SEQ ID NO: 150)
CP[1Nal][dR]CL[HArg]DWSTP[HyP]WC;
(SEQ ID NO: 151)
CP[1Nal][dR]CL[HArg]HWSTP[HyP]WC;
(SEQ ID NO: 152)
CP[1Nal][dR]CM[HArg]DWSTPIWC;
(SEQ ID NO: 153)
CP[1Nal][DCya]CM[Cya]DWSTP[HyP]WC;
(SEQ ID NO: 154)
CP[1Nal][DCya]CM[HArg][Cya]WSTP[HyP]WC;
(SEQ ID NO: 155)
CP[1Nal][dD]CM[Cya][Cya]WSTP[HyP]WC;
(SEQ ID NO: 156)
CP[1Nal][dK]CM[HArg]DWSTP[HyP]WC;
(SEQ ID NO: 157)
CP[1Nal][dD]CMKDWSTP[HyP]WC;
(SEQ ID NO: 158)
CP[1Nal][dD]CM[HArg]D[dW]STP[HyP][dW]C;
and
(SEQ ID NO: 159)
CPFGCM[HArg]DWSTP[HyP]WC.

In some embodiments, the bicyclic peptide is:

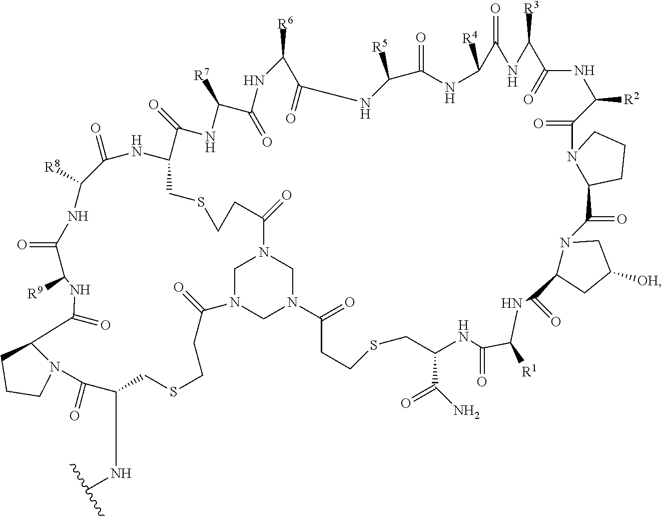

wherein each of R¹, R², R³, R⁴, R⁵, R⁶, R⁷, R, and R⁹ is as independently defined below and described in embodiments herein, both singly and in combination.

In some embodiments, each of R, R², R, R⁴, R⁵, R⁶, R⁷, R, and R⁹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In certain embodiments, R¹ is hydrogen or optionally substituted C₁₋₆ aliphatic. In certain embodiments, R¹ is

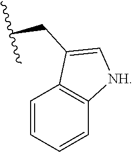

In certain embodiments, R² is hydrogen or optionally substituted C₁₋₆ aliphatic. In certain embodiments, R² is

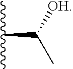

In certain embodiments, R³ is hydrogen or optionally substituted C₁₋₆ aliphatic. In certain embodiments, R³ is

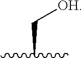

In certain embodiments, R⁴ is hydrogen or optionally substituted C₁₋₆ aliphatic. In certain embodiments, R⁴ is

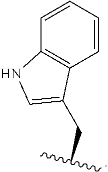

In certain embodiments, R⁵ is hydrogen or optionally substituted C₁₋₆ aliphatic. In certain embodiments, R⁵ is

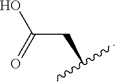

In certain embodiments, R⁶ is hydrogen or optionally substituted C₁₋₆ aliphatic. In certain embodiments, R⁶ is

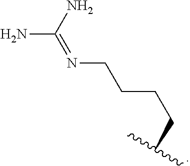

In certain embodiments, R⁷ is hydrogen or optionally substituted C₁₋₆ aliphatic. In certain embodiments, R⁷ is

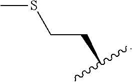

In certain embodiments, R⁸ is hydrogen or optionally substituted C₁₋₆ aliphatic. In certain embodiments, R⁸ is

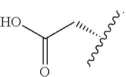

In certain embodiments, R⁹ is hydrogen or optionally substituted C₁₋₆ aliphatic. In certain embodiments, R⁹ is

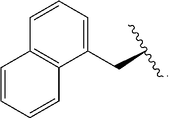

In certain embodiments, R¹⁰ is hydrogen or optionally substituted C₁₋₆ aliphatic. In certain embodiments, R¹⁰ is

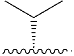

In certain embodiments, R¹¹ is hydrogen or optionally substituted C₁₋₆ aliphatic. In certain embodiments, R¹¹ is

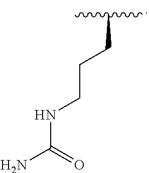

In some embodiments, the Bicycle toxin conjugate of formula I is:

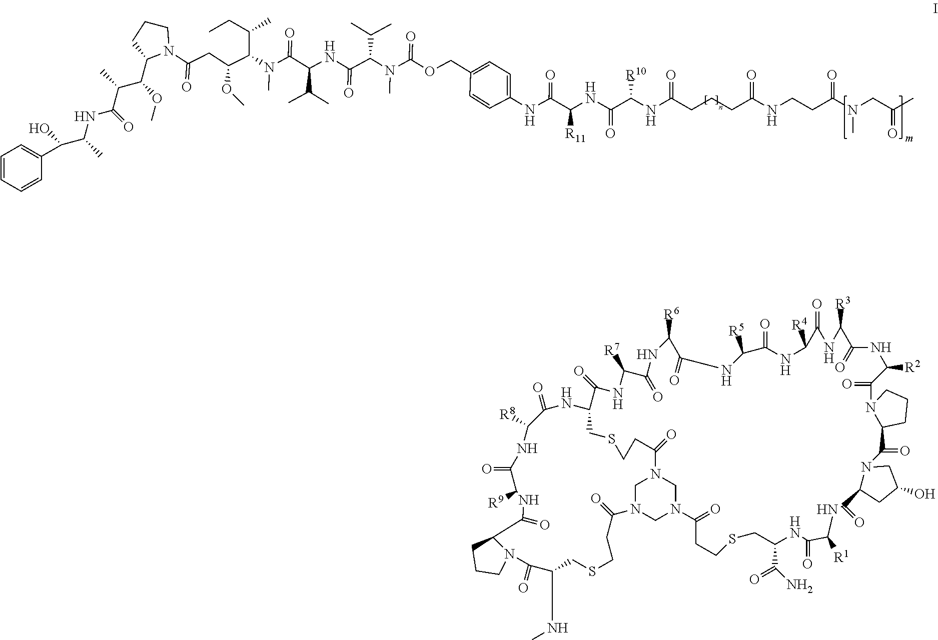

or a pharmaceutically acceptable salt thereof,
wherein:
each of R¹, R², R³, R⁴, R⁵, R⁶, R⁷, R⁸, R⁹, R¹⁰, and R¹¹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
m is 0, 1, 2, 3, 4, 5, 6, 7, 8, 9, 10, 11, 12, 13, 14, or 15; and
n is 0, 1, or 2.

In certain embodiments, R¹ is hydrogen or optionally substituted C₁₋₆ aliphatic. In certain embodiments, R¹ is

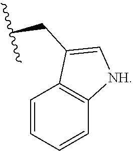

In certain embodiments, R² is hydrogen or optionally substituted C₁₋₆ aliphatic. In certain embodiments, R² is

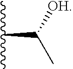

In certain embodiments, R³ is hydrogen or optionally substituted C₁₋₆ aliphatic. In certain embodiments, R³ is

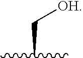

In certain embodiments, R⁴ is hydrogen or optionally substituted C₁₋₆ aliphatic. In certain embodiments, R⁴ is

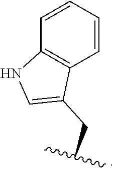

In certain embodiments, R⁵ is hydrogen or optionally substituted C₁₋₆ aliphatic. In certain embodiments, R⁵ is

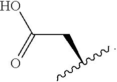

In certain embodiments, R⁶ is hydrogen or optionally substituted C₁₋₆ aliphatic. In certain embodiments, R⁶ is

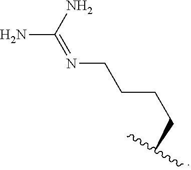

In certain embodiments, R⁷ is hydrogen or optionally substituted C₁₋₆ aliphatic. In certain embodiments, R⁷ is

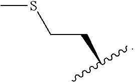

In certain embodiments, R⁸ is hydrogen or optionally substituted C₁₋₆ aliphatic. In certain embodiments, R⁸ is

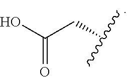

In certain embodiments, R⁹ is hydrogen or optionally substituted C₁₋₆ aliphatic. In certain embodiments, R⁹ is

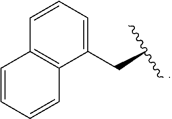

In certain embodiments, R¹⁰ is hydrogen or optionally substituted C₁₋₆ aliphatic. In certain embodiments, R¹⁰ is

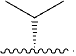

In certain embodiments, R¹¹ is hydrogen or optionally substituted C₁₋₆ aliphatic. In certain embodiments, R¹¹ is

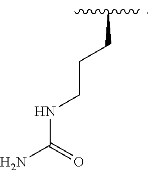

In certain embodiments, m is 0, 1, 2, 3, 4, 5, 6, 7, 8, 9, 10, 11, 12, 13, 14, or 15.

In certain embodiments, m is 0. In certain embodiments, m is 1. In certain embodiments, m is 2. In certain embodiments, m is 3, In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15.

In certain embodiments, n is 0, 1, or 2.

In certain embodiments, n is 0. In certain embodiments, n is 1. In certain embodiments, n is 2.

Each of R¹, R², R, R⁴, R⁵, R⁶, R⁷, R⁸, R⁹, R¹⁰, and R¹¹ in Scheme I is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In certain embodiments, R¹ is hydrogen or optionally substituted C₁₋₆ aliphatic. In certain embodiments, R¹ is

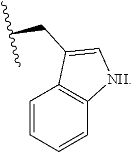

In certain embodiments, R² is hydrogen or optionally substituted C₁₋₆ aliphatic. In certain embodiments, R² is

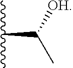

In certain embodiments, R³ is hydrogen or optionally substituted C₁₋₆ aliphatic. In certain embodiments, R³ is

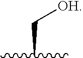

In certain embodiments, R⁴ is hydrogen or optionally substituted C₁₋₆ aliphatic. In certain embodiments, R⁴ is

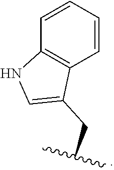

In certain embodiments, R⁵ is hydrogen or optionally substituted C₁₋₆ aliphatic. In certain embodiments, R⁵ is

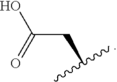

In certain embodiments, R⁶ is hydrogen or optionally substituted C₁₋₆ aliphatic. In certain embodiments, R⁶ is

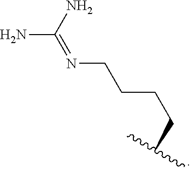

In certain embodiments, R⁷ is hydrogen or optionally substituted C₁₋₆ aliphatic. In certain embodiments, R⁷ is

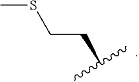

In certain embodiments, R⁸ is hydrogen or optionally substituted C₁₋₆ aliphatic. In certain embodiments, R⁸ is

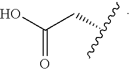

In certain embodiments, R⁹ is hydrogen or optionally substituted C₁₋₆ aliphatic. In certain embodiments, R⁹ is

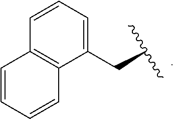

In certain embodiments, R¹⁰ is hydrogen or optionally substituted C₁₋₆ aliphatic. In certain embodiments, R¹⁰ is

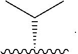

In certain embodiments, R¹¹ is hydrogen or optionally substituted C₁₋₆ aliphatic. In certain embodiments, R¹¹ is

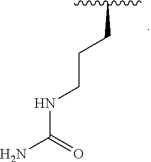

In Scheme I, m is 0, 1, 2, 3, 4, 5, 6, 7, 8, 9, 10, 11, 12, 13, 14, or 15.

In certain embodiments, m is 0. In certain embodiments, m is 1. In certain embodiments, m is 2. In certain embodiments, m is 3, In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15.

In Scheme I, n is 0, 1, or 2.

In certain embodiments, n is 0. In certain embodiments, n is 1. In certain embodiments, n is 2.

Each of R¹, R², R³, R⁴, R⁵, R⁶, R⁷, R, and R⁹ in Scheme II is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In certain embodiments, R¹ is hydrogen or optionally substituted C₁₋₆ aliphatic. In certain embodiments, R¹ is

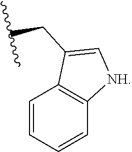

In certain embodiments, R² is hydrogen or optionally substituted C₁₋₆ aliphatic. In certain embodiments, R² is

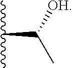

In certain embodiments, R³ is hydrogen or optionally substituted C₁₋₆ aliphatic. In certain embodiments, R³ is

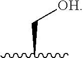

In certain embodiments, R⁴ is hydrogen or optionally substituted C₁₋₆ aliphatic. In certain embodiments, R⁴ is

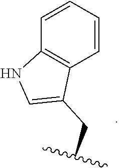

In certain embodiments, R⁵ is hydrogen or optionally substituted C₁₋₆ aliphatic. In certain embodiments, R⁵ is

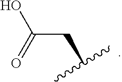

In certain embodiments, R⁶ is hydrogen or optionally substituted C₁₋₆ aliphatic. In certain embodiments, R⁶ is

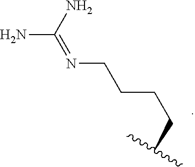

In certain embodiments, R⁷ is hydrogen or optionally substituted C₁₋₆ aliphatic. In certain embodiments, R⁷ is

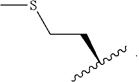

In certain embodiments, R⁸ is hydrogen or optionally substituted C₁₋₆ aliphatic. In certain embodiments, R⁸ is

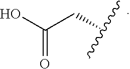

In certain embodiments, R⁹ is hydrogen or optionally substituted C₁₋₆ aliphatic. In certain embodiments, R⁹ is

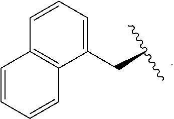

In Scheme II, m is 0, 1, 2, 3, 4, 5, 6, 7, 8, 9, 10, 11, 12, 13, 14, or 15.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15.

Fragment F-3 can be prepared or isolated in general by synthetic and/or semi-synthetic methods known to those skilled in the art for analogous compound (for example, as described in WO 2019/243832, the entire content of which is incorporated herein by reference) and by methods described in detail in the Examples, herein.

In some embodiments, fragment F-3 in Scheme I is:

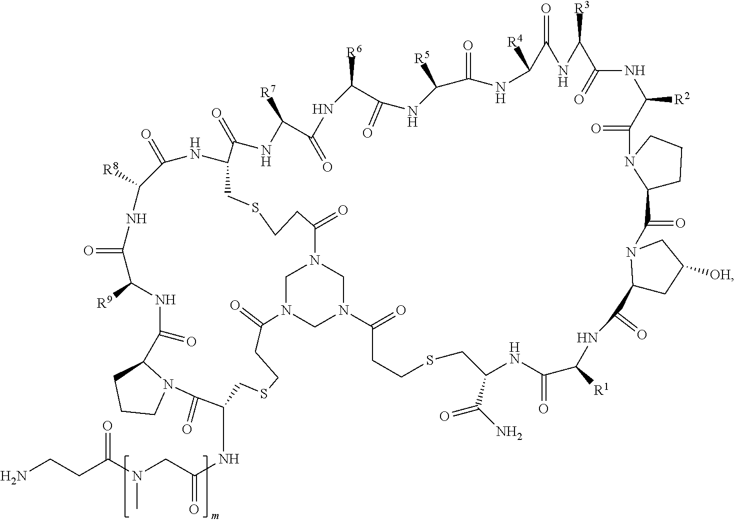

or a salt thereof, wherein each of R¹, R², R³, R⁴, R⁵, R⁶, R⁷, R⁸, R⁹, and m is as defined below and described in embodiments herein, both singly and in combination.

In some embodiments, fragment F-3 in Scheme I is:

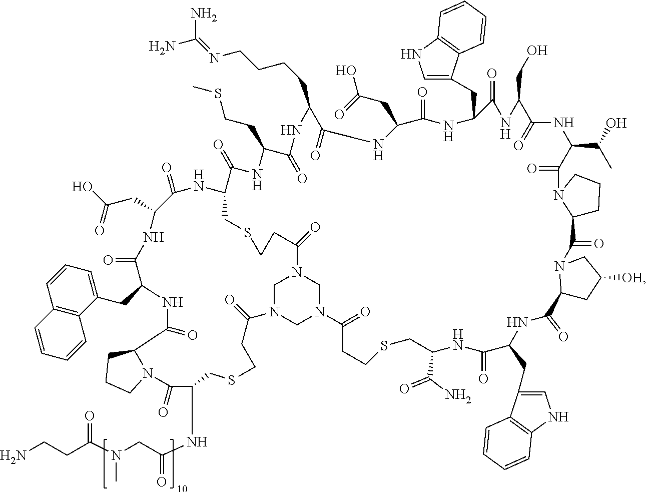

or a salt thereof.

In some embodiments, fragment F-2 in Scheme I is:

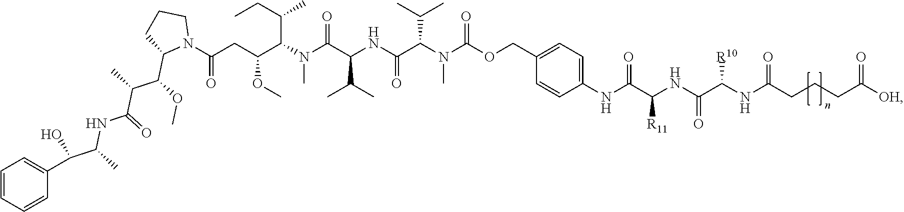

or a salt thereof, wherein each of R¹⁰, R¹¹, and n is as defined below and described in embodiments herein, both singly and in combination.

In some embodiments, fragment F-2 in Scheme I is:

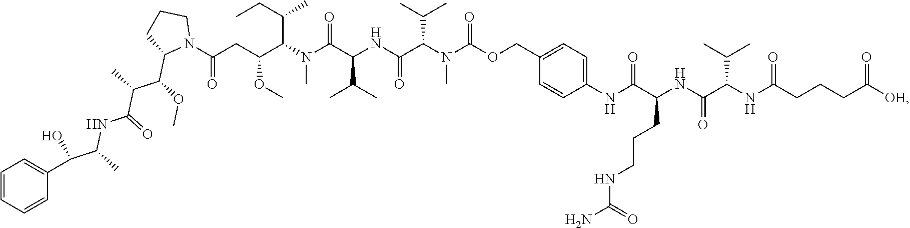 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

or a salt thereof.

In some embodiments, fragment F-3 in Scheme I is:

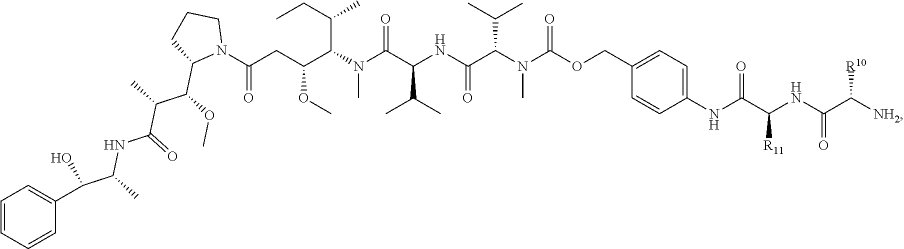

or a salt thereof, wherein each of R¹⁰ and R¹¹ is as defined below and described in embodiments herein, both singly and in combination.

In some embodiments, fragment F-3 in Scheme I is:

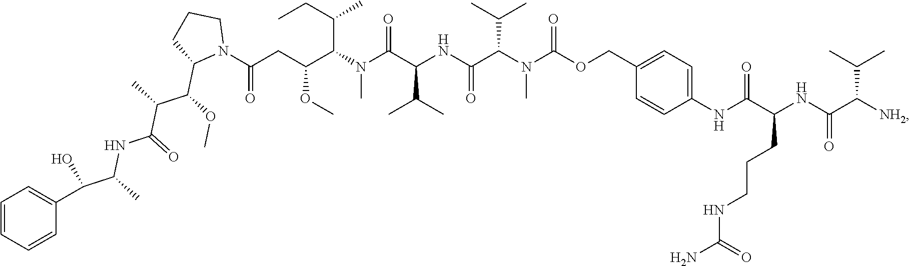 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

or a salt thereof.

At Step S-1 (amide formation via ring opening of anhydride), fragment F-1, or a salt thereof, is coupled to compound A, or a salt thereof, to form fragment F-2, or a salt thereof. Suitable coupling reactions are well known to one of ordinary skill in the art and typically involve an activated ester derivative (e.g., an anhydride) such that treatment with an amine moiety results in the formation of an amide bond. The coupling reaction is typically carried out in the presence of an excess of a base. In some embodiments, the base is a tertiary amine base. In some embodiments, the tertiary amine base is triethylamine. In some embodiments, the base is a tertiary amine base. In some embodiments, the tertiary amine base is N,N-Diisopropylethylamine (DIPEA). The coupling reaction may be carried out in a suitable solvent that solubilizes all of the reagents. In some embodiments, the solvent is a dipolar aprotic solvent. In some embodiments, the dipolar aprotic solvent is N,N-dimethylacetamide (DMA). In some embodiments, the dipolar aprotic solvent is dimethyl sulfoxide (DMSO), N,N-dimethylformamide (DMF), acetone, ethyl acetate, hexamethylphosphoramide (HMPA) or N,N′-dimethylpropyleneurea (DMPU). In some embodiments, the reaction mixture is mixed with an acidic water solution to precipitate out fragment F-2, or a salt thereof. In some embodiments, the reaction mixture is mixed with an acidic brine solution to precipitate out fragment F-2, or a salt thereof. In some embodiments, the brine solution is a 13% brine solution. In some embodiments, the brine solution is a saturated brine solution. In some embodiments, fragment F-2, or a salt thereof, obtained by precipitation and filtration is of a purity of about 80% or higher. In some embodiments, fragment F-2, or a salt thereof, obtained by precipitation and filtration is of a purity of about 82%, 84%, 86%, 88%, 90%, 92%, 94%, 96%, or 98%. In some embodiments, fragment F-2, or a salt thereof, obtained by precipitation and filtration is further purified by column chromatography.

At Step S-2 (amide formation), fragment F-2, or a salt thereof, and fragment F-3, or a salt thereof, participate in an amide forming reaction to form a compound of formula I, or a salt thereof. Suitable amide forming reactions are well known to one of ordinary skill in the art and typically involve an activated ester moiety such that treatment with a amine moiety results in the formation of an amide bond. The coupling reaction is typically carried out in the presence of an excess of a base. In some embodiments the base is a tertiary amine base. In some embodiments, the tertiary amine base is triethylamine. In some embodiments the base is a tertiary amine base. In some embodiments, the tertiary amine base is DIPEA. The coupling reaction may be carried out in a suitable solvent that solubilizes all of the reagents. In some embodiments, the solvent is a dipolar aprotic solvent. In some embodiments, the dipolar aprotic solvent is DMA. In some embodiments, the dipolar aprotic solvent is DMSO, DMF, acetone, ethyl acetate, HMPA or DMPU. In some embodiments, the reaction mixture is mixed with a non-polar solvent to precipitate out the compound of formula I, or a salt thereof. In some embodiments, the reaction mixture is mixed with a non-polar solvent at room temperature or a lower temperature to form a suspension or slurry. In some embodiments, the suspension or slurry is further stored at room temperature or a lower temperature for a period of time, with or without mixing, before a compound of formula I, or a salt thereof, is filtered out. In some embodiments, a lower temperature is about 15° C., 10° C., 5° C., 0° C., −5° C., −10° C., −15° C., or −20° C. In some embodiments, a lower temperature is below −20° C. In some embodiments, a non-polar solvent is an ether. In some embodiments, a non-polar solvent is diethyl ether. In some embodiments, a non-polar solvent is methyl tert-butyl ether (MTBE). In some embodiments, a compound of formula I, or a salt thereof, obtained by precipitation and filtration is of a purity of about 70% or higher. In some embodiments, a compound of formula I, or a salt thereof, obtained by precipitation and filtration is of a purity of about 72%, 74%, 76%, 78%, 80%, 82%, 84%, 86%, 88%, 90%, 92%, 94%, 96%, or 98%. In some embodiments, a compound of formula I, or a salt thereof, obtained by precipitation and filtration is further purified by column chromatography.

In some embodiments, the present invention provides a method for preparing fragment F-2, or a salt thereof, comprising steps of 1) providing fragment F-1, or a salt thereof; 2) reacting fragment F-1, or a salt thereof, with compound A, or a salt thereof, to form fragment F-2, or a salt thereof; and 3) separating fragment F-2, or a salt thereof, from reaction mixture by precipitation, wherein each of compound A and fragments F-1 and F-2 is as described above. In some embodiments, the method further comprises purifying fragment F-2, or a salt thereof, by column chromatography. In some embodiments, solvents and conditions of the method are as described for step S-1 above.

In some embodiments, the present invention provides a method for preparing a compound of formula I, or a salt thereof, comprising steps of 1) providing fragment F-2, or a salt thereof; 2) reacting fragment F-2, or a salt thereof, with fragment F-3, or a salt thereof, to form a compound of formula I, or a salt thereof; and 3) separating the compound of formula I, or a salt thereof, from reaction mixture by precipitation, wherein each of fragment F-2 and F-3, and a compound of formula I is as described above. In some embodiments, the method further comprises purifying the compound of formula I, or a salt thereof, by column chromatography. In some embodiments, solvents and conditions of the method are as described for step S-2 above.

In some embodiments, the present invention provides a method for preparing a compound of formula I, or a salt thereof, comprising steps of 1) providing fragment F-1, or a salt thereof; 2) reacting fragment F-1, or a salt thereof, with compound A, or a salt thereof, to form fragment F-2, or a salt thereof; 3) separating fragment F-2, or a salt thereof, from reaction mixture by precipitation; 4) reacting fragment F-2, or a salt thereof, with fragment F-3, or a salt thereof, to form a compound of formula I, or a salt thereof; and 5) separating the compound of formula I, or a salt thereof, from reaction mixture by precipitation. In some embodiments, the method further comprises purifying the compound of formula I, or a salt thereof, by column chromatography. In some embodiments, fragment F-2, or a salt thereof, obtained from step 3) is not further purified by column chromatography before being used in step 4). In some embodiments, solvents and conditions of the method are as described for steps S-1 and S-2 above.

In some embodiments, the present invention provides a heterogeneous mixture comprising fragment F-2, or a salt thereof, and a non-polar solvent. In some embodiments, a heterogeneous mixture is a suspension. In some embodiments, a heterogeneous mixture is a slurry. In some embodiments, the present invention provides a solid composition comprising fragment F-2, or a salt thereof, and a small amount of a non-polar solvent. In some embodiments, the heterogeneous mixture and/or solid composition further comprise TBTU. In some embodiments, the non-polar solvent in the heterogeneous mixture and/or solid composition is as described for step S-1 above. In some embodiments, the temperature of the heterogeneous mixture and/or solid composition is as described for step S-1 above. In some embodiments, purity of fragment F-2, or a salt thereof, after being filtered out of the heterogeneous mixture is as described for step S-1 above. In some embodiments, purity of fragment F-2, or a salt thereof, in the solid composition is as described for step S-1 above.

In some embodiments, the present invention provides a heterogeneous mixture comprising a compound of formula I, or a salt thereof, and a non-polar solvent. In some embodiments, a heterogeneous mixture is a suspension. In some embodiments, a heterogeneous mixture is a slurry. In some embodiments, the present invention provides a solid composition comprising a compound of formula I, or a salt thereof, and a small amount of a non-polar solvent. In some embodiments, the non-polar solvent in the heterogeneous mixture and/or solid composition is as described for step S-2 above. In some embodiments, the temperature of the heterogeneous mixture and/or solid composition is as described for step S-2 above. In some embodiments, purity of compound of formula I, or a salt thereof, after being filtered out of the heterogeneous mixture is as described for step S-2 above. In some embodiments, purity of compound of formula I, or a salt thereof, in the solid composition is as described for step S-2 above.

### 4. Description of Exemplary Bicycle Toxin Conjugates

In some embodiments, a Bicycle toxin conjugate of formula I is:

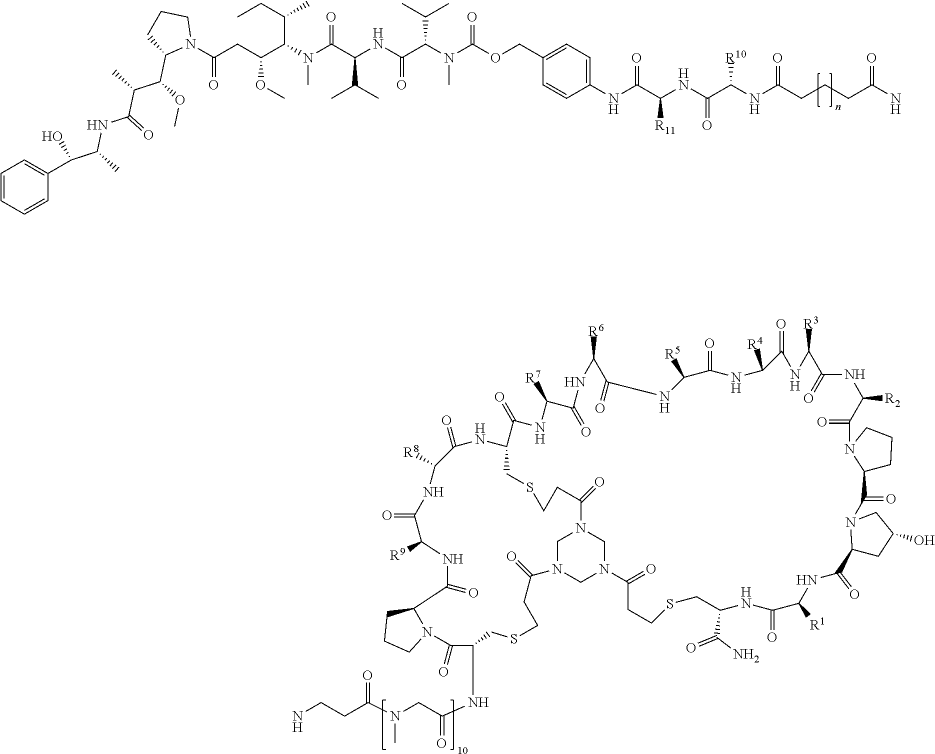

or a pharmaceutically acceptable salt thereof, wherein each of R¹, R², R³, R⁴, R⁵, R⁶, R⁷, R⁸, R⁹, R¹⁰, R¹¹, m, and n is as defined below and described in embodiments herein, both singly and in combination.

In some embodiments, a Bicycle toxin conjugate of formula I is:

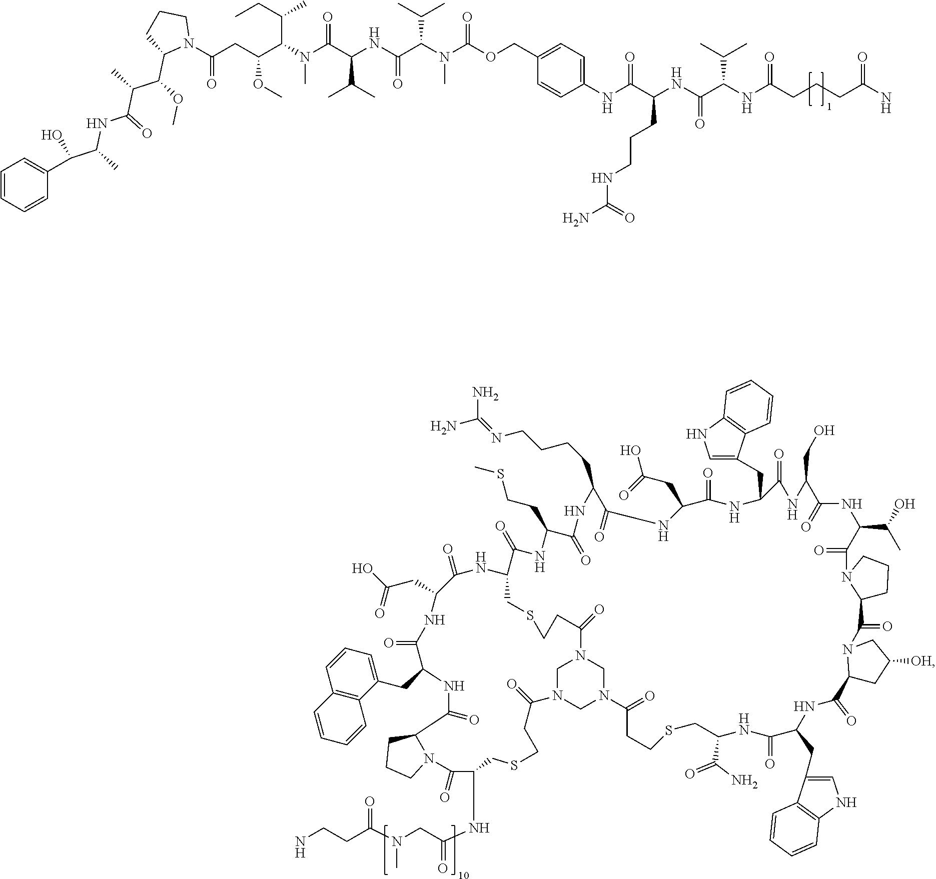

or a pharmaceutically acceptable salt thereof.

In some embodiments, a Bicycle toxin conjugate of formula I is BT8009, or a pharmaceutically acceptable salt thereof.

### 5. Uses, Formulation and Administration

### Pharmaceutically Acceptable Compositions

According to another embodiment, the invention provides a composition comprising a Bicycle toxin conjugate of this invention, or a pharmaceutically acceptable derivative thereof, and a pharmaceutically acceptable carrier, adjuvant, or vehicle.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution.

These solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some embodiments, suitable formulations for lyophilization and reconstitution for use in parenteral administration by dilution into an infusion solution containing, for example, isotonic saline or dextrose, may comprise one or more of the following excipients:
an acid buffering component such as citric acid, succinic acid or acetic acid, or an amino acid such as glycine or histidine;
a base such as sodium hydroxide or potassium hydroxide or an organic base such as tris(hydroxymethyl)aminomethane;
a mineral acid such as HCl to adjust the pH within the desired range typically pH 3-9;
a dispersant or surfactant, such as polysorbate 20 or polysorbate 80; and/or
a sugar to provide lyophilized product stability and to control water content, for example sucrose, lactose, dextrose, trehalose or mannitol.
   The mixture is typically lyophilized from aqueous solution and reconstituted in purified water prior to dilution into the desired infusion solution.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable gel, ointment, lotion, or cream containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

In some embodiments, the present invention provides a method for preventing and/or treating cancers as described herein comprising administering to a patient a Bicycle toxin conjugate of the invention.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, a cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, a cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, a cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, a patient is an adult human. In some embodiments, a patient is a child or pediatric patient.

In some embodiments, a cancer includes, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, a cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, a cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, a cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is hepatocellular carcinoma (HCC). In some embodiments, a cancer is hepatoblastoma. In some embodiments, a cancer is colon cancer. In some embodiments, a cancer is rectal cancer. In some embodiments, a cancer is ovarian cancer, or ovarian carcinoma. In some embodiments, a cancer is ovarian epithelial cancer. In some embodiments, a cancer is fallopian tube cancer. In some embodiments, a cancer is papillary serous cystadenocarcinoma. In some embodiments, a cancer is uterine papillary serous carcinoma (UPSC). In some embodiments, a cancer is hepatocholangiocarcinoma. In some embodiments, a cancer is soft tissue and bone synovial sarcoma. In some embodiments, a cancer is rhabdomyosarcoma. In some embodiments, a cancer is osteosarcoma. In some embodiments, a cancer is anaplastic thyroid cancer. In some embodiments, a cancer is adrenocortical carcinoma. In some embodiments, a cancer is pancreatic cancer, or pancreatic ductal carcinoma. In some embodiments, a cancer is pancreatic adenocarcinoma. In some embodiments, the cancer is glioma. In some embodiments, a cancer is malignant peripheral nerve sheath tumors (MPNST). In some embodiments, a cancer is neurofibromatosis-1 associated MPNST. In some embodiments, a cancer is Waldenstrom's macroglobulinemia. In some embodiments, a cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

In some embodiments, a cancer is treated by arresting further growth of the tumor. In some embodiments, a cancer is treated by reducing the size (e.g., volume or mass) of the tumor by at least 5%, 10%, 25%, 50%, 75%, 90% or 99% relative to the size of the tumor prior to treatment. In some embodiments, a cancer is treated by reducing the quantity of the tumor in the patient by at least 5%, 10%, 25%, 50%, 75%, 90% or 99% relative to the quantity of the tumor prior to treatment.

The compounds and compositions, according to the method of the present invention, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered parenterally at dosage levels of about 0.01 mg/kg to about 50 mg/kg and preferably from about 1 mg/kg to about 25 mg/kg, of subject body weight per day.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration of a compound of this invention include ointments, pastes, creams, lotions, gels, foams, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### List of Common Abbreviations Used in the Experimental Section.

AA(s): Amino acid(s)
ACN: Acetonitrile
Ac₂O: Acetic anhydride
AcOH: Acetic acid
API: Active Pharmaceutical Ingredient
Aq.: Aqueous
A %: Peak Area Percent
1,4-BDMT: 1,4-Benzenedimethanethiol
Boc: t-Butyloxycarbonyl
BV: Bed volume
° C.: Degrees Celsius
C of A: Certificate of Analysis
Cat: Category
CPP: Current Preferred Procedure
CV: Column Volumes
DIC: Diisopropylcarbodiimide
DIPEA: Diisopropylethylamine
DITU: Diisopropylthiourea
DMA: N,N-Dimethylacetamide
DMF: Dimethylformamide
DM1: Mertansine/Emtansine
DTT: 1,4-Dithiothreitol
eq.: Molar Equivalents
Eq.: Equivalent
Expt: Experiment
h: Hours
H: Hour
HPLC: High Performance Liquid Chromatography
Imp: Impurity
Info: Information
IPA: Isopropyl alcohol
IPC: In-process control
Lab: Laboratory
LC: Liquid Chromatography
Lyo: Lyophilization
MBHA: 4-Methylbenzhydrylamine
Fmoc: Fluorenylmethyloxycarbonyl
MeOH: Methanol
Min: Minute
mL: Milliliter
Mol: Moles
Mol. Wt.: Molecular Weight
MTBE: Methyl tert-butyl ether
non-GMP: Non-Good Manufacturing Practices
NMT: No more than
Oxyma: Ethyl cyano(hydroxyimino)acetate
PD: Process development
Pdt: Product
RO/DI: Reverse Osmosis
RP-HPLC: Reversed-phase high performance liquid chromatography
RP-18: Reverse Phase C18-bonded Silica
RRT: Relative Retention Time
R^{†}: Room temperature
SAFC: Sigma-Aldrich Fine Chemicals
SM: Starting material
SPP: N-Succinimidyl 2-pyridyldithio-carboxylate
SPPS: Solid-phase peptide synthesis
TATA: 1,3,5-Triacryloylhexahydro-1,3,5-triazine
TFA: Trifluoroacetic Acid
TIPS: Triisopropylsilane
TLC: Thin layer chromatography
USP: United States Pharmacopeia
v/v: Volume/Volume
Vol: Volume
wt %: Weight Percent
Wt: Weight

### Example 1: Preparation of Bicycle BCY8234

The synthesis of BCY8234 was revisited with the aim to reduce the high level of aspartimide related impurities found previously. A series of experiments were performed with different deblocking cocktails, and the cocktail with 3% oxyma in 10% piperidine/DMF was selected for the process moving forward.

The cleavage from the resin, and global deprotection of the peptide were performed in a single step, using TFA cocktails comprising of 90% TFA, 15% DTT, and 5% TIPS, 0.25% NH₄I and 5% water. 150 g of peptide-resin was cleaved to produce 142 g of crude linear peptide with spent resin, after precipitation and drying.

Cyclization of the linear peptide was performed with TATA in basic conditions to produce the crude cyclic product. Two sets of cyclization experiments with total of 121 g of crude linear peptide with spent resin were performed, each with linear crude from different cleavage methods. The quality of the cyclic crude solutions was similar.

Initial purification was accomplished by Reverse-Phase HPLC using C18 column media (Daiso Gel, 120 Å, 10) with 0.1 M NH₄OAc in Water/ACN buffer system. This was then followed by 0.1% TFA in water/ACN purification in the same reverse phase C18 column. The TFA main pool was desalted with water/ACN and lyophilized to obtain approximately 24 g of final product with purity>95%.

Sequence is β-Ala¹-Sar²-Sar³-Sar⁴-Sar⁵-Sar⁶-Sar⁷-Sar⁸-Sar⁹-Sar¹⁰-Sar¹¹-*Cys¹²-Pro¹³-1Nal¹⁴-d-Asp¹⁵-*Cys¹⁶-Met¹⁷-hArg¹⁸-Asp¹⁹-Trp²⁰-Ser²¹-Thr²²-Pro²³-Hyp²⁴-Trp²⁵-*Cys-NH2

Wherein the * denotes a cysteine residue that forms a bicyclic thioether with 1, 1, 1″-(1, 3, 5-triazinane-1, 3, 5-triyl)tris(propan-1-one) as below.

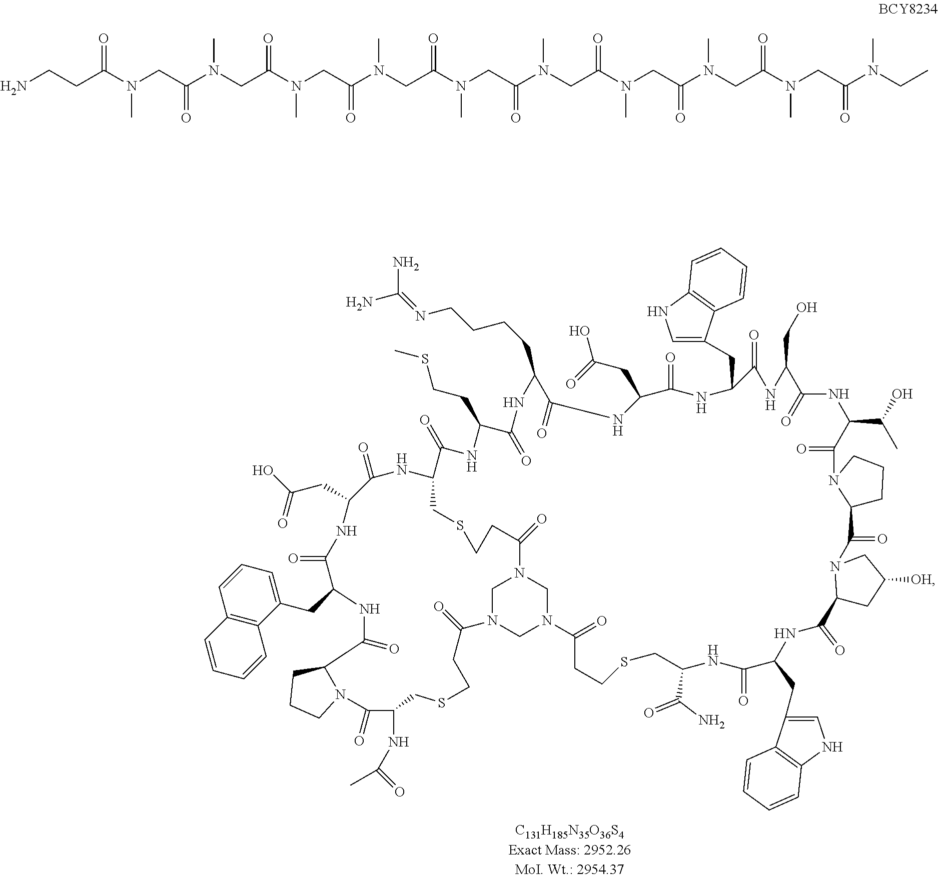 [SMILES: CCN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCN ; CSCC[C@@H]1NC(=O)[C@@H]2CSCCC(=O)N3CN(CN(C3)C(=O)CCSC[C@H](NC(C)=O)C(=O)N3CCC[C@H]3C(=O)N[C@@H](Cc3cccc4ccccc34)C(=O)N[C@H](CC(=O)O)C(=O)N2)C(=O)CCSC[C@@H](C(N)=O)NC(=O)[C@H](Cc2c[nH]c3ccccc23)NC(=O)[C@@H]2C[C@@H](O)CN2C(=O)[C@@H]2CCCN2C(=O)[C@H]([C@@H](C)O)NC(=O)[C@H](CO)NC(=O)[C@@H](Cc2c[nH]c3ccccc23)NC(=O)[C@H](CC(=O)O)NC(=O)[C@H](CCCCN=C(N)N)NC1=O]

### Solid Phase Synthesis of BCY8234

The synthesis of BCY8234 protocol used for the last GMP batch needed to be optimized because of the high presence of aspartimide related impurities. The aspartimide impurities are believed to be formed during the deblocking process with 20% piperidine in DMF. The amount of aspartimide impurities were reduced from 20.4% to 18.6% by adding 0.1 M oxyma to the deblocking solution.

Earlier attempts used 0.15 M oxyma in 3% piperidine/DMF to suppress these impurities. The drawback to this cocktail was the presence of incomplete deblocking of Fmoc protection group confirmed by the deleted sequence impurities present in the crude linear peptide.

Based on the data from earlier work and previous knowledge, optimization experiments were designed aimed at mitigating the aspartimide problem.

In the addition to the aspartimide reduction, new improvement techniques were tested. These techniques include 60-minute pre-activation, oxidation suppression with DITU and one post coupling wash to decrease the amount of DMF. Furthermore, the experiments were performed on a fully loaded resin (substitution>0.8 mmoles/g) to see if there is any advantage to partial loading used in the previous work.

### Synthesis Optimization

The synthesis optimization experiments were performed on the Symphony XTM Synthesizer. Three factors were screened: the concentrations of piperidine, oxyma, and formic acid in the deblocking cocktail. Minitab was used to design the screening experiments. The first four experiments were designed to look for trends and relationships. The experiments are described below:
Deblocking reactions are 5′ and 20′
2 eq. of amino acids, 2 eq. of oxyma, and 2.1 eq. of DIC
Fmoc-Sar-Sar-OH was used in place of Fmoc-Sar-OH
60-minute activation times (except Cys and homoArg residues)
0.2 eq. of DITU was added to every coupling solution.
Coupling time was 3 hours.
No acetylation
1 post coupling washes

The results of the experiments are summarized in Table 1.

TABLE 1
Deblocking Cocktail Screening Experiments

| | | | Crude | SPPS | |
|---|---|---|---|---|---|
| Experiment | Piperidine | 3% | purity, | yield, | aspartimide, |
| No. | % | Additive | % | % | % |
| 1 | 5 | oxyma | 68.3 | 104.4 | 5.9 |
| 2 | 10 | oxyma | 69.2 | 105 | 5.7 |
| 3 | 5 | formic | 68.9 | 45.6 | 5.8 |
| | | acid | | | |
| 4 | 10 | formic | 62.5 | 95.75 | 5.4 |
| | | acid | | | |

### Discussion

The results ofthese experiments confirmed that addition of organic acids can reduce the aspartimide formation. Ofthe two acids tested, the less reactive oxyma shows a positive impact on the synthesis yield. The 300 formic acid in 500 piperidine/DWVI (Run No. 3) lowered the synthesis yield to below 50% as observed by analytical HPLC analysis of the crude sample. Oxyma was further investigated in the next set of experiments.

### Additional Oxyma and Piperidine Concentration Experiments

In this section, the effect of increasing the oxyma concentration to 500 in the piperidine solution was investigated. The effect of removing the final deblocking by using Boc-β-Ala-OH in the final coupling was tested in experiment #6, in comparison with Fmoc-β-Ala-OH #5. An additional deblocking condition suggested by BicycleTX was also evaluated in experiment #7. The results are shown in Table 2.

TABLE 2
Deblocking Experiments Continued

| | | | Reaction | | SPPS | |
|---|---|---|---|---|---|---|
| Experiment | Piperidine | | Time | Crude | yield, | Aspartimide, |
| No. | (%) | Additive | (min) | purity, % | (%) | (%) |
| 5 | 10 | 5% oxyma | 5 and 20 | 67.3 | 104 | 10.3 |
| 6 | 10 | 5% oxyma | 5 and 20 | 67.2 | 103 | 6.3 |
| 7 | 3 | 0.15M oxyma | 3 × 10 mins | 38.7 | 90.7 | 5.6 |

The initial deblocking condition gave the worst crude purity and lower yield. There is no significant difference in yield and purity when the Fmoc-β-Ala-OH was substituted with Boc-β-Ala-OH. The aspartimide related impurities are unusually high for experiment #5 and appear to be out of trend when compared to the rest of conditions.

The quality of the crude peptide from synthesis can be improved by employing methods that limits aspartimide formation without causing deletions or truncations of the sequence. The use of formic acid caused truncation which can be attributed to formylation of the free amine. This is prevalent in Exp. Std order #3 with 45.6% yield. In addition, due to the acidity of formic acid (pKa=3.75), it may have caused some of the identified deletion sequences or Des impurities, by reducing the effectiveness of the piperidine solution. Because of these reasons, formic acid was deemed a poor addictive for prevention of aspartimide formation.

The use of oxyma (pKa=4.60) to buffer the basicity of the piperidine solution worked better than formic acid. This may be because oxyma is less reactive and does not cause any truncations of the sequence. While the designed experiments gave similar results for use of oxyma addictive, the replication of the initial protocol was inferior. The condition employed in experiment std. order #2 was selected for the GMP manufacturing process.

### Cleavage and Global Deprotection

### Cleavage Optimization Experiments:

A series of cleavage experiments were performed to find the best conditions for cleaving the peptide from the resin. First the various TFA cocktails were tested. Then, cocktail to resin ratio was evaluated to find the best reaction concentration for the cleavage. After cocktail and reaction concentration, the operational temperature was tested. The cleavage reactions were performed with 10 g of peptide-resin for 3 hours and −40° C. MTBE (4×) was used to precipitate the peptide with the spent resin.

### TFA Cocktail Selection Experiments

### Comparison of 1,4-BDMT to DTT as Thiol Scavenger

1,4-Benzenedimethanethiol (1,4-BDMT) has been reported by PPL to be a superior scavenger to DTT (Pawlas and Rasmussen, Green Chemistry 2019 (21) 5990-5998). This reagent was tested in the cleavage optimization. The experiments performed are summarized below.
10 mL/g cleavage concentration
Cocktail: 85% TFA, 5% water 5% TIPS, 0.2% NH₄I and 5% DTT or 1,4-BDMT
Cocktail cooled to 10±2° C.
TIPS added after 1 h
3 h reaction time at RT

The result is summarized in Table 3.

TABLE 3
DTT vs. 1,4-BDMT Experiment

| Thiol | Purity (%) | +56 (%) | +163 (%) |
|---|---|---|---|
| DTT | 58.93 | 19.82 | 4.51 |
| BDMT | 61.39 | 16.34 | 5.34 |

While the 1,4-BDMT cleavage reduced the t-butylation by 3.5%, the overall purity is quite similar albeit slightly improved with 1,4-BDMT by 2.5%. Since the difference is not significant enough to introduce a new chemical to the process, DTT was used for further optimization of the cleavage process.

### Cocktail Screening Experiments

Minilab was used to design the experiments
10 mL/g cleavage concentration
Solid scavengers (DTT & NIH4) are excluded in total cocktail volume
NH₄I remains 0.2% of Total Volume
Amount of water in cocktail=amount of TIPS
Total volume=TFA+water+TIPS
Cocktail cooled to 10±2° C.
TIPS added after 1 h
3 h reaction time at RT

TABLE 4
TFA Cocktail Screening

| Std | Run | Center | | TFA | DTT | Purity | +56 | +163 |
|---|---|---|---|---|---|---|---|---|
| Order | Order | Pt | Blocks | (%) | (%) | (%) | (%) | (%) |
| 1 | 1 | 1 | 1 | 90 | 5 | 60 | 19.46 | 4.14 |
| 4 | 2 | 1 | 1 | 95 | 10 | 65.53 | 16.93 | 4.46 |
| 5 | 3 | 0 | 1 | 92.5 | 7.5 | 62.02 | 17.23 | 4.24 |
| 2 | 4 | 1 | 1 | 95 | 5 | 58.86 | 22.42 | 4.55 |
| 3 | 5 | 1 | 1 | 90 | 10 | 66.09 | 15.67 | 4.9 |

### Data Analysis

The results of the cocktail screening experiments were analyzed using Minitab. The analyses are described below.

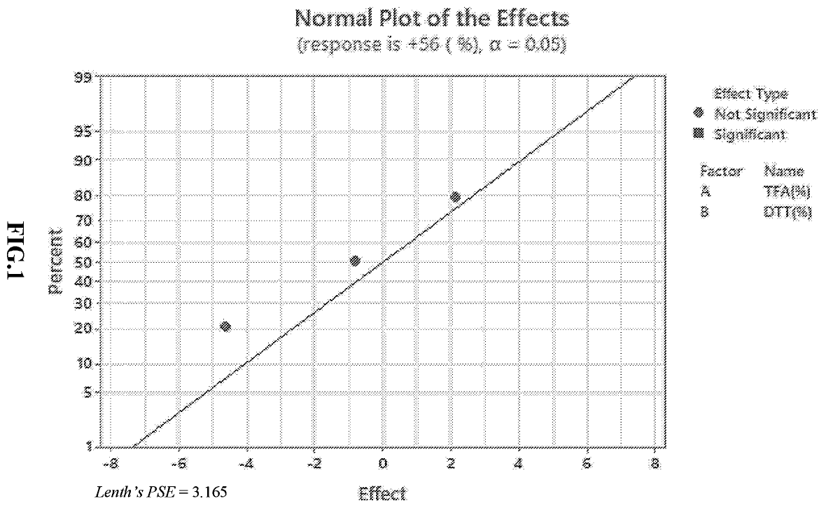

Factorial Regression: +56(%) versus TFA (%), DTT (%), CenterPt is shown in FIG. 1 for the +56 impurity.

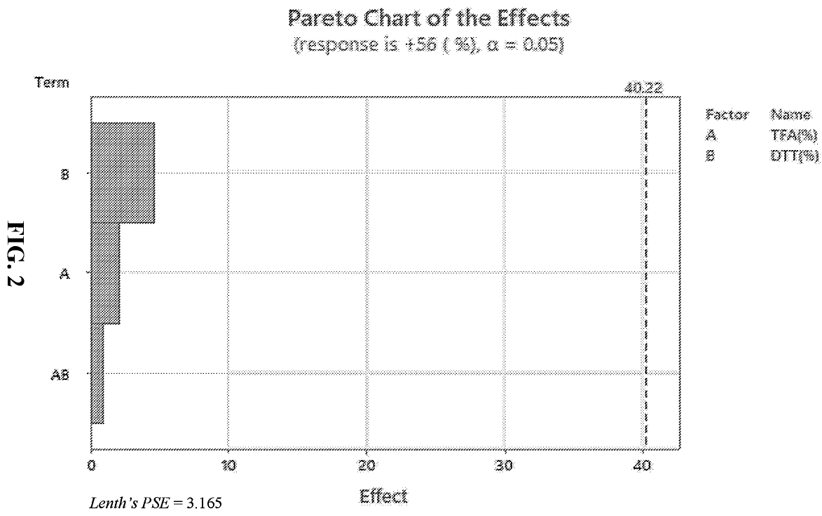

The Pareto chart of the effects for the +56 impurity is shown in FIG. 2. Note that it was not possible to graph the specified residual type because MSE=0 or the degrees of freedom for error=0.

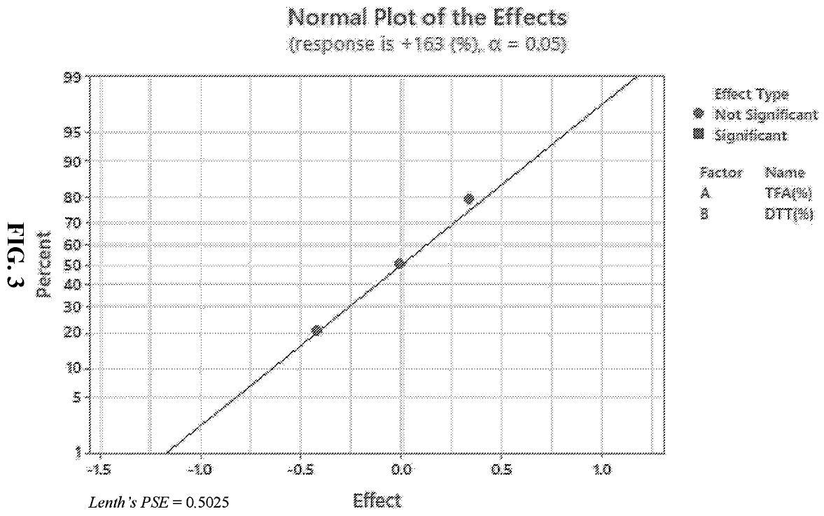

Factorial Regression: +163(%) versus TFA (%), DTT (%), CenterPt is shown in FIG. 3 which depicts the normal plot of the effects for the +163 impurity.

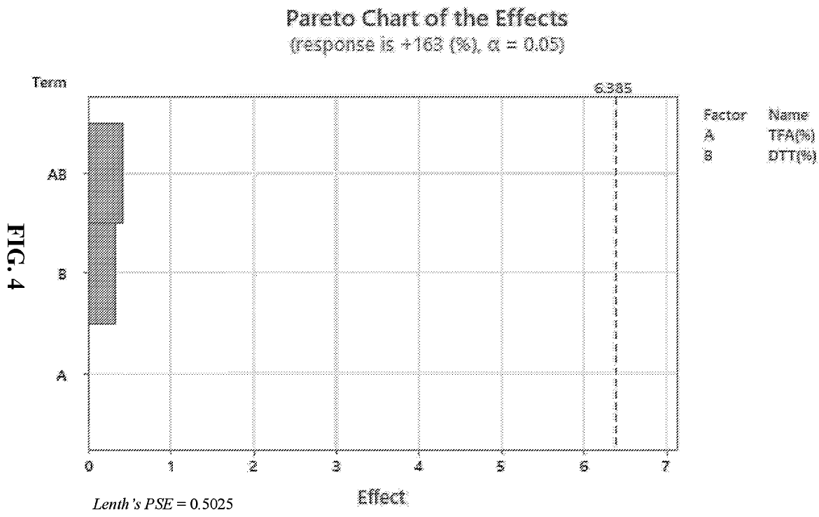

The Pareto chart of the effects for the +163 impurity is shown in FIG. 4. Note that it was not possible to graph the specified residual type because MSE=0 or the degrees of freedom for error=0.

Response Optimization: +163(%), +56(%), Purity (%) is shown in Table 5 below.

TABLE 5
Response Optimization

| | TFA | DTT | +163 (%) | +56 (%) | Purity (%) | Composite |
|---|---|---|---|---|---|---|
| Solution | (%) | (%) | Fit | Fit | Fit | Desirability |
| 1 | 95 | 10 | 4.46 | 16.93 | 65.53 | 0.757353 |

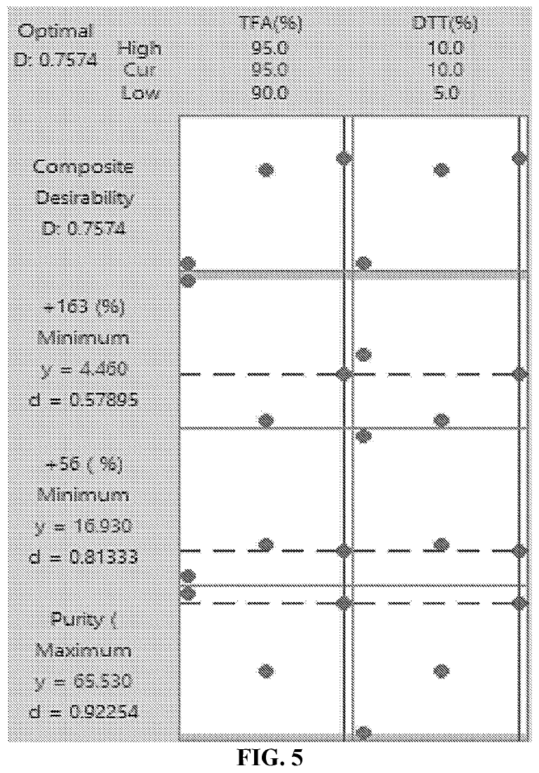

FIG. 5 depicts the response optimization for cleavage cocktails.

The TFA and DTT content show no significant effect on both impurities targeted. While the Minitab response optimizer selected standard order #4 the team selected standard order #3 as the better result. The 10% DTT cocktails were better than 5% DTT. So, the liquid mixture (i.e., TFA, water & TIPS) was tested in the 15% DTT experiments.

### % DTT Experiments

The initial screening experiments show that increasing DTT amount 10% improved the crude quality. The two cocktails with 10% DTT will be increased to 15% DTT and compared to the current BPR cocktail.
15% DTT used for all experiments
Solid scavengers (DTT & NH₄I) are excluded in total cocktail volume
NH₄I remains 0.25% of Total Volume
Amount of water in cocktail=amount of TIPS
Total volume=TFA+water+TIPS
Cocktail cooled to 10±2° C.
TIPS added after 1 h
3 h reaction time at RT
The experiments are summarized in Table 6 below

TABLE 6
15% DTT experiments

| | TFA | | Temp | Purity | +56 | +163 |
|---|---|---|---|---|---|---|
| Exp | (%) | mL/g | (° C.) | (%) | (%) | (%) |
| 6(BPR) | 92.5 (5% TIPS) | 10 | 22 | 69.90 | 14.74 | 6.99 |
| 7 | 90 | 10 | 23 | 70.67 | 13.29 | 6.27 |
| 8 | 95 | 10 | 21 | 69.04 | 15.05 | 6.58 |

There is no significant difference among the three experiments, but some improvement in terms of over-all purity is observed from the 10% DTT results (Table 4). Experiment 7 was slightly better and was selected for concentration and temperature experiments.

### Cocktail to Resin Ratio (Cleavage Concentration) Experiment

After selection of the cocktail composition, the cleavage concentration, i.e., the ratio of cocktail to resin (mL/g) was evaluated. The experiments were performed using cocktail #7 (Table 6) with 10 g peptide-resin each. The result of this experiment is reported in Table 7 below.

TABLE 7
Cocktail to Resin Ratio

| | TFA | | Temp | Purity | +56 | +163 |
|---|---|---|---|---|---|---|
| Exp | (%) | mL/g | (C.) | (%) | (%) | (%) |
| 9 | 90 | 12 | 22 | 72.91 | 14.33 | 6.28 |
| 10 | 90 | 15 | 23 | 71.90 | 13.52 | 6.55 |

Since there is no improvement on the purity or crude recovery with any of the ratios tested, the 10 mL/g ratio was maintained for the large-scale cleavage.

### Cleavage Temperature Experiments

With the cocktail and concentration selection, the next step will be to check if the temperature plays significant influence on the purity or yield. Cooler temperature. (15° C.) and a warmer temperature (30° C.) were tested, and the results of these experiments are reported below.

TABLE 8
Cleavage Temperature results

| | TFA | | Temp | Purity | +56 | +163 |
|---|---|---|---|---|---|---|
| Exp | (%) | mL/g | (C.) | (%) | (%) | (%) |
| 11 | 90 | 10 | 15 | 73.01 | 13.11 | 6.41 |
| 12 | 90 | 10 | 30 | 67.70 | 13.06 | 10.76 |

While the results for exp. 11 (15° C.) the best crude purity, the recovery from this cleavage was very low; 67% less than the other cleavages. The 30° C. cleavage has lower crude purity. Therefore, the cleavage conditions deemed optima was to remain at room temperature.

### Summary and Conclusion of Cleavage Conditions

The obvious trend observed in the optimization experiments is that purity of the crude improves as the DTT amount increases from 5% to 15%. The optimal condition for the cleavage is a cocktail of 90% TFA, 5% water, 15% DTT, 0.25% ammonium iodide and 5% TIPS (added after 1 h). The 90% TFA+5% water+5% TIPS=100% (10 mL/g). The 15% DTT and 0.25% NH₄I are added on top. Cocktail cooled to 10±2° C. before resin addition. The total reaction time is 3 h at room temperature. The crude with spent resin is precipitated with 4 times the cocktail volume of cold MTBE (≤−30° C.) and the precipitate washed 3 times with MTBE.

Determination of Crude Yield without Spent Resin

Two cleavages of 10 g each using the optimized conditions described above. In one cleavage the Crude was isolated without resin, and the other was with spent resin (control). The isolated spent resin is washed with methanol and dried for weight determination. The results of the experiments are summarized below.

TABLE 9
Results Summary

| Experiment # | With Resin? | Yield (g) | Spent resin (g) | Purity (%) |
|---|---|---|---|---|
| 1 | Yes | 9.6 | n/a | 71.05 |
| (control) | | | | |
| 2 | No | 6.4 | 3.2 | 71.18 |

Based on the above results, it can be estimated that there is a 67% of crude in the crude+spent resin isolated from cleavage.

### Large Scale Cleavage Demonstration

A 150 g cleavage was performed to test the scalability of the optimized cleavage conditions. The cleavage and result are summarized below.
150 g peptide resin was used
Cocktail: 90% TFA (1350 mL), 5% (75 mL) water, 15% (225 g) DTT, 0.25% (3.75 g) NH₄I, and 5% (75 mL) TIPS
Cocktail cooled to 10±2° C.
TIPS added after 1 h
3 h reaction time at RT
Precipitated with 4 times −40° C. MTBE (6 L), and washed 3 times with 150 mL MTBE
Recovered 142 g after drying with Purity=74.04%.

### Cyclization

### Cyclization Optimization Experiments

To find the best procedure for the cyclization reaction, a series of experiments were performed. Two different setups were investigated; the current method in the BPR, and the setup provided by Bicycle. 2.5 g of crude linear peptide (purity˜71.6%) was used to perform four experiments shown in Table 10. Concentration of reactants and addition times were tested. The current PPL protocol (3 pots) used 2 eq. of TATA while the Bicycle method (2 pots) used 1.3 eq. The reactions were quenched after 24 hours by addition of 6.5 eq. (43 mg) Ac-Cys-OH and stirred for 1 hour. The pH of the solutions was then adjusted to pH=4 with acetic acid.

TABLE 10
2.5 g Scale Cyclization Experiments

| | Linear | TATA | Addition | ACN | Conc. | |
|---|---|---|---|---|---|---|
| Exp | eq. | (eq) | Time | (%) | (g/L) | Purity |
| 1(BPR) | 1 | 2 | 2 h | 30 | 5 | 63.25 |
| 2 | 1 | 1.3 | 1 h | 50 | 6.6 | 53.42 |
| 3 | 1 | 2 | 2 h | 30 | 7.5 | 53.54 |
| 4 | 1 | 1.3 | 1 h | 50 | 10 | 63.04 |

### Discussion

The results show no trend in purity, when a final crude concentration was increased from 5 to 10 g/L. The use of 50% ACN is not beneficial since 3× dilution before loading the crude peptide onto the column is required. TATA equivalent can be reduced to 1.3 eq. without any purity drop. The experiment and results are described in Table 10.

### Comparison of 2 Pot to 3 Pot Set Up

The original 3-pot set up was compared to the 2-pot setup suggested by Bicycle. Both reactions were performed with 5 g/L in 30% ACN/0.1 M NH₄HCO₃. 1.3 eq. of TATA added over 2 hours, and the reaction quenched after 24 hours.

TABLE 11
3 pot vs. 2 pot

| Exp. | Pot 1 (g/L) | Pot 2 (g/L) | Pot 3 (reactor) | Purity (%) |
|---|---|---|---|---|
| 5 | 20 (linear) | 10 (TATA) | 5 g/L | 70.05 |
| 6 | 20 (linear) & 2 | n/a | 5 g/L | 69.98 |
| | (TATA) | | | |

### Discussion

Both methods give similar results. While the 2-pot setup appears to be attractive because of less equipment required, the 3-pot setup has been standardized for TATA safety and will remain the setup for GMP manufacturing.

### Cyclization Experiment (Reduction of Acetonitrile %)

Using 30% acetonitrile in water for cyclization means that the solution must be diluted two times with water before loading onto the purification column. This means higher volume and longer loading time. Therefore, to bypass this problem, the concentration of acetonitrile in the cyclization solution needed to be reduced. The following experiments were performed to see if acetonitrile content could be reduced during cyclization. 3.7 g of linear crude+spent resin (purity˜70.86%). The results are summarized in Table 12 below.

TABLE 12
Acetonitrile Content test for Cyclization

| | ACN Concentration | Cyclic Crude |
|---|---|---|
| Experiment # | (%) | Purity (%) |
| 7 | 30 | 69.95 |
| 8 | 25 | 71.71 |
| 9 | 20 | 72.8 |
| 10 | 15 | 70.9 |

### Discussion

There appear to be a slight increase in purity as the acetonitrile percent is reduced. No

cloudiness or precipitation was observed at 20% ACN, some precipitation was observed at 15% ACN. Therefore, 20% ACN final solution will be used for large-scale cyclization.

### Large Scale Cyclization

The large-scale cyclization was performed in a 22 L 3-neck flask with constant nitrogen bubble. Two reactions were performed with 121 g linear with spent resin. The first reaction was performed with the linear+spent resin from the 150 g cleavage, while the second cyclization was performed with the combined remaining crude+spent resin from cleavage optimization process. The protocol for the cyclization reaction is described below.

### Procedure

Prepared 0.15 M solution of NH₄HCO₃ (130 g, 1.65 moles) in 10.75 L of 16.3% ACN/water (1.75 L ACN & 9 L H₂O), under N₂.
121 g (27.7 mmoles) of linear peptide was dissolved in 20% ACN/water (3.75 L), under N₂.
Filter the linear solution and wash the spent resin 3×250 mL of 20% ACN/water.
9.6 g (38.6 mmoles) TATA was dissolved in 1 L of 50% ACN
TATA and crude linear peptide were added over 2 hours to the stirred NH₄HCO₃ solution under nitrogen.
The final reaction volume was 16.25 L
Reaction was monitored with HPLC for completion.
Dissolved 41 g of Ac-Cys-OH (250 mmoles) in 500 mL of water
When the reaction was complete, the Ac-Cys-OH solution was added to the reaction flask to quench the reaction
Stirred the reaction for 1 more hour
Added 800 mL of 50% AcOH in water to the reaction mixture to adjust the pH to 4.5. for the first sublot. The pH was adjusted to pH=6.8 for the second sublot.
Proceeded to purification.

TABLE 13
Large Scale Cyclization

| | Linear Crude | Ext. Product | Cyclic Crude |
|---|---|---|---|
| Cyclization # | Purity (%) | content (g) | Purity (%) |
| 1 | 70.67 | 18 | 74.32 |
| 2 | 68.78 | 18 | 72.48 |

### Purification

### Purification Optimization

The current purification method was tested first to see if it is good enough to purify the higher quality crude product from the optimized upstream processes. The loading amount is tested for each stage. RPC3 is added for TFA desalting.

### RPC 1 Purification Test

The combined crude from cyclization was purified using the existing purification method described below:
Column media: Daiso Gel C18 120 Å, 10 m
Buffer A=0.1 M NH₄OAc, buffer B=ACN
Gradient 20-35% B in 105 minutes.
Diluted crude cyclic to 15% ACN (pH=4.5) before loading
Performed 3 runs with different column loading.
Results are described below

TABLE 14
RPC1 Purification test

| | | | Est. | Est. | | | | Amt in | Main | |
|---|---|---|---|---|---|---|---|---|---|---|
| | Column | Linear | Crude | Product | Amt. | Crude | | Main | pool | |
| | Diameter | Crude | (mg/g | (mg/g | Loaded | Purity | Elution | Pool | Purity | Recovery |
| Exp | (cm) | (g) | media) | media) | (g) | (%) | (% B) | (mg) | (%) | (%) |
| 1 (BPR) | 1 | 0.5 | 23 | 0.83 | 0.05 | 50.3 | 30 | 40 | 92.57 | 80 |
| 2 | 2.5 | 4.5 | 60 | 13.3 | 1.0 | 50.3 | 30 | 821 | 91.13 | 82% |
| 3 | 2.5 | 5.0 | 66.7 | 26.7 | 2.0 | 51.67 | 27 | n/a | n/a | n/a |

### Conclusion

The current RPC1 method was used to purify the crude from the less optimized cyclization experiments. With the optimized synthesis and cyclization processes, loading amount of crude can be doubled (2.6 times) without any impact on purity and recovery. Column was overload when loading was tripled.

### RPC 2 Purification Test

The current RPC2 method was subjected for the study. The fractions with purity>85% from the 0.1 M NH₄OAc (aq.) were combined. Lower sample purity (side cuts) was used to test the purification power of this method.
Column media: Daiso Gel C18 120 Å, 10 m
Column diameter: 2.5 cm
Loaded amount˜1.56 g, purity=88.04, SLI=3.76%
Sample was diluted equal volume of water before loading
Buffer A was 0.1% TFA (aq.)
Buffer B was acetonitrile.
The gradient used was 15% B to 35% B in 100 minutes.
The product eluted at 29% B
collected 15 mL fractions.

### Results and Discussion

Main pool Purity=95.64, SLI=1.43%, Amount=827.2 mg with a recovery of 53% was obtained. This result showed that the current RPC2 method could be used to purify the product (RPC1 main pool) with purity≤90% but the recovery would be negatively impacted. Therefore, the RPC1 main pool purity≥90% criterion was chosen to be used for RPC2 purification.

### RPC3 Development

This stage (RPC3) was added for TFA reduction in the final lyophilized product. Since the high TFA content negatively impact the stability of the lyophilized product. The salt selection work was performed first.

### Salt Selection Experiments

Two runs for salt selection work were performed, and the resulting main pools were lyophilized and tested for stability. The runs were following: (a) loading the TFA salt, wash and elute with 30% ACN/water, and (b) loading the TFA salt, salt exchange with 0.1 M NH₄Cl, pH=4.5 and elude with 30% ACN/water

TFA Salt column Wash and Elution
Column: Daiso Gel C18, 120 Å, 15 m, 0.46×1 cm
Conditioned column with 2 BV of 5% ACN/0.1% TFA
Loaded 113 mg of lyophilized product dissolved 10 of 5% ACN/0.1% TFA
Passed 2 BV of 5% ACN in water
Gradient: 5% B-10% B in 10 minutes, then 10% B-30% B in 10 mins
Mobile phase A: water; mobile phase B: ACN
Product eluted after 2 BV of 30% B
Main pool pH=6.5, lyophilized to recover 76.6 mg; Purity=94.81% (TFA method)
Ammonium Chloride to Chloride Experiments
Column: Daiso Gel C18 120 Å 15 m, 0.46×1 cm
Conditioned column with 2 BV of 5% ACN/0.1% TFA
Loaded 116 mg of lyophilized product dissolved 10 of 5% ACN/0.1% TFA
Passed 3 BV of 0.1 M ammonium chloride/5% ACN
Passed 1 BV of 5% ACN in water
Gradient: 5% B-10% B in 10 minutes, then 10% B-30% B in 10 mins
Mobile phase A: water; mobile phase B: ACN
Product eluted after 2 BV of 30% B
Main pool pH=6.81, lyophilized to recover 81 mg; purity=94.99% (TFA method)

### Conclusion

Samples from each run were given to analytical development for analysis. The salt content and stability were tested. Both samples were found to contain no counter ions, meaning that the product was in its free base form. The stability was found to similar for both samples and better than the original TFA salt. The TFA column wash was selected for further development.

### RPC 3 Purification Development

The RPC2 main pool was desalted and further purified in this stage. The purification method was developed on the same media that was used for RPC1 and RPC2 with the same flow rate. The experiment is described below:
Column media: Daiso Gel C18 120 Å, 10 m
Column diameter: 2.5 cm
Buffer A=water, buffer B=ACN
The column is conditioned with 2 BV of 5% ACN/0.1% TFA
RPC2 main pool purity=95.64, SLI=1.43%, amount=827.2 mg diluted with equal vol. water and loaded
Passed 2 BV of 10% ACN/water to remove TFA
Gradient 20-35% B in 60 minutes.
Eluted at 32.5% B
collected 20 mL fraction.
Results: main pool purity=95.91, SLI=1.21%, amount=800 mg
Recovery=96.7%

This method is selected for large scale demonstration.

### Large Scale Purification and Lyophilization

### 0.1 M NH₄OAc Purification (RPC1)

The cyclic crude solution was filtered through a 2.4 μm filter and loaded on the preparative reversed-phase column. The purification method used is described below.
0.1 M NH₄OAc purification conditions (RPC 1)
Column diameter: 10 cm
Column media: Daiso Gel™ C18, 120 Å, 10 μm
Quantity of media packed: 1.2 kg
Buffer A: 0.1 M NH₄OAc/H2O, buffer B: 100% ACN
Gradient: 10-20% buffer B in 10 minutes, then 20-35% buffer B in 105 minutes
Flow rate: 175 mL/min.
Wavelength: 230 nm

### Procedure

Pass 2 bed volumes 5% ACN in 0.1% TFA (aq.).
Filter the resulting solution through 2.4 μm filter.
Load sample onto column
Pass 1 bed volume of 10% buffer B
Start the gradient as stated above.
Collect fractions (˜250 mL) when the product start to elute.
Backwash the column with 3 BV of 80% MeOH in water.

### Result and Discussion

The cyclic crudes from were purified. The pH of the crude sample #1 was pH=4.5, while the second crude was pH=6.8. the result of the runs was summarized below.

TABLE 15
RPC1 using Gradient of 15% B to 37% B in 110 minutes

| | | Ext. | | | | |
|---|---|---|---|---|---|---|
| | | Product | Sample | Est Product | | |
| | | content | Purity | in Main pool | Purity | SLI |
| RPC1 # | Sample | (g) | (%) | (g) | (%) | (%) |
| 1 | Crude | 18 | 74.32 | 15 | 93.93 | 2.40 |
| 2 | Crude | 18 | 72.48 | 15 | 93.96 | 2.42 |

The recovery from this purification stage was 83%. The main pool hold time is reported in section 11.

### TFA Purification (RPC2)

The main pools from the 0.1 M NH₄OAc (aq.) purification were diluted with equal volume of water and loaded onto the same column. Then 5% ACN in 0.1% TFA (aq.) was passed through the column to facilitate salt exchange. The purification and elution of the product with 0.1% TFA (aq.) was performed using the conditions stated below.

### 0.1% TFA Conditions (RPC2)

Column diameter: 10 cm
Column media: Daiso Gel™ C18, 120 Å, 10 μm
Quantity of media packed: 1.2 kg
Buffer A: 0.1% TFA in water, buffer B: 100% ACN
Gradient: 5-15% B in 10 min., then 15-35% buffer B in 100 minutes
Wavelength: 230 nm
Flow rate: 175 mL/min.
Product eluted at about 29% buffer B

### Procedure

Pass 2 bed volumes 5% acetonitrile in 0.1% TFA in water.
Dilute the NH₄OAc main pool with equal volume of water.
Load the diluted main pool onto the column
Pass 2 bed volume 10% buffer B
Run gradient as specified in RPC2 condition
Collect fractions (˜300 mL) when the product start to elute.
Backwash the column with 3 BV of 80% MeOH in water.

The amount loaded to the column was ˜30 g (25 g/kg of column media). About 23 g (77%) of the estimated product loaded (RPC1-main pool by peak area) which was loaded on the column for RPC2 purification, was recovered with HPLC purity of 95.22% and single largest impurity of 1.43% (see FIG. 18 below). No side cut was processed in this stage. The TFA main pool was stable at 5° C. for 28 days (section 11).

### TFA Desalting (RPC3)

The main pool from the 0.1% TFA (aq.) purification was diluted with equal volume of water and loaded onto the same column. Then 10% ACN in purified water was passed through the column to desalt the TFA salt. The purification and elution of the product with purified water and ACN was performed using the conditions stated below.

### Desalting Conditions (RPC3)

Column diameter: 10 cm
Column media: Daiso Gel™ C18, 120 Å, 10 μm
Amount of media packed: 1.2 kg
Buffer A: water, buffer B: 100% ACN
Gradient: 10-20% B in 10 min., then 20-35% buffer B in 60 minutes
Wavelength: 230 nm
Flow rate: 175 mL/min.
Product eluted at about 32% buffer B

### Procedure

Pass 2 bed volumes 5% acetonitrile in 0.1% TFA in water.
Dilute the TFA main pool with equal volume of water.
Load the diluted main pool onto the column
Pass 2 bed volume 10% buffer B
Run gradient as specified in RPC3 condition
Collect fractions (˜500 mL) when the product start to elute.
Backwash the column with 3 BV of 80% MeOH in water.

An estimated 23 g with HPLC purity of 95.22% and single largest impurity of 1.43% was loaded to the column and about 10 g with purity=95.91, SLI=1.49% was in the main pool. This mean that no purification was observed, with recovery of only 43%. This shows that the result observed in the 2.5 cm column purification could not be reproduced. To see if this is due to scalability or just poor column performance, a 5 cm column was employed to repeat this experiment.

### RPC3 Sidecut Processing

The RPC3 side cut solution (˜11 g) was diluted with equal volume of water and loaded to the column.

### Desalting Conditions (RPC3)

Column diameter: 5 cm
Column media: Daiso Gel™ C18, 120 Å, 10 μm
Amount of media packed: 300 g
Buffer A: water, buffer B: 100% ACN
Gradient: 10-20% B in 10 min., then 20-40% buffer B in 50 minutes
Wavelength: 230 nm
Flow rate: 43.7 mL/min.
Product eluted at about 32% buffer B

### Procedure

Pass 2 bed volumes 5% acetonitrile in 0.1% TFA in water.
Dilute the side cut with equal volume of water.
Load the diluted main pool onto the column
Pass 2 bed volume 10% buffer B
Run gradient as specified in RPC3 condition
Collect fractions (˜50 mL) when the product start to elute.
Backwash the column with 3 BV of 80% MeOH in water.

Only 6 g was recovered from possible 11 g (54% recovery). This confirms that the result from the 2.5 cm column desalting run is not scalable. A kickout experiment with a fast gradient is needed.

### Desalting and Kickout

All the fractions from section 9.2.3.1 were combined, diluted with equal volume of water and reloaded to the column for this experiment.

### Desalting Conditions (RPC3)

Column diameter: 5 cm
Column media: Daiso Gel™ C18, 120 Å, 10 μm
Amount of media packed: 300 g
Buffer A: water, buffer B: 100% ACN
Gradient: 10-35% B in 10 min., then hold at 35% buffer B till all product is eluted
Wavelength: 230 nm
Flow rate: 43.7 mL/min.
Product eluted at about 32% buffer B

### Procedure

Pass 2 bed volumes 5% acetonitrile in 0.1% TFA in water.
Dilute the side cut with equal volume of water.
Load the diluted main pool onto the column
Pass 2 bed volume 10% buffer B
Run gradient as specified in RPC3 condition
Collect fractions (˜50 mL) when the product start to elute.
Backwash the column with 3 BV of 80% MeOH in water.

Approximately 10.8 g was recovered from the 11 g (36.7 g/kg of column media) loaded, so it is safe to say that all the product loaded was recovered. The concentration of the final main pool was 25 g/L. This procedure will be recommended for large scale desalting.

### Lyophilization

The main pools from desalting runs were combined and lyophilized in bottles. After lyophilization, 24 g of final lyophilized product was collected. The purity of the final lyophilized product was found to be 95.77% with a single largest impurity of 1.49%, and the overall yield was ˜10.6%.

### Hold Time Study

Hold times studies were performed on the final solutions for each stage starting from cyclization. The conditions were ambient temperature (sample left in the room) and 2-8° C. (stored in a refrigerator).

### Crude

Hold time study were performed for the crude at pH=4.5 and pH=6.8. The summary of these study is shown in tables below.

TABLE 16
Hold time result for Crude, pH = 4.5

| | | |
|---|---|---|
| Crude, pH = 4.5 | RT | 5° C. |

| Week | Purity | SLI | Purity | SLI |
|---|---|---|---|---|
| 0 | 69.63 | 3.54 | n/a | n/a |
| 1 | 68.22 | 2.13 | 69.19 | 2.78 |
| 2 | 70.88 | 2.94 | 65.81 | 2.99 |
| 3 | 70.36 | 2.86 | 64.20 | 3.11 |
| 4 | 66.20 | 2.96 | 68.96 | 3.05 |

TABLE 17
Hold time result for Crude, pH = 6.8

| | | |
|---|---|---|
| Crude pH = 6.8 | RT | 5° C. |

| Week | Purity | SLI | Purity | SLI |
|---|---|---|---|---|
| 0 | 69.82 | 4.29 | N/A | N/A |
| 1 | 71.63 | 8.31 | 74.59 | 2.21 |
| 2 | 60.33 | 18.88 | 68.33 | 4.08 |
| 3 | 59.92 | 27.29 | 66.46 | 7.39 |
| 4 | 51.64 | 24.16 | 64.82 | 10.46 |

The crude with pH=4.5 is more stable at both conditions and can be left at room temperature for 3 weeks and refrigerated for a month. At pH=6.8, the crude can be stored at room temperature for 1 week and refrigerated for 2 weeks.

### RPC1 Main Pool

The ammonium acetate main pool was stored at room temperature, and purity monitored weekly. The results are summarized below

TABLE 18
RPC1 Main pool Hold Time Result

| | | |
|---|---|---|
| RPC1 | RT | 5° C. |

| Week | Purity | SLI | Purity | SLI |
|---|---|---|---|---|
| 0 | 93.93 | 2.08 | N/A | N/A |
| 1 | 93.50 | 1.68 | 93.58 | 1.74 |
| 2 | 93.27 | 1.25 | 93.47 | 1.68 |
| 3 | 92.86 | 1.64 | 93.25 | 1.69 |
| 4 | 93.15 | 1.67 | 94.39 | 1.31 |

The main pool in this stage can be stored at room temperature or refrigerated for one month.

### RPC2 Main Pool

The TFA main pool was stored at room temperature, and purity monitored weekly. The results are summarized below.

TABLE 19
TFA Main pool Hold Time Result

| | | |
|---|---|---|
| RPC2 | RT | 5° C. |

| Week | Purity | SLI | Purity | SLI |
|---|---|---|---|---|
| 0 | 95.44 | 2.02 | n/a | n/a |
| 1 | 94.54 | 2.17 | 94.96 | 1.96 |
| 2 | 93.37 | 1.73 | 95.67 | 1.83 |
| 3 | 92.02 | 1.75 | 95.17 | 1.97 |
| 4 | 91.62 | 2.80 | 95.26 | 2.01 |

The TFA main pool should be refrigerated for up to 1 month. Room temperature storage is not recommended.

### RPC3 Main Pool

The final desalted solution before lyophilization was studied for stability. The results are reported in the table below.

TABLE 20
Desalted Solution Hold Time Result

| | | |
|---|---|---|
| RPC3 | RT | 5° C. |

| Week | Purity | SLI | Purity | SLI |
|---|---|---|---|---|
| 0 | 96.2 | 1.99 | n/a | n/a |
| 1 | 95.81 | 2.01 | 95.89 | 1.99 |
| 2 | 95.82 | 1.98 | 95.81 | 1.97 |
| 3 | 95.69 | 2.00 | 95.80 | 1.98 |
| 4 | 95.63 | 2.07 | 96.29 | 1.75 |

Both room temperature and refrigerated solutions are stable for one month.

### Conclusion

### Synthesis

The SPPS of BCY8234 was optimized to minimize the formation of aspartimide impurities.

Different deblocking cocktails were studied using a DOE style screening experiments.

The deblocking cocktail containing 3% oxyma in 10% piperidine/DMF slightly outperformed 3% oxyma in 5% piperidine/DMF and 5% oxyma in 10% piperidine.

3% oxyma in 10% piperidine/DMF was selected for GMP manufacturing.

DITU was added to the coupling solutions to help suppress cysteine oxidation.

Sarcosine dipeptide derivative was used for the sarcosine couplings.

Higher loading (>0.8 mmol/g) resin was successfully used for this optimization work.

### Cleavage

A series of cleavage experiments were performed.

1,4-BDMT was compared to DTT and the result shows no significant difference between them.

Minitab was used to design screening experiments using DTT

The result of the screening experiment presented two possible cocktail choices, the Minitab response optimizer's choice and the choice of the team.

Further optimization work revealed the optimal method to be: 90% TFA, 15% DTT, 5% water cooled to 10° C., before addition of resin, then 5% TIPS added after 1 h.

The cocktail to resin ratio is 10 mL/g and the reaction performed for 3 h at RT.

Precipitation was performed with −40° C. (4×TFA cocktail), filtered and washed 3× time with MTBE

150 g was cleaved and recovered 142 g with Purity=74.04%

### Cyclization

A series of optimization experiments were performed for cyclization

Key findings were as follows:
TATA can be reduced to 1.3 eq.
The reaction time can be reduced to 4 hours and
The ACN content in reaction solution can be reduced to 20%.
Two 121 g cyclization were performed to generate an estimated 18 g of product.
The crude cyclic solution is loaded to the column at pH=6.8
Longer crude storage requires a pH=4.5

### Purification

0.1M NH₄OAc (aq.) was used to purify the crude cyclic peptide.

Then 0.1% TFA (aq.) was employed for further purification and final TFA salt generation.

The purified TFA salt was desalted via water wash and eluted with 35% ACN/water.

Without wishing to be bound by any particular theory, it is believed that that the main advantage of desalting is in the long-term stability of solid peptide intermediate free from acidic or basic counterions.

### Lyophilization

The desalted TFA best pool was bottle lyophilized to obtain 24 g of product with purity of 95.77% and single largest impurity of 1.49%.

### Recommendations for GMP Production

### Synthesis

3000 g of rink amide MBHA resin (2.4 moles)
The reaction vessel must be under inert condition (N₂ or Argon) at all times.
After coupling of Asp19, all deblocking must use 3% oxyma in 10% piperidine/DMF
All deblocking times must be 5 and 20 minutes.
Avoid leaving the piperidine solution in the vessel for an extended time (≤5 minutes combined drain time is ideal.)
0.2 eq of DITU should be added to coupling solutions
All sarcosine coupling must be done with Fmoc-Sar-Sar-OH

Without wishing to be bound by any particular theory, it is believed that the main advantage of using Fmoc-Sar-Sar-OH for all sarcosine couplings is to reduce the number of peptide synthesis and deprotection cycles while maintaining overall coupling efficiency on solid phase thus minimizing opportunities for aspartimide formation.

### Cleavage

A range of 1000 to 2000 g peptide-resin is recommended
Cleavage and global deprotection of the peptide to be performed by treating the resin with a cocktail (10 mL/g) of 90% TFA, 5% water, 0.25% NH₄I, 5% DTT and 5% TIS (added after 1 hour) for 3 hours.
The reaction mixture with spent resin will be precipitated with cold MTBE, and the resultant precipitate isolated and dried.

### Cyclization

500 g linear crude+spent resin recommended.
40 g of TATA pre-weighed into a 5 L screw cap flask
TATA weighing and dispensing must be performed in an isolation system.
Exposure to TATA must be very minimal
Reaction solution is 66 L of 0.1 M NH₄HCO₃ in 20% ACN (aq.)
Reaction must be 4-20 hours.
Hold time at 2-8° C.=5 days (after acetic acid treatment, pH=4.28)

### Purification

20 cm column packed with Daiso Gel C18 10 m 120 Å
Buffer B=ACN
The current BPR will be retained for RPC1 & RPC2.
In RPC3, three BV of 10% ACN/water used to wash out residual TFA and the product eluted with 35% ACN/water.

### HPLC Conditions and Certificate of Analysis

TABLE 21
Analytical HPLC Conditions for Cleavage and Cyclization

| Column size | 150 × 4.6 | mm |
|---|---|---|
| Support | Cortecs C18, 2.7 μm | |
| Flow rate | 1.0 | mL/min |
| Wavelength | 214 | nm |
| Temperature | 50° | C. |

| | |
|---|---|
| Gradient | 20-40% B in 40 min |
| Buffer A | 0.1% TFA/H2O |
| Buffer B | 0.1% TFA/ACN |

TABLE 22
Analytical HPLC Conditions for Purification and Lyophilization

| Column size | 150 × 4.6 | mm |
|---|---|---|
| Support | Meteoric Core C18, 2.7 μm | |
| Flow rate | 0.8 | mL/min |
| Wavelength | 214 | nm |
| Temperature | 60° | C. |

| | |
|---|---|
| Gradient | 26-34% B in 32 min |
| Buffer A | 0.1M NaClO₄ pH = 3.5 |
| Buffer B | ACN |

### Certificate of Analysis

BCY8234
Item Number: 512175
Lot Number: P200462
Molecular Weight: 2954.3
Appearance: White Powder
Peptide Purity: ≥95%
Peptide Content: 92.8% (by nitrogen content)
Water (KF): 7.1%
TFA: none detected
Mass balance: 99.9%
Identity: Mass spectral analysis (ESI) exhibits correct molecular ion (2953.3)
Storage: Keep dry and store below −20° C.

### REFERENCES

Acid-Mediated Prevention of Aspartimide. Michels, Tillmann, et al. 2012, ORGANIC LETTERS, Vol. 14, No. 20, pp. 5218-5221.
ReGreen SPPS: enabling circular chemistry in. Pawlas, Jan: Rasmussen Jon H. 2019, Green Chemistry (21), pp. 5990-5998.

### Example 2: Preparation of BT8009

### Introduction

Goal: develop a new process for BT8009 production at kilo lab scale. This example describes process development activities conducted to address the issues identified from process development.

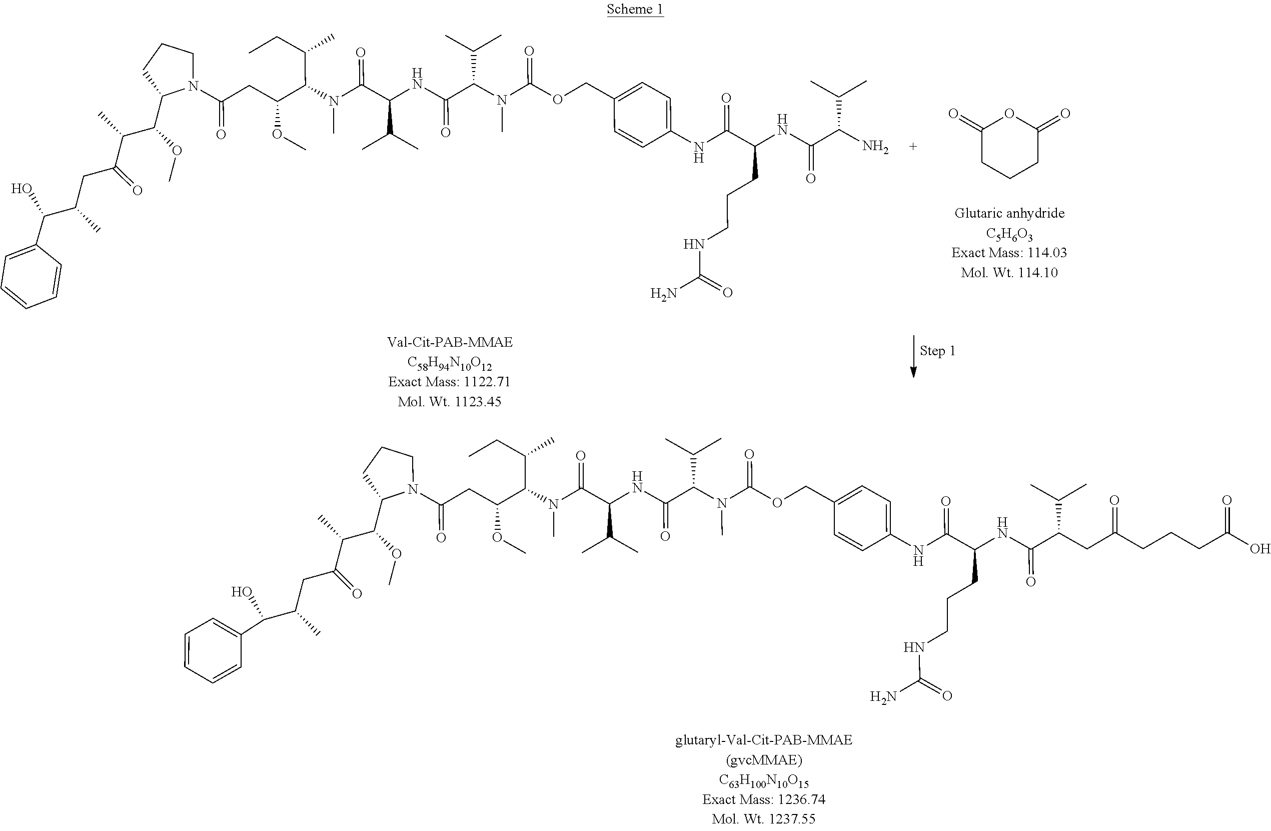 [SMILES: CC[C@H](C)[C@@H]([C@@H](CC(=O)N1CCC[C@H]1[C@H](OC)[C@@H](C)C(=O)C[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)C[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](CC(=O)CCCC(=O)O)C(C)C)cc1)C(C)C]

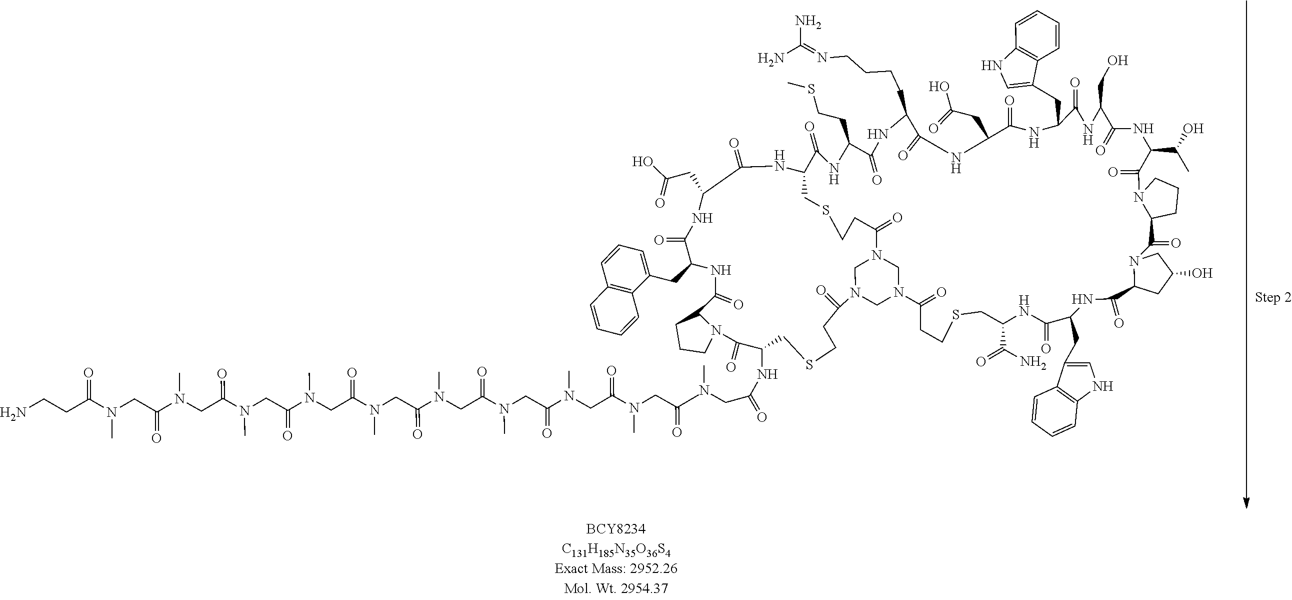

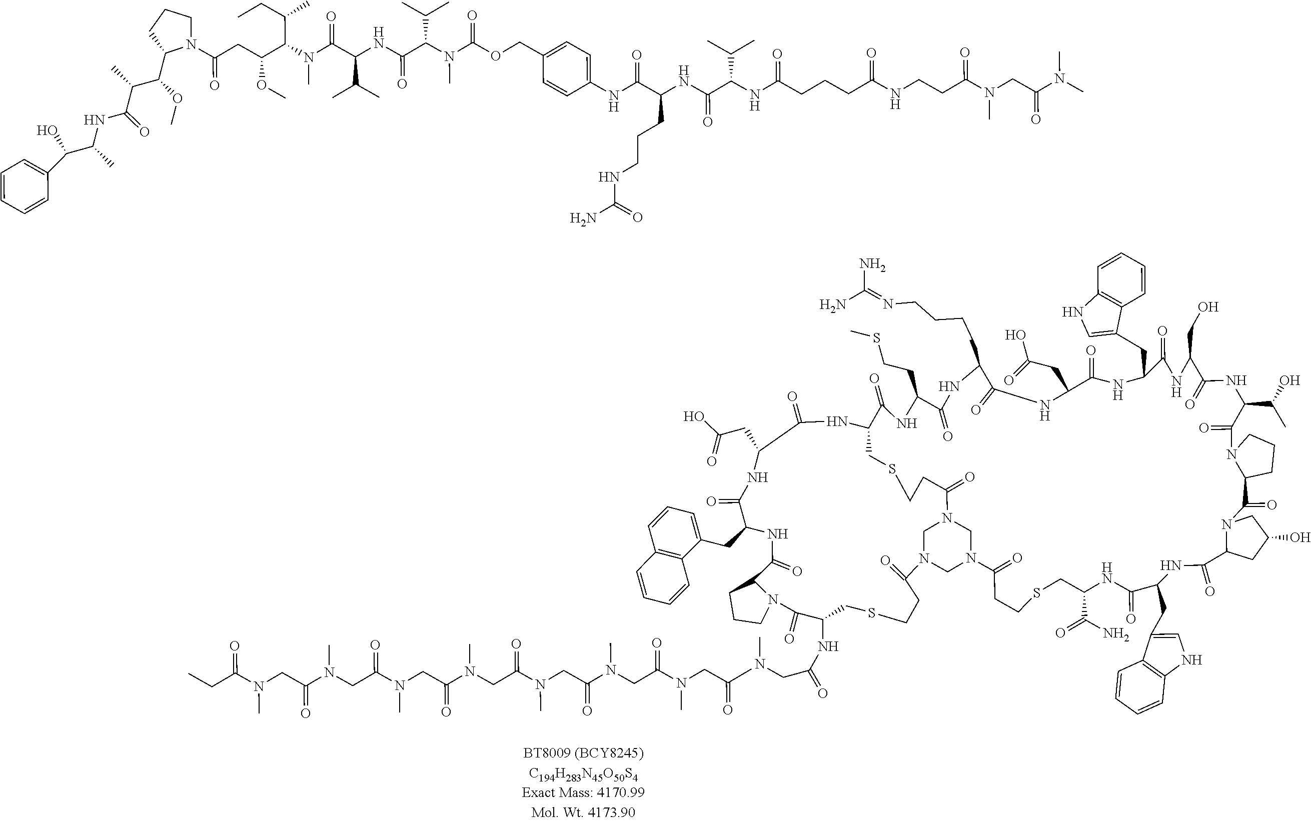

### Results and Discussion

### Step 1: Formation of gvcMMAE

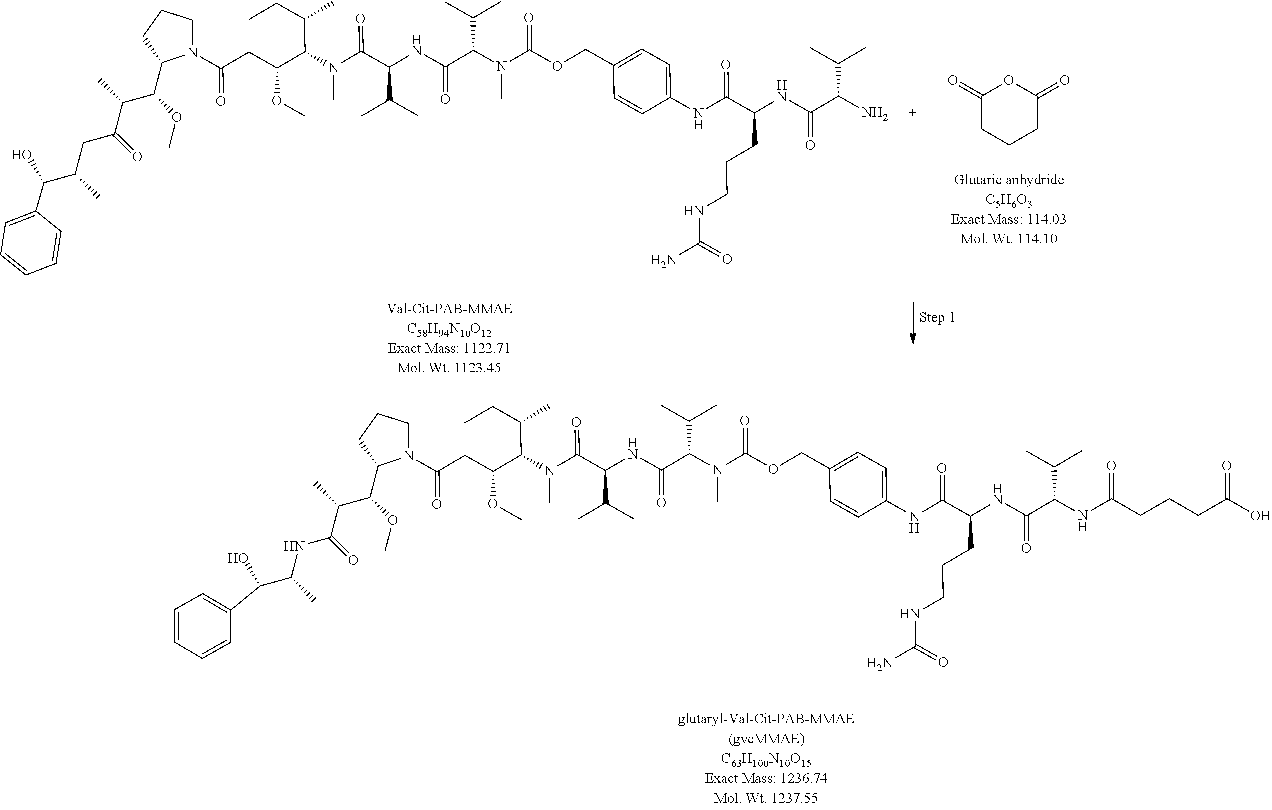 [SMILES: CC[C@H](C)[C@@H]([C@@H](CC(=O)N1CCC[C@H]1[C@H](OC)[C@@H](C)C(=O)C[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

Five experiments were performed on 1-3 g scale (Table 23). Entry 1 experiment was run to simplify the workup procedure and improve the yield. The 1:1 EtOAc/THF was used to extract the brine solution. Both organic and aqueous layers contained a lot of gvcMMAE. Extraction of the aqueous reaction solution with EtOAc/THF was not successful. Therefore, the reaction solution was charged into an acidic brine solution. A filterable suspension was obtained. The product was obtained with 94.3% LC purity and 88% yield. There was only 0.07% w/w of sodium chloride in the product.

Entry 2 experiment was run to reduce the workup volume. The workup volume was reduced from 70 to 50 volume. The reaction solution was charged into a HCl acidic water solution. The product precipitated out of the solution. The product was obtained with 93.5% LC purity and 79% yield. The aqueous solution dissolved more gvcMMAE than brine solution and resulted in lower yield.

Entry 3 experiment was run to investigate the reason why entry 2 experiment had a lower yield than entry 1. In entry 3 experiment, entry 1 experimental procedure was repeated except that water replaced brine in the workup. The product was obtained with 94.6% LC purity and 72% yield. The result indicated that the brine was critical to achieve a higher yield.

Entry 4 experiment was run to continue investigating the reason why entry 2 experiment had a lower yield than entry 1. In entry 4 experiment, entry 1 experimental procedure was repeated except that the reaction mixture was distilled to remove DIPEA. The product was obtained with 94.6% LC purity and 74% yield. The result indicated that the distillation was not critical to achieve a higher yield.

The results of entry 1-4 has demonstrated that the brine is critical to achieve a higher yield. Entry 5 experiment was performed to confirm this hypothesis. The reaction solution was charged into an acidic saturated brine solution. The suspension was filtered. The product was obtained with 95400 LC purity and 910 yield after assay adjustment. There was only 0.45 w/w of sodium chloride in the product. These conditions will be used as a preferred procedure (see attachments).

TABLE 23
Step 1 results

| | | | Val-Cit- | | | | |
|---|---|---|---|---|---|---|---|
| | | | PAB- | | | | |
| Entry | Experiment | | MMAE | Workup | IPC | Yield | Purity |
| 1 | BC9-PD-0001 | | 3 g | Charged reaction solution to acidic | 1 h: | 88%* | 94.3% |
| | | | | 13% brine solution (70 vol) | 0.03% SM | | |
| 2 | BC9-PD-0003 | | 3 g | Charged reaction solution to acidic | 1 h: ND | 79% | 93.5% |
| | | | | water solution (50 vol) | | | |
| 3 | BC9- | −1 | 1 g | Charged reaction solution to acidic | 1 h: ND | 72% | 94.6% |
| | LHE- | | | water solution (70 vol) | | | |
| 4 | 0001 | −2 | 1 g | Distilled reaction solution, charged | | 74% | 94.6% |
| | | | | reaction solution to acidic water | | | |
| | | | | solution (70 vol) | | | |
| 5 | | −3 | 1 g | Charged reaction solution to acidic | | 91%* | 95.4% |
| | | | | saturated brine solution (70 vol) | | | |

*IC indicates the product contains little NaCl

### Step 2: Formation of BT8009

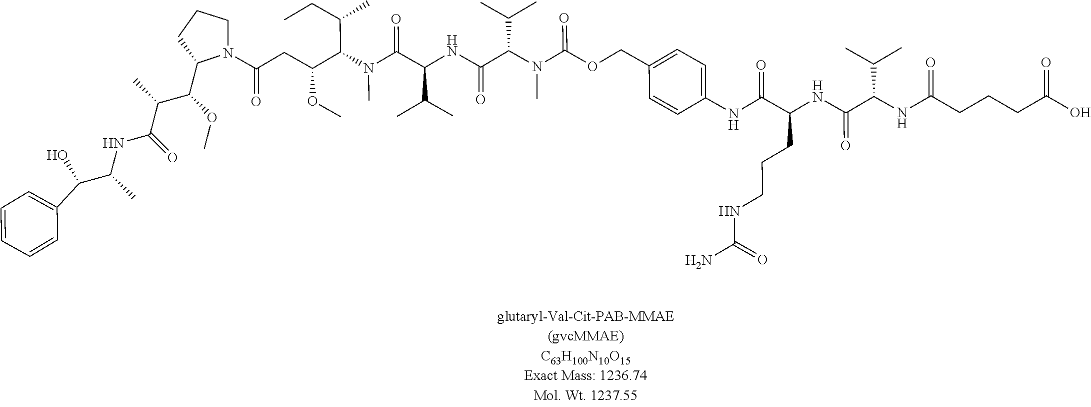 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

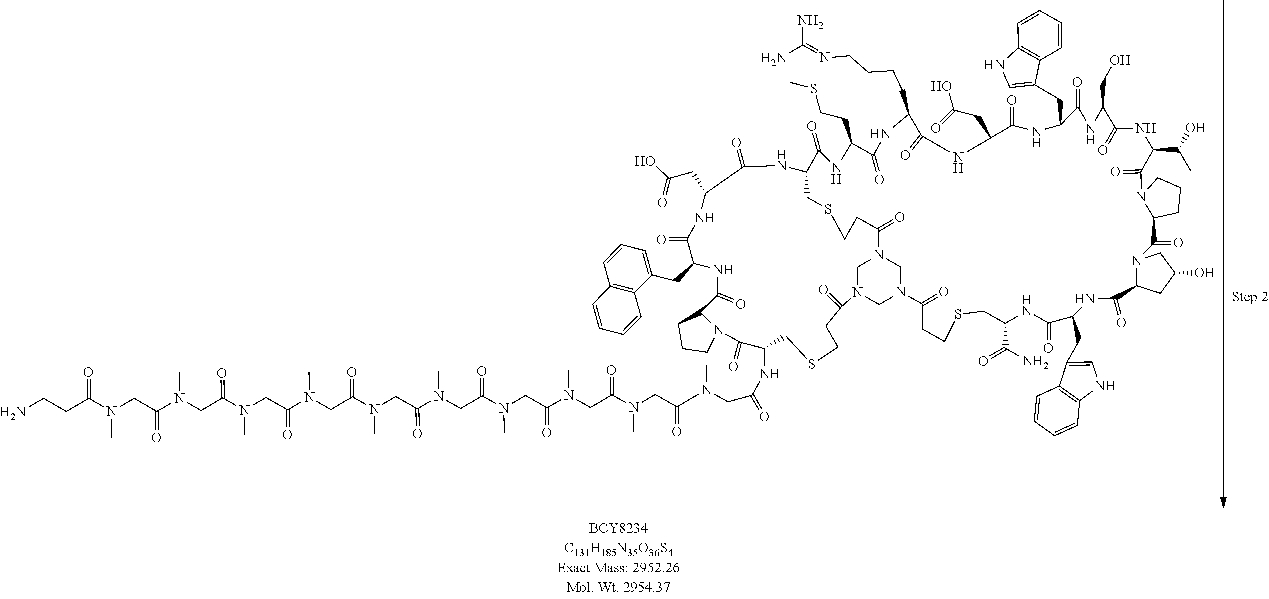

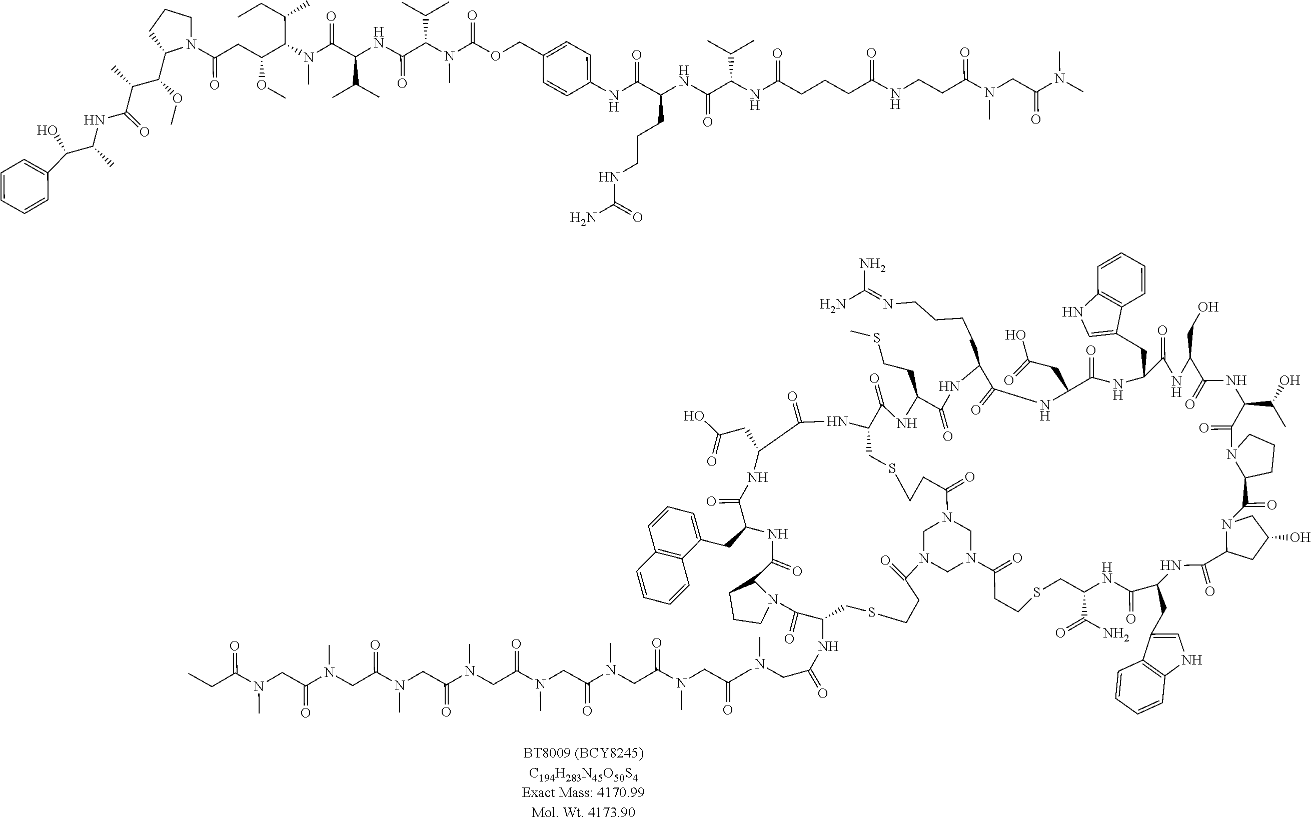

Eleven experiments were performed on 0.708-2.832 g scale (Table 24). Two lots of BCY8234 were utilized in these experiments where Lot C was prepared by an earlier route and lot P by a later route. Entry 1 experiment was run to explore a workup procedure and make adequate crude BT8009 to optimize the column purification condition. The BCY8234 starting material was from Lot C and had 7.62% w/w of TFA. This BCY8234 was easy to dissolve in DMA. After stirring the reaction for 1 hour, IPC showed 1.73% BCY8234, 0.75% gvcMMAE and 0.07% RRT 0.93 impurity. The reaction solution was charged into an MTBE solution. The product was precipitated out as a filterable suspension. The suspension was filtered through a class D funnel. The assay analysis indicated there was no product in the filtrate. To avoid forming a sticky solid, the solvent was kept above the cake during filtration. When the rinse was completed, and the solvent stopped dripping, the vacuum was stopped immediately. The crude product was obtained with 86.1% LC purity and assuming 100% yield.

Entry 2-4 experiments were performed to explore the column purification conditions. In each experiment, 1 g of theoretical BT8009 was pulled from entry 1 crude product and purified by a 60 g ultra C18 column.

In entry 2 experiment, 10-40% ACN/H₂O plus 0.1% AcOH was used for the gradient elution of an ultra C18 column. After lyophilization, BT8009 was obtained with 96.2% LC purity and no RRT 0.93 impurity and 89.8% yield.

In entry 3 experiment, 10-40% ACN/H₂O plus 0.05% AcOH was used for a gradient elution of an ultra C18 column. After lyophilization, BT8009 was obtained with 95.6% LC purity and no RRT 0.93 impurity and 61.1% yield.

In entry 4 experiment, 10-35% ACN/H₂O plus 0.1% AcOH was used for a gradient elution of an ultra C18 column. After lyophilization, BT8009 was obtained with 96.9% LC purity and 0.10% of RRT 0.93 impurity and 62.7% yield. The results indicated that entry 2 purification was the best condition but needed to be optimized.

Entry 5 experiment was run to confirm if the BCY8234 from Lot P would provide acceptable final product. In this experiment, 5 equivalents of DIPEA and 1 equivalent of gvcMMAE/TBTU were used. This BCY8234 had no TFA and was not dissolved in DMA. After stirring the suspension for 1 hour, IPC showed 36.14% of BCY8234 and 2.78% of gvcMMAE and 2.81% RRT 0.93 impurity. After additional charges (2×0.1 equivalent) of gvcMMAE/TBTU, IPC showed 3.32% of BCY8234 and 3.55% of gvcMMAE and 3.88% RRT 0.93 impurity.

Entry 6 experiment was run to repeat entry 5 experiment except using 11 equivalents of DIPEA to see if the reaction would be improved. The IPC was like that of entry 5. After stirring the suspension for 1 hour 13 minutes, IPC showed 27.33% of BCY8234 and 3.76% of gvcMMAE and 1.19% RRT 0.93 impurity. After additional charges (3×0.1 equivalent) of gvcMMAE/TBTU, IPC showed 0.16% of BCY8234 and 4.37% of gvcMMAE and 1.66% RRT 0.93 impurity.

In entry 7 experiment, the BCY8234 from Lot P was dissolved in DMA and 4 equivalents of TFA before mixing with gvcMMAE/TBTU. After stirring for 17 hours, IPC showed 4.62% of BCY8234 and 0.99% of gvcMMAE and 0.38% of RRT 0.93 impurity. After an additional charge (0.1 equivalent) of gvcMMAE/TBTU, IPC showed 0.26% of BCY8234 and 1.53% of gvcMMAE and 0.79% of RRT 0.93 impurity. In this experiment, 10-40% ACN/H₂O plus 0.1% AcOH was used for a gradient elution of an ultra C18 column. After lyophilization, BT8009 was obtained with 94.7% LC purity and 0.99% of RRT 0.93 impurity and 59.1% yield.

In entry 8 experiment, the BCY8234 from Lot P was dissolved in DMA and 3 equivalents of TFA and 12 equivalents of water before mixing with gvcMMAE/TBTU. After stirring for 1 hour, IPC showed 3.82% of BCY8234 and 1.14% of gvcMMAE and 0.38% of RRT 0.93 impurity. After an additional charge (0.1 equivalent) of gvcMMAE/TBTU, IPC showed 0.21% of BCY8234 and 1.98% of gvcMMAE and 1.69% of RRT 0.93 impurity. In this experiment, 10-38% ACN/H₂O plus 0.1% AcOH was used for a gradient elution of an ultra C18 column, 45% ACN/H₂O plus 0.1% AcOH was used to ensure all product was eluted off the C18 column. A catch-release column was performed. After lyophilization, BT8009 was obtained with 95.5% LC purity and 1.36% of RRT 0.93 impurity and 68.5% yield.

Entry 9-10 experiments were performed to check if direct charge of 1.1 equivalents of gvcMMAE/TBTU would minimize the RRT 0.93 impurity. In entry 9 experiment, the BCY8234 from Lot P was used for the reaction. After stirring for 1 hour, IPC showed 0.55% of BCY8234, 1.72% of gvcMMAE and 1.04% of RRT 0.93 impurity. In entry 10 experiment, the BCY8234 from Lot C was used for the reaction. After stirring for 1 hour, IPC showed 0.23% of BCY8234, 1.68% of gvcMMAE and 0.87% of RRT 0.93 impurity. The results indicated that excessive gvcMMAE/TBTU resulted in the RRT 0.93 impurity, and 1 equivalent of gvcMMAE/TBTU should be used for step 2 reaction. This impurity was generated from both Lot P and Lot C BCY8234.

In entry 11 experiment, the BCY8234 from Lot P was dissolved in DMA and 4 equivalents of TFA before mixing with gvcMMAE/TBTU. One equivalent of gvcMMAE/TBTU was used in this reaction. After stirring for 1 hour, IPC showed 3.45% of BCY8234, 2.00% of gvcMMAE and 0.01% of RRT 0.93 impurity. After lyophilization, BT8009 was obtained with 96.9% LC purity, no RRT 0.93 impurity and 64.7% yield. This experiment will be used as a preferred procedure.

TABLE 24
Step 2 results

| Entry | Experiment | BCY8234 | DIPEA | | gvc-MMAE | | IPC | Workup | Yield | Purity |
|---|---|---|---|---|---|---|---|---|---|---|
| 1 | BC9-PD- | 2.832 g, | 11 | eq | 1 | eq | 1 h: 1.73% | Charged | | |
| | 0002 | Lot C, | | | | | BCY8234, | reaction | | |
| | | 1909118, | | | | | 0.75% | solution | | |
| | | 97.9% | | | | | gvc MMAE, | to MTBE | | |
| | | | | | | | RRT | solution | | |
| | | | | | | | 0.93:0.07% | | | |
| 2 | BC9-PD- | | | | | | | Column: | 89.8% | 96.2% |
| | 0002-1 | | | | | | | 10-40% | | |
| | | | | | | | | ACN/H₂O | | |
| | | | | | | | | 0.1% AcOH | | |
| 3 | BC9-PD- | | | | | | | Column: | 61.1% | 95.6% |
| | 0002-2 | | | | | | | 10-40% | | |
| | | | | | | | | ACN/H₂O | | |
| | | | | | | | | 0.05% AcOH | | |
| 4 | BC9-PD- | | | | | | | Column: | 62.7% | 96.9% |
| | 0002-3 | | | | | | | 10-35% | | |
| | | | | | | | | ACN/H₂O | | |
| | | | | | | | | 0.1% AcOH | | |
| 5 | BC9-LHE- | 1.416 g, | 5 | eq | 1 + 0.1 + 0.1 | eq | 55 h: 3.32% | | | |
| | 0002 | Lot P, | | | | | BCY8234, | | | |
| | | P200462, | | | | | 3.55% | | | |
| | | 96.7% | | | | | gvc MMAE, | | | |
| | | | | | | | RRT | | | |
| | | | | | | | 0.93:3.88% | | | |
| 6 | BC9-LHE- | 0.708 g, | 11 | eq | 1 + 0.1 + 0.1 + 0.1 | eq | 68 h: 0.16% | | | |
| | 0003 | Lot P, | | | | | BCY8234, | | | |
| | | P200462, | | | | | 4.37% | | | |
| | | 96.7% | | | | | gvc MMAE, | | | |
| | | | | | | | RRT | | | |
| | | | | | | | 0.93:1.66% | | | |
| 7 | BC9-LHE- | 1.416 g, | 9 eq DIPEA, | | 1 + 0.1 | eq | 25 h: 0.26% | Column: | 59.1% | 94.7% |
| | 0004 | Lot P, | 4 eq TFA | | | | BCY8234, | 10-40% | | |
| | | P200462, | | | | | 1.53% | ACN/H₂O | | |
| | | 96.7% | | | | | gvc MMAE, | 0.1% AcOH | | |
| | | | | | | | RRT | | | |
| | | | | | | | 0.93:0.79% | | | |
| 8 | BC9-LHE- | 1.416 g, | 8 eq DIPEA, | | 1 + 0.1 | eq | 29.5 h: 0.21% | Column: | 68.5% | 95.5% |
| | 0005 | Lot P, | 3 eq TFA, | | | | BCY8234, | 10-38%, 45% | | |
| | | P200462, | 12 eq water | | | | 1.98% | ACN/H₂O | | |
| | | 96.7% | | | | | gvc MMAE, | 0.1% AcOH | | |
| | | | | | | | RRT | | | |
| | | | | | | | 0.93:1.69% | | | |
| 9 | BC9-LHE- | 0.708 g, | 9 eq DIPEA, | | 1.1 | eq | 1 h: 0.55% | | | |
| | 0006 | Lot P, | 4 eq TFA | | | | BCY8234, | | | |
| | | P200462, | | | | | 1.72% | | | |
| | | 96.7% | | | | | gvc MMAE, | | | |
| | | | | | | | RRT | | | |
| | | | | | | | 0.93:1.04% | | | |
| 10 | BC9-LHE- | 0.708 g, | 9 | eq | 1.1 | eq | 1 h: 0.23% | | | |
| | 0007 | Lot C, | | | | | BCY8234, | | | |
| | | 1909118, | | | | | 1.68% | | | |
| | | 97.9% | | | | | gvc MMAE, | | | |
| | | | | | | | RRT | | | |
| | | | | | | | 0.93:0.87% | | | |

| | | | | | | | | | |
|---|---|---|---|---|---|---|---|---|---|
| 11 | BC9-LHE- | 0.708 g, | 9 eq DIPEA, | 1 | eq | 1 h: 3.45% | Column: | 64.7% | 96.9% |
| | 0008 | Lot P, | 4 eq TFA | | | BCY8234, | 10-38% | | |
| | | P200462, | | | | 2.00% | ACN/H₂O | | |
| | | 96.7% | | | | gvc MMAE, | 0.1% AcOH | | |
| | | | | | | RRT | | | |
| | | | | | | 0.93:0.01% | | | |

### Step 2: Identification of Impurities

Impurity at RRT 0.97: In the chromatogram of the crude BT8009, there is a 4.5% impurity at RRT 0.97. This impurity also exists in the IPC chromatogram. This impurity has been identified as impurity BT8009+OH by LC-MS of BT8009.

Impurity at RRT 0.93: In the chromatogram of final BT8009, there is a 1.4% impurity at RRT 0.93. This impurity also exists in the IPC chromatogram when using excessive gvcMMAE/TBTU. This impurity has been identified as impurity BT8009−H₂O by LC-MS of BT8009. Both Lot C and Lot P BCY8234 were analyzed by LC-MS and confirmed to contain this impurity BCY8234−H₂O. It partially coelutes with the main peak. The Lot C BCY8234 seems to contain more of this impurity.

### Conclusion

A process was developed to produce BT8009 in 44% yield over two steps and 96.9% LC purity. The step 1 process was simplified, and the yield was improved. To minimize the RRT 0.93 impurity, one equivalent of gvcMMAE/TBTU was used for the step 2 reaction. The step 2 filtration and column purification were optimized.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method of preparing a compound of formula I, or a salt thereof, comprising steps of 1) providing fragment F-2 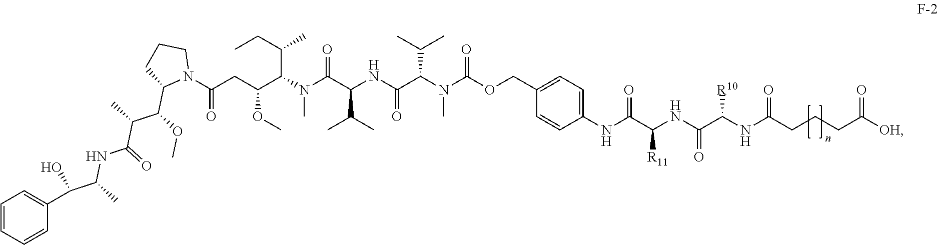
or a salt thereof;
2) reacting fragment F-2 with fragment F-3 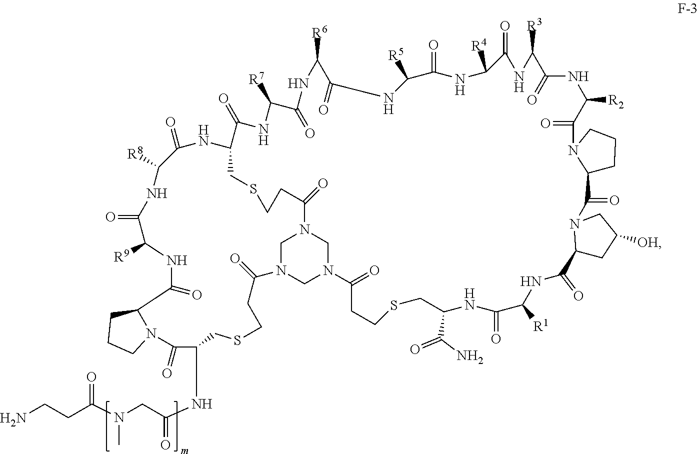
or a salt thereof, to form a compound of formula I 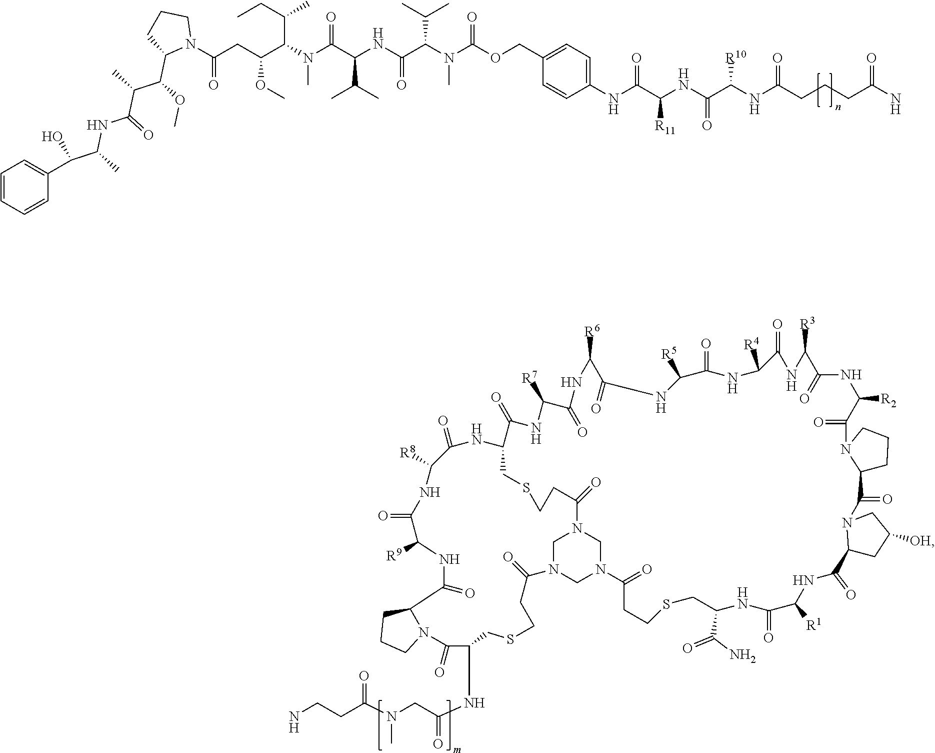
or a salt thereof; and
3) separating the compound of formula I, or a salt thereof, from reaction mixture by precipitation in a non-polar solvent,
wherein:
each of R¹, R², R³, R⁴, R⁵, R⁶, R⁷, R⁸, R⁹, R¹⁰, and R¹¹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
m is 0, 1, 2, 3, 4, 5, 6, 7, 8, 9, 10, 11, 12, 13, 14, or 15; and
n is 0, 1, or 2.

2. The method of claim 1, wherein the step 1) reaction uses about 1 equivalent of fragment F-2.

3. The method of claim 1 or 2, wherein step 2) reaction is in a dipolar aprotic solvent.

4. The method of claim 3, wherein the dipolar aprotic solvent is N,N-dimethylacetamide (DMA).

5. The method of any one of claims 1-4, wherein the non-polar solvent of step 3) is an ether.

6. The method of claim 5, wherein the ether is methyl tert-butyl ether (MTBE).

7. The method of any one of claims 1-6, further comprising purifying the compound of formula I, or a salt thereof, by column chromatography.

8. The method of any one of claims 1-7, wherein an impurity at RRT 0.93 is formed in less than about 5% relative area to a compound of formula I.

9. The method of claim 8, wherein the impurity is less than about 2.5%.

10. The method of claim 9, wherein the impurity is less than about 1%.

11. The method of claim 10, wherein the impurity is less than about 0.5%.

12. The method of claim 11, wherein the impurity is less than about 0.05%.

13. A method of preparing fragment F-2, or a salt thereof, comprising steps of
1) providing fragment F-1 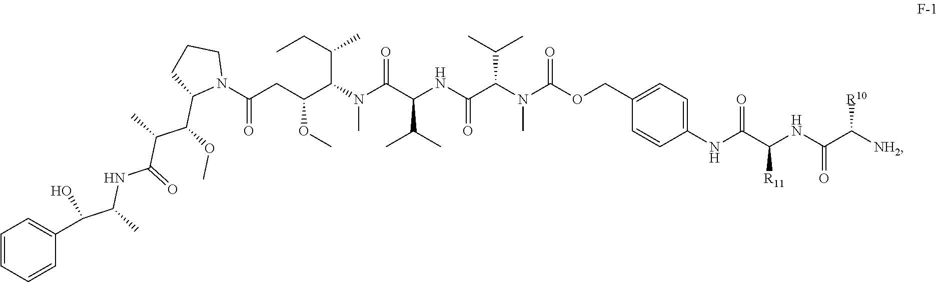
or a salt thereof;
2) reacting fragment F-1 with compound A 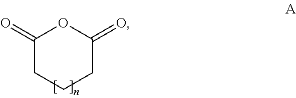
to form fragment F-2 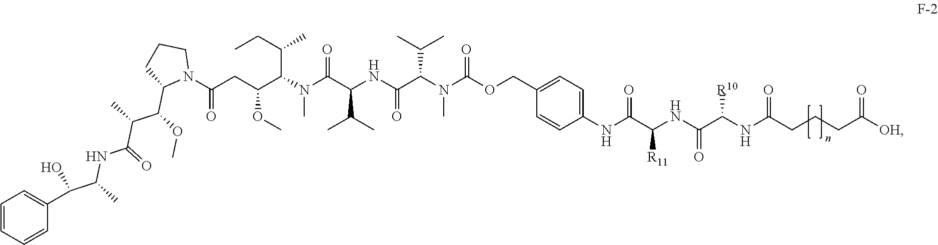
or a salt thereof; and
3) separating fragment F-2, or a salt thereof, from reaction mixture by precipitation in a non-polar solvent,
wherein:
each of R¹⁰ and R¹¹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
n is 0, 1, or 2.

14. The method of claim 13, wherein step 2) reaction is in a dipolar aprotic solvent.

15. The method of claim 14, wherein the dipolar aprotic solvent is N,N-dimethylacetamide (DMA).

16. The method of any one of claims 13-15, wherein the non-polar solvent of step 3) is an ether.

17. The method of claim 16, wherein the ether is methyl tert-butyl ether (MTBE).

18. The method of any one of claims 13-17, further comprising purifying fragment F-2, or a salt thereof, by charging the reaction solution into an acidic saturated brine solution.

19. The method of any one of claim 18, further comprising purifying fragment F-2, or a salt thereof, by column chromatography.

20. The method of any one of claims 1-12, wherein each of R¹, R², R³, R⁴, R⁵, R⁶, R⁷, R⁸, R⁹, R¹⁰, and R¹¹ is independently an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

21. The method of claim 20, wherein R¹ is

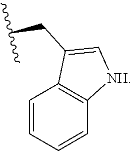

22. The method of claim 20, wherein R² is

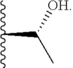

23. The method of claim 20, wherein R³ is

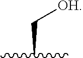

24. The method of claim 20, wherein R⁴ is

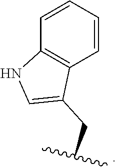

25. The method of claim 20, wherein R⁵ is

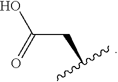

26. The method of claim 20, wherein R⁶ is

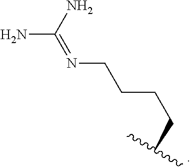

27. The method of claim 20, wherein R⁷ is

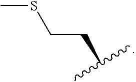

28. The method of claim 20, wherein R⁸ is

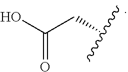

29. The method of claim 20, wherein R⁹ is

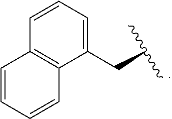

30. The method of claim 20, wherein R¹⁰ is

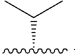

31. The method of claim 20, wherein R¹¹ is

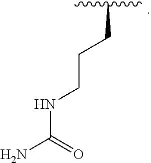

32. The method of any one of claims 20-31, wherein the compound of formula I is

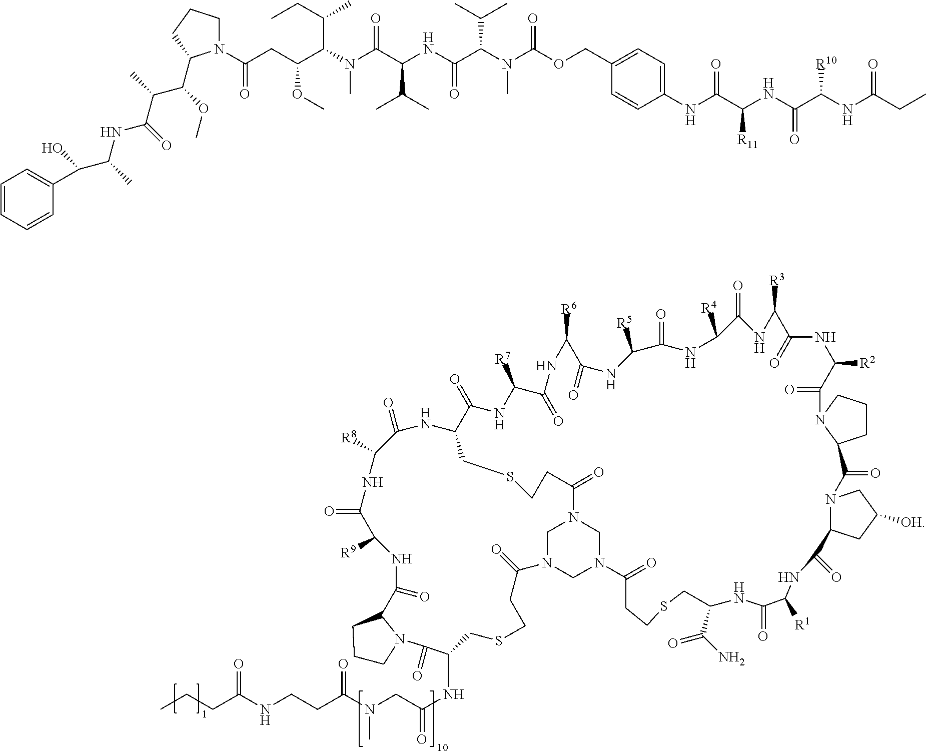

33. The method of any one of claims 1-12, wherein fragment F-3 is 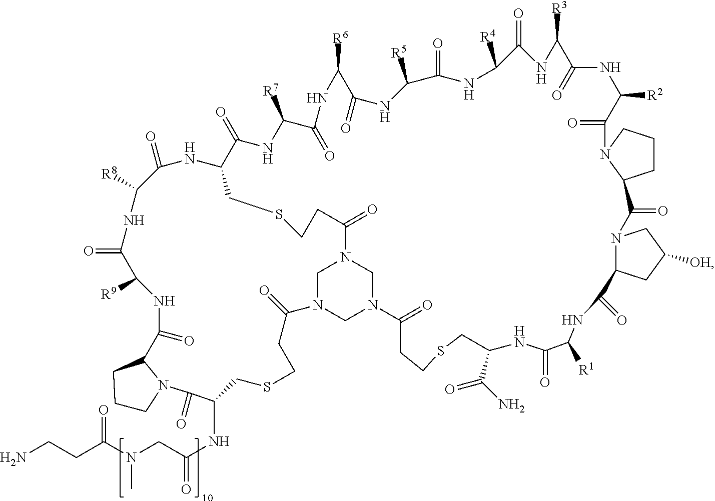
or a salt thereof.

34. The method of any one of claims 1-12, wherein fragment F-2 is 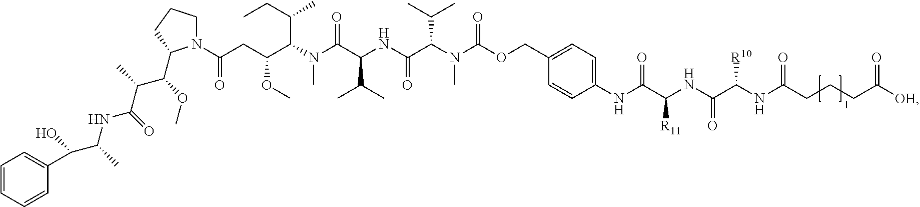
or a salt thereof.

35. The method of claim 33, wherein fragment F-3 is 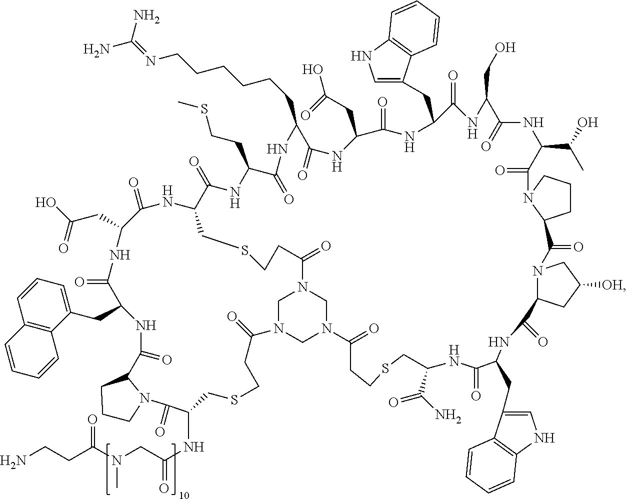
or a salt thereof.

36. The method of claim 34, wherein fragment F-2 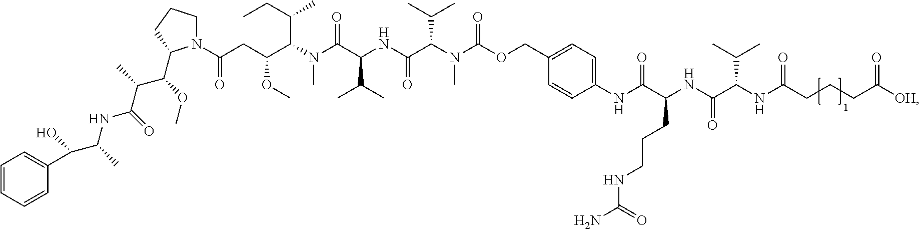
or a salt thereof.

37. The method of any one of claims 1-12 or 20-36, wherein the compound of formula I is BT8009, or a salt thereof.
